SEQUENCE LISTING (78 sequences)

SEQ ID NO: 1 - RNA, 25 nt, Artificial Sequence
  sense_HRRT_non_modi
ccagacuuuguuggauuugaagugc

SEQ ID NO: 2 - RNA, 27 nt, Artificial Sequence
  antisense_HRRT_non_modi
gcacuucaaauccaacaaagucuggca

SEQ ID NO: 3 - RNA, 27 nt, Artificial Sequence
  antisense_HRRT_18_Ome
  modified_base (18): 2′-Ome
gcacuucaaauccaacaaagucuggca

SEQ ID NO: 4 - RNA, 27 nt, Artificial Sequence
  antisense_HRRT_19_Ome
  modified_base (19): 2′-Ome
gcacuucaaauccaacaaagucuggca

SEQ ID NO: 5 - RNA, 27 nt, Artificial Sequence
  antisense_HRRT_18_19_Ome
  modified_base (18)..(19): 2′-Ome
gcacuucaaauccaacaaagucuggca

SEQ ID NO: 6 - RNA, 25 nt, Artificial Sequence
  sense_GFP_non_modi
gcaagcugacccugaaguuaucacc

SEQ ID NO: 7 - RNA, 27 nt, Artificial Sequence
  antisense_GFP_non_modi
ggugauaacuucagggucagcuugcca

SEQ ID NO: 8 - RNA, 27 nt, Artificial Sequence
  antisense_GFP_18_Ome
  modified_base (18): 2′-Ome
ggugauaacuucagggucagcuugcca

SEQ ID NO: 9 - RNA, 27 nt, Artificial Sequence
  antisense_GFP_19_Ome
  modified_base (19): 2′-Ome
ggugauaacuucagggucagcuugcca

SEQ ID NO: 10 - DNA, 21 bp, Artificial Sequence
  HRRT_Forward_primer
gactttgctttccttggtcag

SEQ ID NO: 11 - DNA, 24 bp, Artificial Sequence
  HRRT_Reverse_primer
ggcttatatccaacacttcgtggg

SEQ ID NO: 12 - DNA, 18 bp, Artificial Sequence
  GAPDH_Forward_primer
ggatttggtcgtattggg

SEQ ID NO: 13 - DNA, 19 bp, Artificial Sequence
  GAPDH_Reverse_primer
ggaagatggtgatgggatt

SEQ ID NO: 14 - RNA, 25 nt, Artificial Sequence
  sense_HRRT_19-21bp_modi
  modified_base (21)..(23): 2′-Ome
ccagacuuuguuggauuugaagugc

SEQ ID NO: 15 - RNA, 27 nt, Artificial Sequence
  antisense_HRRT_19-21bp_modi
  modified_base (5)..(7): 2′-Ome
gcacuucaaauccaacaaagucuggca

SEQ ID NO: 16 - RNA, 25 nt, Artificial Sequence
  sense_HRRT_˜15bp_modi
  modified_base (1): 2′-Ome
  modified_base (2): 2′-Ome
  modified_base (6)..(9): 2′-Ome
  modified_base (11)..(12): 2′-Ome
ccagacuuuguuggauuugaagugc

SEQ ID NO: 17 - RNA, 27 nt, Artificial Sequence
  antisense_HRRT_˜15bp_modi
  modified_base (13): 2′-Ome
  modified_base (16): 2′-Ome
  modified_base (26): 2′-Ome
gcacuucaaauccaacaaagucuggca

SEQ ID NO: 18 - RNA, 25 nt, Artificial Sequence
  sense_HRRT_˜15bp+19-21bp_modi
  modified_base (1): 2′-Ome
  modified_base (2): 2′-Ome
  modified_base (6)..(9): 2′-Ome
  modified_base (11)..(12): 2′-Ome
  modified_base (16)..(18): 2′-Ome
ccagacuuuguuggauuugaagugc

SEQ ID NO: 19 - RNA, 27 nt, Artificial Sequence
  antisense_HRRT_˜15bp+19-21bp_modi
  modified_base (7): 2′-Ome
  modified_base (13): 2′-Ome
  modified_base (16): 2′-Ome
  modified_base (26): 2′-Ome
gcacuucaaauccaacaaagucuggca

SEQ ID NO: 20 - RNA, 25 nt, Artificial Sequence
  sense_HRRT_C/U_modi
  modified_base (1)..(2): 2′-Ome
  modified_base (6)..(9): 2′-Ome
  modified_base (11)..(12): 2′-Ome
ccagacuuuguuggauuugaagugc

SEQ ID NO: 21 - RNA, 27 nt, Artificial Sequence
  antisense_HRRT_C/U_modi
  modified_base (11)..(13): 2′-Ome
  modified_base (16): 2′-Ome
  modified_base (21)..(23): 2′-Ome
  modified_base (26): 2′-Ome
gcacuucaaauccaacaaagucuggca

SEQ ID NO: 22 - RNA, 25 nt, Artificial Sequence
  sense_HRRT_Alt_modi
  modified_base (2): 2′-Ome
  modified_base (4): 2′-Ome
  modified_base (6): 2′-Ome
  modified_base (8): 2′-Ome
  modified_base (10): 2′-Ome
  modified_base (12): 2′-Ome
  modified_base (14): 2′-Ome
ccagacuuuguuggauuugaagugc

SEQ ID NO: 23 - RNA, 27 nt, Artificial Sequence
  antisense_HRRT_Alt_modi
  modified_base (11): 2′-Ome
  modified_base (13): 2′-Ome
  modified_base (15): 2′-Ome
  modified_base (17): 2′-Ome
  modified_base (19): 2′-Ome
  modified_base (21): 2′-Ome
  modified_base (23): 2′-Ome
  modified_base (25): 2′-Ome
gcacuucaaauccaacaaagucuggca

SEQ ID NO: 24 - RNA, 25 nt, Artificial Sequence
  sense_GFP_P(+1)P_9_Ome
  modified_base (1): 2′-Ome
  modified_base (4): 2′-Ome
  modified_base (5): 2′-Ome
  modified_base (7): 2′-Ome
  modified_base (9): 2′-Ome
  modified_base (14): 2′-Ome
gcaagcugacccugaaguuaucacc

SEQ ID NO: 25 - RNA, 27 nt, Artificial Sequence
  antisense_GFP_P(+1)P
  modified_base (13)..(16): 2′-Ome
  modified_base (18): 2′-Ome
  modified_base (20): 2′-Ome
  modified_base (23)..(24): 2′-Ome
ggugauaacuucagggucagcuugcca

SEQ ID NO: 26 - RNA, 25 nt, Artificial Sequence
  sense_GFP_P(+1)P
  modified_base (1): 2′-Ome
  modified_base (4): 2′-Ome
  modified_base (5): 2′-Ome
  modified_base (7): 2′-Ome
  modified_base (14): 2′-Ome
gcaagcugacccugaaguuaucacc

SEQ ID NO: 27 - RNA, 25 nt, Artificial Sequence
  sense_HRRT_P(+1)P
  modified_base (1): 2′-Ome
  modified_base (4): 2′-Ome
  modified_base (5): 2′-Ome
  modified_base (7): 2′-Ome
  modified_base (14): 2′-Ome
ccagacuuuguuggauuugaagugc

SEQ ID NO: 28 - RNA, 27 nt, Artificial Sequence
  antisense_HRRT_P(+1)P
  modified_base (13)..(16): 2′-Ome
  modified_base (18): 2′-Ome
  modified_base (20): 2′-Ome
  modified_base (23)..(24): 2′-Ome
gcacuucaaauccaacaaagucuggca

SEQ ID NO: 29 - RNA, 21 nt, Artificial Sequence
  sense_HRRT_siRNA
ccagacuuuguuggauuugtt

SEQ ID NO: 30 - RNA, 21 nt, Artificial Sequence
  antisense_HRRT_siRNA
caaauccaacaaagucuggtt

SEQ ID NO: 31 - RNA, 52 nt, Artificial Sequence
  strand1_Y-RNA_HRRT/FVII_P(+1)P
  modified_base (1): 2′-Ome
  modified_base (4): 2′-Ome
  modified_base (5): 2′-Ome
  modified_base (7): 2′-Ome
  modified_base (14): 2′-Ome
  modified_base (38)..(41): 2′-Ome
  modified_base (43): 2′-Ome
  modified_base (45): 2′-Ome
  modified_base (48)..(49): 2′-Ome
ccagacuuuguuggauuugaagugcggugauguaagacuugagaugauccca

SEQ ID NO: 32 - RNA, 52 nt, Artificial Sequence
  strand2_Y-RNA_FVII/mTTR_P(+1)P
  modified_base (1): 2′-Ome
  modified_base (4)..(5): 2′-Ome
  modified_base (7): 2′-Ome
  modified_base (14): 2′-Ome
  modified_base (38)..(41): 2′-Ome
  modified_base (43): 2′-Ome
  modified_base (45): 2′-Ome
  modified_base (48)..(49): 2′-Ome
ggaucaucucaagucuuacaucacccgucuauuauagagcaagaacacugca

SEQ ID NO: 33 - RNA, 52 nt, Artificial Sequence
  strand3_Y-RNA_mTTR/HRRT_P(+1)P
  modified_base (1): 2′-Ome
  modified_base (4)..(5): 2′-Ome
  modified_base (7): 2′-Ome
  modified_base (14): 2′-Ome
  modified_base (38)..(41): 2′-Ome
  modified_base (43): 2′-Ome
  modified_base (45): 2′-Ome
  modified_base (48)..(49): 2′-Ome
caguguucuugcucuauaauagacggcacuucaaauccaacaaagucuggca

SEQ ID NO: 34 - RNA, 25 nt, Artificial Sequence
  sense_mTTR_P(+1)P
  modified_base (1): 2′-Ome
  modified_base (4)..(5): 2′-Ome
  modified_base (7): 2′-Ome
  modified_base (14): 2′-Ome
caguguucuugcucuauaauagacg

SEQ ID NO: 35 - RNA, 21 nt, Artificial Sequence
  sense_mTTR_siRNA
caguguucuugcucuauaatt

SEQ ID NO: 36 - RNA, 21 nt, Artificial Sequence
  antisense_mTTR_siRNA
uuauagagcaagaacacugtt

SEQ ID NO: 37 - RNA, 21 nt, Artificial Sequence
  sense_FVII_siRNA
ggaucaucucaagucuuactt

SEQ ID NO: 38 - RNA, 21 nt, Artificial Sequence
  antisense_FVII_siRNA
guaagacuugagaugaucctt

SEQ ID NO: 39 - RNA, 25 nt, Artificial Sequence
  sense_mTTR_non_modi
caguguucuugcucuauaauagacg

SEQ ID NO: 40 - RNA, 27 nt, Artificial Sequence
  antisense_mTTR_non_modi
cgucuauuauagagcaagaacacugca

SEQ ID NO: 41 - RNA, 25 nt, Artificial Sequence
  sense_FVII_non_modi
ggaucaucucaagucuuacaucacc

SEQ ID NO: 42 - RNA, 27 nt, Artificial Sequence
  antisense_FVII_non_modi
ggugauguaagacuugagaugauccca

SEQ ID NO: 43 - RNA, 27 nt, Artificial Sequence
  antisense_mTTR_P(+1)P
  modified_base (13)..(16): 2′-Ome
  modified_base (18): 2′-Ome
  modified_base (20): 2′-Ome
  modified_base (23)..(24): 2′-Ome
cgucuauuauagagcaagaacacugca

SEQ ID NO: 44 - RNA, 25 nt, Artificial Sequence
  sense_FVII_P(+1)P
  modified_base (1): 2′-Ome
  modified_base (4)..(5): 2′-Ome
  modified_base (7): 2′-Ome
  modified_base (14): 2′-Ome
ggaucaucucaagucuuacaucacc

SEQ ID NO: 45 - RNA, 27 nt, Artificial Sequence
  antisense_FVII_P(+1)P
  modified_base (13)..(16): 2′-Ome
  modified_base (18): 2′-Ome
  modified_base (20): 2′-Ome
  modified_base (23)..(24): 2′-Ome
ggugauguaagacuugagaugauccca

SEQ ID NO: 46 - RNA, 52 nt, Artificial Sequence; identical to SEQ ID NO: 31
  strand1_Y-RNA_HRRT/FVII_non_modi

SEQ ID NO: 47 - RNA, 52 nt, Artificial Sequence; identical to SEQ ID NO: 32
  strand2_Y-RNA_FVII/mTTR_non_modi

SEQ ID NO: 48 - RNA, 52 nt, Artificial Sequence; identical to SEQ ID NO: 33
  strand3_Y-RNA_mTTR/HRRT_non_modi

SEQ ID NO: 49 - RNA, 25 nt, Artificial Sequence
  sense_TP53_non_modi
accagggcagcuacgguuuaagugc

SEQ ID NO: 50 - RNA, 27 nt, Artificial Sequence
  antisense_TP53_non_modi
gcacuuaaaccguagcugcccugguca

SEQ ID NO: 51 - RNA, 25 nt, Artificial Sequence
  sense_TP53_P(+1)P
  modified_base (1): 2′-Ome
  modified_base (4)..(5): 2′-Ome
  modified_base (7): 2′-Ome
  modified_base (14): 2′-Ome
accagggcagcuacgguuuaagugc

SEQ ID NO: 52 - RNA, 27 nt, Artificial Sequence
  antisense_TP53_P(+1)P
  modified_base (13)..(16): 2′-Ome
  modified_base (18): 2′-Ome
  modified_base (20): 2′-Ome
  modified_base (23)..(24): 2′-Ome
gcacuuaaaccguagcugcccugguca

SEQ ID NO: 53 - DNA, 20 bp, Artificial Sequence
  TP53_Forward_primer
ttccgagagctgaatgaggc

SEQ ID NO: 54 - DNA, 25 bp, Artificial Sequence
  TP53_Reverse_primer
gaagtggagaatgtcagtctgagtc

SEQ ID NO: 55 - RNA, 25 nt, Artificial Sequence
  sense_FVII_C/U_modi
  modified_base (4)..(5): 2′-Ome
  modified_base (7)..(10): 2′-Ome
  modified_base (14)..(15): 2′-Ome
ggaucaucucaagucuuacaucacc

SEQ ID NO: 56 - RNA, 27 nt, Artificial Sequence
  antisense_FVII_C/U_modi
  modified_base (13)..(15): 2′-Ome
  modified_base (20): 2′-Ome
  modified_base (23)..(26): 2′-Ome
ggugauguaagacuugagaugauccca

SEQ ID NO: 57 - RNA, 25 nt, Artificial Sequence
  sense_mTTR_P(+1)P_(-4)
  modified_base (1): 2′-Ome
  modified_base (5): 2′-Ome
  modified_base (7): 2′-Ome
  modified_base (14): 2′-Ome
caguguucuugcucuauaauagacg

SEQ ID NO: 58 - RNA, 25 nt, Artificial Sequence
  sense_mTTR_P(+1)P_(-5)
  modified_base (1): 2′-Ome
  modified_base (4): 2′-Ome
  modified_base (7): 2′-Ome
  modified_base (14): 2′-Ome
caguguucuugcucuauaauagacg

SEQ ID NO: 59 - RNA, 25 nt, Artificial Sequence
  sense_mTTR_P(+1)P_(-7)
  modified_base (1): 2′-Ome
  modified_base (4)..(5): 2′-Ome
  modified_base (14): 2′-Ome
caguguucuugcucuauaauagacg

SEQ ID NO: 60 - RNA, 25 nt, Artificial Sequence
  sense_mTTR_P(+1)P_(-14)
  modified_base (1): 2′-Ome
  modified_base (4)..(5): 2′-Ome
  modified_base (7): 2′-Ome
caguguucuugcucuauaauagacg

SEQ ID NO: 61 - RNA, 25 nt, Artificial Sequence
  sense_HRRT_P(-1)P
  modified_base (4)..(5): 2′-Ome
  modified_base (7): 2′-Ome
  modified_base (14): 2′-Ome
ccagacuuuguuggauuugaagugc

SEQ ID NO: 62 - RNA, 27 nt, Artificial Sequence
  antisense_HRRT_P(-1)P
  modified_base (13)..(16): 2′-Ome
  modified_base (18): 2′-Ome
  modified_base (20): 2′-Ome
  modified_base (23)..(24): 2′-Ome
gcacuucaaauccaacaaagucuggca

SEQ ID NO: 63 - RNA, 25 nt, Artificial Sequence
  sense_GFP_P(-1)P
  modified_base (4)..(5): 2′-Ome
  modified_base (7): 2′-Ome
  modified_base (14): 2′-Ome
gcaagcugacccugaaguuaucacc

SEQ ID NO: 64 - RNA, 27 nt, Artificial Sequence
  antisense_GFP_P(-1)P
  modified_base (13)..(16): 2′-Ome
  modified_base (18): 2′-Ome
  modified_base (20): 2′-Ome
  modified_base (23)..(24): 2′-Ome
ggugauaacuucagggucagcuugcca

SEQ ID NO: 65 - RNA, 25 nt, Artificial Sequence
  sense_FVII_P(-1)P
  modified_base (4)..(5): 2′-Ome
  modified_base (7): 2′-Ome
  modified_base (14): 2′-Ome
ggaucaucucaagucuuacaucacc

SEQ ID NO: 66 - RNA, 27 nt, Artificial Sequence
  antisense_FVII_P(-1)P
  modified_base (13)..(16): 2′-Ome
  modified_base (18): 2′-Ome
  modified_base (20): 2′-Ome
  modified_base (23)..(24): 2′-Ome
ggugauguaagacuugagaugauccca

SEQ ID NO: 67 - RNA, 25 nt, Artificial Sequence
  sense_mTTR_P(-1)P
  modified_base (4)..(5): 2′-Ome
  modified_base (7): 2′-Ome
  modified_base (14): 2′-Ome
caguguucuugcucuauaauagacg

SEQ ID NO: 68 - RNA, 27 nt, Artificial Sequence
  antisense_mTTR_P(-1)P
  modified_base (13)..(16): 2′-Ome
  modified_base (18): 2′-Ome
  modified_base (20): 2′-Ome
  modified_base (23)..(24): 2′-Ome
cgucuauuauagagcaagaacacugca

SEQ ID NO: 69 - RNA, 52 nt, Artificial Sequence; identical to SEQ ID NO: 31
  strand1_Y-RNA_HRRT/FVII_P(-1)P
  modified_base (4)..(5): 2′-Ome
  modified_base (7): 2′-Ome
  modified_base (14): 2′-Ome
  modified_base (38)..(41): 2′-Ome
  modified_base (43): 2′-Ome
  modified_base (45): 2′-Ome
  modified_base (48)..(49): 2′-Ome

SEQ ID NO: 70 - RNA, 52 nt, Artificial Sequence; identical to SEQ ID NO: 32
  strand2_Y-RNA_FVII/mTTR_P(-1)P
  modified_base (4)..(5): 2′-Ome
  modified_base (7): 2′-Ome
  modified_base (14): 2′-Ome
  modified_base (38)..(41): 2′-Ome
  modified_base (43): 2′-Ome
  modified_base (45): 2′-Ome
  modified_base (48)..(49): 2′-Ome

SEQ ID NO: 71 - RNA, 23 nt, Artificial Sequence
  synthetic_sense_GFP_non_modi_23nt
gcaagcugacccugaaguuauca

SEQ ID NO: 72 - RNA, 25 nt, Artificial Sequence
  synthetic_antisense_GFP_non_modi_25nt
ugauaacuucagggucagcuugcca

SEQ ID NO: 73 - RNA, 23 nt, Artificial Sequence
  synthetic_sense_GFP_P(+1)P_23nt
  modified_base (1): 2′-Ome
  modified_base (4): 2′-Ome
  modified_base (5): 2′-Ome
  modified_base (7): 2′-Ome
  modified_base (14): 2′-Ome
gcaagcugacccugaaguuauca

SEQ ID NO: 74 - RNA, 25 nt, Artificial Sequence
  synthetic_antisense_GFP_P(+1)P_25nt
  modified_base (11)..(14): 2′-Ome
  modified_base (16): 2′-Ome
  modified_base (18): 2′-Ome
  modified_base (21)..(22): 2′-Ome
ugauaacuucagggucagcuugcca

SEQ ID NO: 75 - RNA, 30 nt, Artificial Sequence
  synthetic_sense_GFP_non_modi_30nt
gcaagcugacccugaaguuaucaccucacc

SEQ ID NO: 76 - RNA, 32 nt, Artificial Sequence
  synthetic_antisense_GFP_non_modi_32nt
ggugaggugauaacuucagggucagcuugcca

SEQ ID NO: 77 - RNA, 30 nt, Artificial Sequence; identical to SEQ ID NO: 75
  synthetic_sense_GFP_P(+1)P_30nt
  modified_base (1): 2′-Ome
  modified_base (4): 2′-Ome
  modified_base (5): 2′-Ome
  modified_base (7): 2′-Ome
  modified_base (14): 2′-Ome

SEQ ID NO: 78 - RNA, 32 nt, Artificial Sequence; identical to SEQ ID NO: 76
  synthetic_antisense_GFP_P(+1)P_32nt
  modified_base (18)..(21): 2′-Ome
  modified_base (23): 2′-Ome
  modified_base (25): 2′-Ome
  modified_base (28)..(29): 2′-Ome

(19) United States
(12) Patent Application Publication
(10) Pub. No.: US 2023/0392151 A1
(43) Pub. Date: Dec. 7, 2023

(54) NUCLEIC ACID MOLECULES WITH INCREASED GENE SILENCING ACTIVITY AND USES THEREOF

(71) Applicant: Ewha Universty - Industry Collaboration Foundation, Seoul (KR)

(72) Inventors: Hyukjin LEE, Seoul (KR); Bora JANG, Seoul (KR); Hyunsook KIM, Seoul (KR); Hye-jin JANG, Changwon-si (KR)

(21) Appl. No.: 18/266,463

(22) PCT Filed: Dec. 17, 2021
(86) PCT No.: PCT/KR2021/019345

§ 371 Date: Jun. 9, 2023

(30) Foreign Application Priority Data
Dec. 18, 2020 (KR) 10-2020-0179036

(51) Int. Cl. C12N 15/113

(52) U.S. Cl. CPC C12N 15/113; C12N 2310/321; C12N 2310/14; C12N 2310/344; C12N 2320/30

(57) ABSTRACT

The present invention relates to nucleic add molecules with increased gene silencing activity and uses thereof, wherein a double-stranded nucleic acid molecule or radial nucleic acid molecule comprising a chemically modified nudeotide at a specific position may have effectively increased gene silencing activity and in vivo stability for a target gene.

### CROSS-REFERENCE TO RELATED APPLICATION(S)

This patent application claims priority to and the benefit of Korean Patent Application No. 10-2020-0179036 filed with the Korean Intellectual Property Office on Dec. 18, 2020, the disdosure of which is incorporated herein by reference in its entirety.

### TECHNICAL FIELD

The present application relates to a nucleic add molecule with increased gene silendng activity and a use thereof.

### BACKGROUND ART

RNA interference refers to the process of sequence-specific post-transcriptional gene silendng mediated by short interfering RNAs (siRNAs) in animals, wherein a double-stranded RNA composed of a sense strand having a sequence homologous to the mRNA of a target gene (gene of interest) and an antisense strand having a sequence complementary thereto is introduced into a cell or the like to induce degradation of mRNA of the target gene, whereby the expression of the target gene is downregulated.

Double-stranded RNA (dsRNA) agents possessing strand lengths of 25 to 35 nucleotides have been described as effective inhibitors of target gene expression in mammalian cells. When introduced into cells, dsRNA agents of such length are processed by the ribonuclease Dicer into small RNAs 21-23 bp long. The truncated form of small RNA is called siRNA (short interfering RNA). The siRNA cleaved in the cytoplasm is incorporated into the RNA-induced silendng complex (RISC) while the sense strand of siRNA is degraded by active Argonaute-2. The antisense strand-associated, active RISC complex binds complementarily to and degrades the target mRNA, resulting in interference with protein formation.

The RNAi field is evaluated to have much greater potential than ribozymes because of its ability to downregulate the expression of substantially all genes. RNAi therapeutics can be used as a treatment for diseases that were difficult to treat with existing drugs without restrictions, emerging as a new solution to incurable diseases and being recognized as a next-generation future drug technology.

However, despite these expectations, development of RNAi therapeutics is limited due to various problems. The problems, including instability of siRNA in vivo (e.g., degradation by intracellular nuclease), inefficiency of delivery, and non-specific action (e.g., too long dsRNA binds in non-specifical manners to induce an interferon response), significantly inhibits the therapeutic effect of RNAi therapeutics. In order to overcome these limitations of RNAi, research into increasing RNA stability is ongoing. Therefore, there is a need to develop dsRNA with increased gene silencing activity while overcoming the biological barriers of RNAi therapeutics.

### RELATED ART DOCUMENT

### Patent Literature

(Patent literature 1) Korean Patent No. 10-2007-0028363 A

### DISCLOSURE

### Technical Problem

Leading to the present disclosure, intensive and thorough research conducted by the present inventors found that a double-stranded nudeic acid molecule having a chemically modified nucleotide at a specific position or a radial nucleic acid molecule having the double-stranded nucleic acid molecule incorporated into K arm moieties (e.g., K is an integer, 21.4) which radially extend exhibited remarkably improved gene silencing activity for a target gene in vitro and/or in vivo.

An aspect of the present application is to provide a double-stranded nucleic acid molecule including a sense strand having a nucleotide chemically modified at a specific position and an antisense strand having a sequence complementary to the sense strand.

Another aspect of the present application is to provide a radial nudeic acid molecule induding the double-stranded nucleic acid molecule.

Another aspect of the present application is to provide a use of the double-stranded nudeic acid molecule and/or the radial nucleic acid molecule for the inhibition of gene expression.

Another aspect of the present application is to provide a composition including the double-stranded nucleic add molecule and/or the radial nucleic acid molecule for the inhibition of gene expression.

Another aspect of the present application is to provide a use of the double-stranded nucleic add molecule and/or the radial nucleic acid molecule in preparing a composition for the inhibition of gene expression.

Another aspect of the present application is to provide a method for inhibiting gene expression, the method including a step of administering the double-stranded nucleic acid molecule and/or the radial nucleic acid molecule to a subject in need of inhibiting gene expression.

Another aspect of the present application is to provide a method for the preparation of the double-stranded nucleic acid molecule and/or the radial nudeic add molecule.

### Technical Solution

The term “RNAi” or “RNA interference” refers to a biological process mediated by a short interfering nucleic acid molecule to inhibit or downregulate the expression of a gene in cells, as generally known in the art. For example, the term may mean the mechanism in which double-stranded RNA (dsRNA) composed of a strand having a sequence homologous to the mRNA of a target gene and a strand having a sequence complementary thereto is introduced into cells to induce the degradation of the target gene mRNA, with the consequent downregulation of expression of the target gene. As used herein, the term “small interfering RNA” (siRNA) refers to a short double-stranded RNA (dsRNA) that mediates sequence-specifically efficient gene silencing.

As used herein, the term “nudeic acid” refers to deoxyribonucleotides, ribonucleotides, or modified nucleotides, and polymers thereof in single- or double-stranded form and is intended to encompass nucleic acids containing known nucleotide analogs or modified backbone residues or linkages. Examples of nucleic adds or polynucleotides may include single-, double-, or multi-stranded DNA or RNA, genomic DNA, cDNA, DNA-RNA hybrids, and polymers with purine and pyrimidine bases or other natural, chemically or biochemically modified, non-natural or derivatized nucleotide bases.

As used herein, the term “nucleotide” is used as recognized in the art to include those with natural bases (standard), and modified bases well known in the art. Such bases are generally located at the 1′ position of a nucleotide sugar moiety. Nucleotides generally comprise a base, sugar, and a phosphate group. The nudeotides can be unmodified or modified at the sugar, phosphate, and/or base moiety (also referred to interchangeably as nucleotide analogs, modified nucleotides, non-natural nucleotides, non-standard nudeotides and the like).

By “hybridizable”, “complementary”, or “substantially complementary”, it is meant that a nucleic acid (e.g., RNA, DNA) comprises a sequence of nudeotides that can non-covalently bind to another nucleic acid in a sequence-specific, antiparallel manner (i.e., a nucleic acid binds specifically to a complementary nucleic add), that is, can form Watson-Crick base pairs and/or G/U base pairs, “anneal”, or “hybridize” with another nucleic add, under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule: guanine (G) can also base pair with uracil (U). For example, G/U base-pairing is partially responsible for the degeneracy (i.e., redundancy) of the genetic code in the context of tRNA anti-codon base-pairing with codons in mRNA.

The term “antisense strand”, as used herein, refers to a polynucleotide that is substantially or 100% complementary to a target gene. For example, an antisense strand may be complementary, partially or in its entirety, to a molecule of messenger RNA (mRNA), an RNA sequence other than mRNA (e.g., microRNA, piwiRNA, tRNA, rRNA, and hnRNA, siRNA, miRNA, shRNA, DsiRNA, IsiRNA, ss-siRNA, piRNA, endo-siRNA, or asiRNA), or a sequence of DNA that is either coding or non-coding. The terms “antisense strand” and “guide strand” are used interchangeably herein. The guide strand is a single-stranded portion sequenced for the purpose of inhibiting a target and is associated with an Argonaute protein, serving to guide the Argonaute complex to recognize a target gene.

As used herein, the term “sense strand” refers to a polynucleotide that has the same nudeotide sequence, partially or in its entirety, as a target nucleotide sequence. For example, a sense strand has the same nudeotide sequence, partially or in its entirety, as a molecule of messenger RNA (mRNA), an RNA sequence other than mRNA (e.g., microRNA, piwiRNA, tRNA, rRNA, and hnRNA, siRNA, miRNA, shRNA, DsiRNA, IsiRNA, ss-siRNA, piRNA, endo-siRNA, or asiRNA), or a sequence of DNA that is either coding or non-coding. The “sense strand” and “passenger strand” may be used interchangeably. The passenger strand forms a double-stranded structure with the guide strand among the nucleic acid molecules according to an embodiment, and serves as a passenger to help the guide strand bind to the Argonaute protein.

As used herein, the terms “Dicer substrate nucleic add” and “Dicer substrate RNA” (ribonucleic acid) refer to a nucleic acid that is considered to be recognized and processed by a Dicer enzyme in the RNA interference (RNAi) pathway.

Meant by the term “chemical modification” herein is any modification to the chemical structure of a nucleotide different from that of a native nudeic acid, nucleotide, DNA, and/or RNA.

As used herein, a nucleotide at the nth position (or at position n) from the 5′ end of a sense strand (region), antisense strand (region), or polynucleotide strand means a nucleotide that is counted nth from the 5′ end of the sense strand, antisense strand, or polynucleotide strand.

Below, a detailed description will be given of the present disdosure.

An aspect may provide:
a double-stranded nucleic acid induding a sense strand and an antisense strand complementary to the sense strand (e.g., complementary to the entirety ora part of the sense strand) wherein a nucleotide at a specific position in the sense strand and/or the antisense to strand is chemically modified; or
a radial nucleic acid molecule including a plurality (e.g., 2 to 5) of the double-stranded nudeic acid molecule (e.g., a radial nudeic acid molecule having K arms extending radially, each arm possessing the nudeic acid molecule).

In an embodiment, the sense strand may a single-stranded polynucleotide with a is length of 19 to 70 nt (nudeotides), 20 to 70 nt, 21 to 70 nt, 22 to 70 nt, 23 to 70 nt, 25 to 70 nt, 19 to 66 nt, 20 to 66 nt, 21 to 66 nt, 22 to 66 nt, 23 to 66 nt, 25 to 66 nt, 19 to 60 nt, 20 to 60 nt, 21 to 60 nt, 22 to 60 nt, 23 to 60 nt, 25 to 60 nt, 19 to 55 nt, 20 to 55 nt, 21 to 55 nt, 22 to 55 nt, 23 to 55 nt, 25 to 55 nt, 19 to 52 nt, 20 to 52 nt, 21 to 52 nt, 22 to 52 nt, 23 to 52 nt, 25 to 52 nt, 19 to 50 nt, 20 to 50 nt, 21 to 50 nt, 22 to 50 nt, 23 to 50 nt, 25 to 50 nt, 19 to 45 20 nt, 20 to 45 nt, 21 to 45 nt, 22 to 45 nt, 23 to 45 nt, 25 to 45 nt, 19 to 40 nt, 20 to 40 nt, 21 to 40 nt, 22 to 40 nt, 23 to 40 nt, 25 to 40 nt, 19 to 38 nt, 20 to 38 nt, 21 to 38 nt, 22 to 38 nt, 23 to 38 nt, 25 to 38 nt, 19 to 36 nt, 20 to 36 nt, 21 to 36 nt, 22 to 36 nt, 23 to 36 nt, 25 to 36 nt, 19 to 35 nt, 20 to 35 nt, 21 to 35 nt, 22 to 35 nt, 23 to 35 nt, 25 to 35 nt, 19 to 30 nt, 20 to 30 nt, 21 to 30 nt, 22 to 30 nt, 23 to 30 nt, 25 to 30 nt, 19 to 28 nt, 20 to 28 nt, 21 to 28 nt, 22 to 25 28 nt, 23 to 28 nt, 25 to 28 nt, 19 to 25 nt, 20 to 25 nt, 21 to 25 nt, 22 to 25 nt, 23 to 25 nt, or 25 nt.

In an embodiment, the antisense strand may be a single-stranded polynucleotide with a length of 20 to 70 nt, 21 to 70 nt, 22 to 70 nt, 23 to 70 nt, 25 to 70 nt, 27 to 70 nt, 20 to 66 nt, 21 to 66 nt, 22 to 66 nt, 23 to 66 nt, 25 to 66 nt, 27 to 66 nt, 20 to 60 nt, 21 to 60 nt, 22 to nt, 23 to 60 nt, 25 to 60 nt, 27 to 60 nt, 20 to 55 nt, 21 to 55 nt, 22 to 55 nt, 23 to 55 nt, 25 to 55 nt, 27 to 55 nt, 20 to 52 nt, 21 to 52 nt, 22 to 52 nt, 23 to 52 nt, 25 to 52 nt, 27 to 52 nt, to 50 nt, 21 to 50 nt, 22 to 50 nt, 23 to 50 nt, 25 to 50 nt, 27 to 50 nt, 20 to 45 nt, 21 to 45 nt, 22 to 45 nt, 23 to 45 nt, 25 to 45 nt, 27 to 45 nt, 20 to 40 nt, 21 to 40 nt, 22 to 40 nt, 23 to nt, 25 to 40 nt, 27 to 40 nt, 20 to 38 nt, 21 to 38 nt, 22 to 38 nt, 23 to 38 nt, 25 to 38 nt, 27 to 38 nt, 20 to 36 nt, 21 to 36 nt, 22 to 36 nt, 23 to 36 nt, 25 to 36 nt, 27 to 36 nt, 20 to 35 nt, 21 to 35 nt, 22 to 35 nt, 23 to 35 nt, 25 to 35 nt, 27 to 35 nt, 20 to 30 nt, 21 to 30 nt, 22 to 30 nt, 23 to 30 nt, 25 to 30 nt, 27 to 30 nt, 20 to 27 nt, 21 to 27 nt, 22 to 27 nt, 23 to 27 nt, 25 to 27 nt, or 27 nt.

In an embodiment, the antisense strand may include a sequence complementary to the sense strand. For instance, the antisense strand may consist of or consist essentially of a nucleic acid sequence 60% or more, 65% or more, 70% or more, 75% or more, 80% or more, 85% or more, 90% or more, 92% or more, 94% or more, 95% or more, 96% or more, 97% or more, 98% or more, 98.5% or more, 99% or more, 99.5% or more, 99.8% or more, 99.9% or more, or 100% complementary to the overall or partial nudeic acid sequence of the sense strand, so that the antisense strand can bind to (hybrid with) the sense strand.

In one embodiment, the “specific position” for the chemically modified nucleotide in the sense strand induded in the double-stranded nudeic acid (or radial nucleic acid molecule) may refer to the following positions:

(1) at least one (e.g., one or more, two or more, three or more, four or more, or all of the five) selected from the group consisting of 1^{st}, 4^{th}, 5^{th}, 7^{th}, and 14^{th} positions from the 5′ end of a sense strand induded in the double-stranded nucleic acid molecule.

In one embodiment, the “specific position” for the chemically modified nucleotide in the antisense strand induded in the double-stranded nucleic add (or radial nucleic acid molecule) may refer to the following positions:
(1) a position of a nudeotide in the antisense strand, which binds complementarily to a nucleotide at least one (e.g., one or more, two or more, four or more, five or more, six or more, seven or more, or all of the eight) consisting of 2^{nd}, 3^{rd}, 6^{th}, 8^{th}, and 10^{th} to 13^{th} positions from the 5′ end of the sense strand; or
(2) at least one (e.g., one or more, two or more, four or more, five or more, six or more, seven or more, or all of the eight) selected from the group consisting of 8^{th} to 11^{th}, 13^{th}, 15^{th}, 18^{th}, and 19^{th} positions from the 5′ end of the antisense strand among the products generated through cleavage of the double-stranded nucleic acid molecule by Dicer.

Herein, the position in the antisense strand complementary to the position counted from the 5′ end of the sense strand may correspond to (or may be used interchangeably used with) the position counted from the 5′ end of the antisense strand among products generated by Dicer deavage (or cleaved products), as shown in Table 1, below. For instance, the position in the antisense strand complementary to the 2^{nd} position from the 5′ end of the sense strand may correspond to or may be used interchangeably with the 19^{th} position from the 5′ end of the antisense strand among the products produced by Dicer cleavage (or cleaved products).

TABLE 1

| Position counted from | |
|---|---|
| the 5′ end of sense | |
| strand complementary | Position counted from |
| to antisense strand in | the 5′ end of antisense |
| double-stranded | strand among cleaved |
| nucleic acid | products |
| 1 | 20 |
| 2 | 19 |
| 3 | 18 |
| 4 | 17 |
| 5 | 16 |
| 6 | 15 |
| 7 | 14 |
| 8 | 13 |
| 9 | 12 |
| 10 | 11 |
| 11 | 10 |
| 12 | 9 |
| 13 | 8 |
| 14 | 7 |
| 15 | 6 |
| 16 | 5 |
| 17 | 4 |
| 18 | 3 |
| 19 | 2 |
| 20 | 1 |

An aspect may provide a double-stranded nucleic acid comprising a sense strand and an antisense strand including a sequence complementary to all or part of the sense strand,
wherein the sense strand includes a chemically modified nucleotide at one or more positions selected from the group consisting of 4^{th} 5^{th}, 7^{th}, and 14^{th} positions from the 5′ end thereof, and
the antisense strand includes a chemically modified nudeotide at a position complementary to a nucleotide present at at least one selected from the group consisting 2^{nd}, 8^{th} and 10^{th} to 13^{th} positions from the 5′ end of the sense strand.

In an embodiment, the sense strand included in the double-stranded nucleic acid molecule may bear a chemically modified nucleotide (e.g., nucleotide with the sugar moiety modified with 2′-O-methyl) ora chemically unmodified nucleotide at the 1^{st} position from the end thereof.

The double-stranded nudeic acid molecule according to an embodiment may indude is a sense strand and an antisense strand possessing the following characteristics:
the sense strand bears chemically modified nucleotides at 1^{st}, 4^{th}, 5^{th}, 7^{th} and 14^{th} positions from the 5′ end thereof, and
the antisense strand bears chemically modified nucleotides at positions corresponding to 2^{nd}, 3^{rd}, 6^{th}, 8^{th} and 10^{th} to 13^{th} positions from the 5′ end in the sense strand (or at 8^{th} to 11^{th}, 13^{th}, 15^{th}, 18^{th} and 19^{th} positions from the 5′ end of the antisense strand among the deaved products) wherein the chemically modified nucleotides bind complementarily to the nucleotides of the sense strand at the corresponding positions.

In an embodiment, the sense strand included in the double-stranded nucleic acid molecule may bear a chemically unmodified nucleotide at one or more positions selected from the group consisting of 2^{nd}, 3^{rd}, 6^{th}, 8^{th} to 13^{th}, and 15^{th} or higher positions (e.g., 15^{th} to 36^{th} or 15^{th} to 25^{th} positions) from the 5′ end of the sense strand.

In an embodiment, the antisense strand induded in the double-stranded nucleic acid molecule may bear a chemically unmodified nucleotide at least one position selected from the group consisting of 1^{st}, 4^{th}, 5^{th}, 7^{th}, 9^{th} and 14^{th} or higher positions (e.g., 14^{th} to 36^{th} or 14^{th} to 25^{th} positions) from the 5′ end in the antisense strand, or
at one or more positions selected from the group consisting of 7^{th} or lower (e.g., 1^{st} to 7^{th}) 12^{th}, 14^{th}, 16^{th}, 17^{th}, and 20^{th} or higher (e.g., 20^{th} to 22^{nd}) positions from the 5′ end in the antisense strand among products generated by Dicer cleavage.

As used herein, the term “chemically unmodified” means having the same configuration as in the nucleotides present in native or naturally occurring nucleic acids.

In an embodiment, a double-stranded nucleic acid induding a sense strand in which the nucleotide at the 9^{th} position from the 5′ end of the sense strand is chemically unmodified may have higher gene silencing activity than a double-stranded nucleic acid induding a sense strand in which the nucleotide at the 9^{th} position from the 5′ end of the sense strand is chemically modified.

In an embodiment, the double-stranded nucleic add molecule (or radial nucleic acid molecule) may exhibit at least one selected from the group consisting of the following features (1) to (8) and, specifically, may maintain, improve, or reduce at least one from the group consisting of the following features (1) to (8), compared to a double-stranded nucleic acid the nucleotides of which are all chemically unmodified or a double-stranded nucleic acid chemically modified by an already known method (for example, alternating modification and/or C/U sequence-based modification):
(1) maintenance and/or increase of interaction between the double-stranded nucleic acid and Dicer in vivo and in vitro (for example, maintenance and/or increase of the rate of deavage of double-stranded nudeic adds by Dicer);
(2) maintenance and/or increase of target gene silencing activity in vitro;
(3) increase in target gene silencing activity in vivo;
(4) decrease in off-target effect;
(5) decrease in degradation by nuclease (e.g., RNase) in vivo;
(6) increase in intracellular uptake;
(7) increase in stability in vitro and/or in vivo (e.g., increase in serum stability); and/or
(8) decrease in immune response (e.g., induction of TLR-mediated immune response within endosomes by the double-stranded nudeic add molecule or induction of PKR-mediated immune response by the double-stranded nucleic acid molecule released into the cytosol).

As used herein, the term “alternating modification” refers to a method of chemically modifying adjacent nucleotides in an alternating manner. By way of example, according to alternating modification, nudeotides located at odd positions from the 5′ end in the sense is strand are chemically modified while nucleotides in the antisense strand, complementary to nucleotides located at even positions from the 5′ end in the sense strand, are chemically modified. The term “sequence-based modification” refers to a method of chemically modifying nucleotides induding C and nucleotides including U.

The term “off-target effect” is intended to encompass the mRNA degradation of unexpected mRNA other than a target or the downregulation of expression of a gene other than a target by a sense strand in the double-stranded nucleic acid, and the degradation of an off-target mRNA to the downregulation of a gene other than a target as an antisense strand in the double-stranded nucleic acid binds to an off-target.

In an embodiment, the double-stranded nucleic add molecule (or radial nucleic acid molecule) may exhibit the same, increased, or decreased effect in terms of at least one selected from the group consisting of the properties (1) and (8), compared to siRNA (dsRNA with gene regulatory activity, not processed by Dicer because of its short length) for the same target gene.

As used herein, the term “chemical modification” in context with a nucleotide (or nudeic add) may mean that a sugar, a base, a linker between nudeotides, or a combination thereof included in the nucleotide (or nucleic acid) is chemically modified. Chemical modification is not particularly limited, and those skilled in the art to which the present invention pertains can synthesize and modify the nucleotide (or nucleic acid) in a desired manner using methods known in the art. Non-limiting examples of modified bases that can be introduced into nucleic acid molecules indude hypoxanthine, purine, pyridin-4-one, pyridin-2-one, phenyl, pseudouradl, 2,4,6-trimethoxy benzene, 3-methyluracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine), or 6-azapyrimidines or 6-alkylpyrimidines (e.g., 6-methyluridine), propyne, and others (Burgin, et al., Biochemistry 1996; Uhlman & Peyman, supra). The term “modified base” means a nucleotide base other than adenine, guanine, thymine, cytosine, and uracil at the 1′ position, or an equivalent thereof.

The chemical modification may be characterized in that the hydroxyl group at the 2′ position of the ribose moiety in at least one nucleotide included in a nucleic acid molecule is substituted with any one of a hydrogen atom, a fluorine atom, an —O-alkyl group, an —O-acyl group, and an amino group. The substitution may be carried out with any one of —Br, —Cl, —R, —R′OR, —SH, —SR, —N3, and —CN (R=alkyl, aryl, alkylene), but with no limitations thereto.

In one embodiment, the chemically modified nucleotide may have a sugar moiety modified with at least one selected from the group consisting of 2′-O-methyl, 2′-methoxyethoxy, 2′-fluoro, 2′-allyl, 2′-O-[2-(methyl amino)-2-oxoethyl], 4′-thio, 4′-CH2-O-2′-bridge, 4′-(CH2)2-O-2′-bridge, 2′-LNA, 2′-amino, and 2′-O-(N-methylcarbamate).

A double-stranded nucleic acid molecule according to an embodiment (or a radial nucleic acid molecule according to an embodiment) may be endogenously cleaved by Dicer. A double-stranded nucleic acid molecule according to an embodiment (or a radial nucleic acid molecule according to an embodiment) may be a substrate of Dicer. As used herein, “Dicer” refers to an endonuclease that is a part of the RNase III family and deaves double-stranded RNA (dsRNA) or precursor microRNA (pre-miRNA) into short double-stranded RNA fragments 19-25 base pairs long (i.e., miRNA or siRNA with gene silencing activity).

Each sense strand and/or antisense strand included in a double-stranded nucleic acid molecule according to an embodiment (or a double-stranded nudeic acid molecule included in the radial nucleic acid molecule according to one embodiment) has a predetermined length (e.g., 19 nt, 20 nt, 21 nt, 22 nt, 23 nt, 24 nt, or 25 nt) or longer. When administered to a subject, the double-stranded nudeic add molecule is recognized as “long double-stranded RNA” (dsRNA) and deaved by Dicer to the processed length of about 19 to 25 nt. (step \textcircled{1}, Dicer processing).

As used herein, “subject”, “individual”, or “patient” may refer to an organism to which the nucleic acid molecule (double-stranded nucleic add molecule and/or radial nucleic acid molecule) according to an embodiment can be administered. The subject may be a mammalian (e.g., human) or a mammalian cell (e.g., a human cell), an organism that is a donor or recipient of an explant cell, or the cell itself.

As used herein, the term “Dicer cleavage site” refers to a site at which Dicer cleaves a double-stranded nucleic acid molecule (or radial nucleic acid molecule) according to an embodiment. In one embodiment, Dicer contains two RNase III domains, which are typically capable of cleaving both the sense and antisense strands of a double-stranded nucleic acid molecule (e.g., double-stranded RNA).

In an embodiment, the double-stranded nucleic add molecule may be easily recognized by Dicer because the nucleotides located at the 16^{th} or more positions from the end of the sense strand and the nucleotides in the antisense strand complementary thereto are not chemically modified.

The double-stranded nucleic acid molecule according to an embodiment or the double-stranded nudeic acid molecule included in the radial nucleic acid molecule according to an embodiment may be cleaved by Dicer to generate a cleaved double-stranded nucleic add. In an embodiment, the double-stranded nudeic acid cleaved by Dicer (or deaved product) may have an overhang 1 to 5 nt, 2 to 5 nt, 2 to 4 nt, 2 to 3nt, or 2 nt long on the 3′ end of the sense strand.

The cleaved double-stranded nucleic add (or cleaved product) can be loaded to an RNA-induced silencing complex (RISC) through the RISC-loading complex (RLC) mediated by Dicer and the human immunodeficiency virus transactivating response RNA-binding protein (TRBP) (step \textcircled{2})). In this process, in vertebrates such as humans, asymmetric sensing occurs in which the thermodynamically weak strand in the truncated double-stranded nucleic acid of TRBP is rearranged so that it can be recognized as a guide RNA by Ago2.

RISC is a ribonucleoprotein that recognizes and loads a double-stranded nucleic acid. to Ago2 (Argonaute 2), which is a protein responsible for the catalytic domain in the RISC, degrades the thermodynamically strong strand (passenger RNA) among the loaded double-stranded nucleic add while leaving the thermodynamically weak strand (guide RNA) (step \textcircled{3})). Since the antisense strand induded in the double-stranded nucleic acid molecule (or radial nucleic acid molecule) according to an embodiment is designed to be a thermodynamically weak strand, the sense strand is degraded with high efficiency and the antisense strand remains. The antisense strand recognizes the mRNA of a target gene (step \textcircled{4})) and complementarily binds thereto to form a dsRNA which is then degraded, whereby gene silencing can occur (step \textcircled{5})).

In one embodiment, the double-stranded nudeic add molecule (or the double-stranded nucleic acid molecule included in the radial nucleic acid molecule according to one embodiment) is administered to a subject or cells and degraded by Dicer into a fragment having an appropriate length (e.g., 19 to 30 nt, 20 to 30 nt, or 22 to 27 nt). The appropriate length may be “20 to 25” +2 nt (e.g., 19+2 nt, 20+2 nt, 21+2 nt, 22+2 nt, 23+2 nt, 24+2 nt, or 25+2 nt (i.e., the nucleic acid with a 2-nt overhang on the 3′ end of the sense strand).

Compared to a double-stranded nucleic acid in which all nucleotides are not chemically modified (control) or a double-stranded nucleic acid chemically modified by a conventional known method (e.g., alternating modification) and/or a C/U sequence-based modification method, the double-stranded nucleic acid molecule according to an embodiment (or the radial nucleic acid molecule according to an embodiment) allows the Dicer reaction rate to be similar or increased in vitro and/or in vivo. A double-stranded nucleic acid molecule according to an embodiment (or a radial nucleic acid molecule according to an embodiment) can be analyzed for Dicer cleavage (%) or Dicer reaction rate by a known method. According to an embodiment, the Dicer kinetic analysis can be conducted by mixing 10 pmoles of the double-stranded nucleic acid molecule (or radial nucleic acid molecule), recombinant human Dicer, a Dicer reaction buffer (Tris-HCl (pH 6.5-7.0) and NaCl), DEPC-treated deionized water (DW), and MgCl₂, incubating the mixture at 35-40° C. for 8-24 hours, measuring a Dincer substrate band thickness for each sample after electrophoresis, and calculating Dicer deavage (%). In an embodiment, Dicer can degrade the double-stranded nudeic acid molecule or the radial nucleic acid molecule according to an embodiment by 40 to 60% after reaction for one hour, by 60 to 80% after reaction for three hours, and by 90 to 100% after is reaction for 6 hours, as calculated by the method described above.

The double-stranded nucleic acid molecule (or the radial nucleic acid molecule) according to an embodiment may exhibit higher gene silendng activity in vitro and/or in vivo than double-stranded nucleic acids in which all nucleotides are not chemically modified or double-stranded nucleic acids chemically modified by a conventional known method (e.g., alternating modification) and/or a C/U sequence-based modification method.

As used herein, the term “small interfering RNA” (siRNA) refers to a short (e.g., 19 nt) double-stranded RNA (dsRNA) that mediates efficient gene silendng in a sequence-specific manner. A double-stranded nucleic acid molecule (or radial nucleic acid molecule) according to an embodiment is different from siRNA in that the former is a long double-stranded RNA and can serve as a Dicer substrate. It can be understood that a product resulting from the deavage of a double-stranded nucleic acid molecule (or radial nucleic acid molecule) according to an embodiment by Dicer is a kind of siRNA.

In a double-stranded nucleic acid molecule according to an embodiment (or in a is double-stranded nucleic acid molecule included in a radial nucleic add molecule according to an embodiment), the 3′ end of the sense strand and the 5′ end of the antisense strand may each be a blunt end.

In a double-stranded nucleic acid molecule according to an embodiment (or in a double-stranded nucleic acid molecule included in a radial nucleic add molecule according to an embodiment), the sense strand and/or the antisense strand may have an overhang 1 to 5 nt, 2 to 5 nt, 2 to 4 nt, 2 to 3 nt, or 2 nt long on either or both of the 3′ end and 5′ end. A double-stranded nucleic acid molecule according to an embodiment (or a radial nucleic acid molecule according to an embodiment) may have an overhang on the 3′ end of the antisense to strand and as such, can guarantee excellent accuracy of Dicer processing. The term “overhang” refers to a stretch of unpaired nucleotides in the end of a double-stranded nucleic add molecule.

In an embodiment, the nucleotides responsible for the overhang may be chemically unmodified.

In an embodiment, the double-stranded nucleic add molecule may be in the form of a hairpin or stem-loop consisting of a single-stranded nucleotide.

The sense strand may include the sequence of SEQ ID NO: 26, SEQ ID NO: 27, SEQ ID NO: 34, SEQ ID NO: 44, SEQ ID NO: 51, SEQ ID NO: 61, SEQ ID NO: 63, SEQ ID NO: 65, or SEQ ID NO: 67. The antisense strand may include the sequence of SEQ ID NO: 25, SEQ ID NO: 28, SEQ ID NO: 43, SEQ ID NO: 45, SEQ ID NO: 52, SEQ ID NO: 62, SEQ ID NO: 64, SEQ ID NO: 66, or SEQ ID NO: 68.

By the phrase “a sense strand, an antisense strand, or a polynucleotide strand indudes (comprises) a specific nucleotide sequence”, it is meant that a sense strand, an antisense strand, or a polynucleotide strand consists of or consists essentially of a specific nudeotide sequence or a specific amino acid sequence.

A sense strand included in a double-stranded nucleic acid molecule according to an embodiment may include the same sequence as all or part of a target gene (or mRNA of a target gene). The sense strand may consist of or consist essentially of a nucleic acid sequence having a homology (identity) of 70% or more, 80% or more, 85% or more, 90% or more, 91% or more, 92% or more, 93% or more, 94% or more, 95% or more, 96% or more, 97% or more, 98% or more, 98% or more, 99.5% or more, [symbol] 99.9% or more, or 100% with the nucleic acid sequence of a target gene.

An antisense strand included in a double-stranded nucleic add molecule according to an embodiment may include a sequence complementary to all or part of a target gene (or mRNA of a target gene). The antisense strand may consist of or consist essentially of a sequence 60% or more, 65% or more, 70% or more, 75% or more, 80% or more, 85% or more, 90% or more, 92% or more, 94% or more, 95% or more, 96% or more, 97% or more, 98% or more, 98.5% or more, 99% or more, 99.5% or more, 99.8% or more, 99.9% or more, or 100% complementary to all or part of a target gene (or mRNA of a target gene) and as such, the antisense strain can bind to (hybrid with) the target gene.

A double-stranded nucleic acid molecule (or radial nucleic acid molecule) according to an embodiment may be adapted to regulate the expression of a target gene, and specifically to downregulate (suppress or inhibit) the expression of a target gene into mRNA and/or protein. The target gene is a gene of interest to be regulated for mRNA and/or protein expression by the double-stranded nudeic add molecule and may be an endogenous gene or a transgene introduced into a cell by an expression vector.

In an embodiment, the target gene may be selected from a protein-encoding gene, a proto-oncogene, an oncogene, a tumor suppressor gene, and a cell signaling gene.

In an embodiment, the double-stranded nucleic acid molecule (or the radial nucleic acid molecule according to an embodiment) may have an 1050 value for the target gene of mg/kg or more, 0.005 mg/kg or more, 0.007 mg/kg or more, 0.009 mg/kg or more, 0.01 mg/kg or more, 0.05mg/kg or less, 0.03 mg/kg or less, 0.028 mg/kg or less, 0.025 mg/kg or less, 0.02 mg/kg or less, 0.015 mg/kg or less, 0.01 mg/kg or less, 0.001 to 0.045 mg/kg, 0.005 to 0.045 mg/kg, 0.007 to 0.045 mg/kg, 0.009 to 0.045 mg/kg, 0.01 to 0.045 mg/kg, 0.001 to mg/kg, 0.005 to 0.04 mg/kg, 0.007 to 0.04 mg/kg, 0.009 to 0.04 mg/kg, 0.01 to 0.04 mg/kg, 0.001 to 0.035 mg/kg, 0.005 to 0.035 mg/kg, 0.007 to 0.035 mg/kg, 0.009 to 0.035 mg/kg, 0.01 to 0.035 mg/kg, 0.001 to 0.03 mg/kg, 0.005 to 0.03 mg/kg, 0.007 to 0.03 mg/kg, to 0.03 mg/kg, or 0.01 to 0.03 mg/kg. Compared to siRNAs fora target gene, including double-stranded nucleic acid in which no nucleotides are chemically modified and double-stranded nucleic acids chemically modified by alternating modification and/or C/U sequence-based modification, the double-stranded nudeic acid molecule according to an embodiment (or the radial nucleic acid molecule according to an embodiment) may have a lower IC50 value for the same target gene by 1.1 times or more, 1.2 times or more, 1.3 times or more, 1.5 times or more, 2 times or more, 2.5 times or more, 3 times or more, 15 times or more, 12 times or more, 10 times or more, 8 times or more, 5 times or more, 1.1 to 15 times, 1.5 to 15 times, 2 to 15 times, 2.5 to 15 times, 3 to 15 times, 1.1 to 12 times, 1.5 to 12 times, 2 to 12 times, 2.5 to 12 times, 3 to 12 times, 1.1 to 10 times, 1.5 to 10 times, 2 to 10 times, 2.5 to 10 times, 3 to 10 times, 1.1 to 9 times, 1.5 to 9 times, 2 to 9 times, 2.5 to 9 times, 3 to 9 times, 1.1 to 8 times, 1.5 to 8 times, 2 to 8 times, 2.5 to 8 times, 3 to 8 times, 1.1 to 5 times, 1.5 to 5 times, 2 to 5 times, 2.5 to 5 times, or 3 to 5 times.

The double-stranded nudeic acid molecule according to an embodiment may further include a polynudeotide 1 to 30 nt, 2 to 30n, 5 to 30 nt, 10 to 30 nt, 20 to 30 nt, 25 to 30 nt, 1 to 27 nt, 2 to 27 nt, 5 to 27 nt, 10 to 27 nt, 20 to 27 nt, 25 to 27 nt, 1 to 25 nt, 2 to 25 nt, 5 to nt, 10 to 25 nt, or 20 to 25 nt long at the 3′ end and/or 5′ end of the sense strand and/or the antisense strand. All or part of the polynucleotides additionally extended from the sense strand and the antisense strand may or may not complementarily bind to each other. Even if additionally including a polynucleotide with such a predetermined length, the double-stranded nucleic add molecule according to an embodiment may have an unaffected activity (e.g., gene silencing activity).

In an embodiment, the double-stranded nucleic acid molecule may additionally include:

a polynucleotide with such a length (e.g., 1 to 30 nt) at the 3′ end of the sense strand, and

a polynucleotide with such a length (e.g., 1 to 40 nt) at the 5′ end of the antisense strand. The nucleotides extending from the 3′ end of the sense strand may not complementarily bind to those extending from the 5′ end of the antisense strand. In this case, the double-stranded nucleic acid molecule according to an embodiment may be a flanking-end dsRNA. The flanking-end double-stranded nudeic acid molecule is exemplified as shown in FIG. 9.

Another aspect provides a radial nudeic acid molecule including the double-stranded nucleic acid molecule. A radial nucleic acid molecule according to an embodiment may include 2 to 5, 2 to 4, or 2 to 3 double-stranded nucleic add molecules. The double-stranded io nucleic acid molecule included in a radial nucleic acid molecule according to an embodiment is as described above.

The radial nucleic acid molecule indudes K arms (e.g., K is an integer 2≤K≤5, 2≤K≤4, or 2≤K≤3) extending radially, wherein each arm may indude the double-stranded nudeic acid molecule and a polynudeotide extending from the double-stranded nudeic acid molecule to is the central and/or terminal portion thereof. In an embodiment, each arm may further include a polynudeotide extending to a length of 1 to 30 nt, 2 to 30 n, 5 to 30 nt, 10 to 30 nt, 20 to 30 nt, 25 to 30 nt, 1 to 27 nt, 2 to 27 nt, 5 to 27 nt, 10 to 27 nt, 20 to 27 nt, 25 to 27 nt, 1 to 25 nt, 2 to 25 nt, 5 to 25 nt, 10 to 25 nt, or 20 to 25nt from the double-stranded nucleic acid molecule in the direction toward the central and/or terminal portion. The double-stranded nucleic acid molecules induded in the arms may be identical to or different from each other. Individual arms may be open or may be closed as in a stem-loop structure (or hairpin structure).

As used herein, the term “radial” refer to pertaining to a structure having a form in which K arms are radially extended (i.e., a form extending in all 2D or 3D directions from a central point, such as in a spider's web or spokes). In this regard, K may be an integer of 2≤K≤5, 2≤K≤4, or 2≤K≤3. For example, when K is 3, the nudeic add molecule exhibits a Y-shaped structure. For K being 4, the nucleic add molecule exhibits a +-shaped structure. Given 5, K allows the nudeic acid molecule to represent a *-like structure.

With such a structure, the radial nucleic add molecule including K radially extending arms exhibits high resistance to nucleases (e.g., endonucleases, exonucleases) in vivo, resulting in an increase in blood stability. In addition, the size of the nucleic add molecule can be controlled by adjusting the length of each arm and the number of arms, thereby exhibiting an EPR (Enhanced Permeability and Retention) effect. The EPR effect means that molecules having a specific size tend to accumulate in tumor tissues rather than normal tissues. It is known that the types of organs in which nudeic add molecules accumulate are different depending on the size of the nudeic acid molecules. A target organ in which nucleic add molecules are accumulated can be set by adjusting the length of each arm and/or number of arms.

The radial nucleic acid molecule according to an embodiment may include the double-stranded nucleic acid molecule. Serving as a substrate of Dicer, each of the double-stranded nucleic acid molecules can be cleaved by Dicer to give a cleaved product that can exhibit silencing activity against a target gene. The radial nucleic add molecule according to an embodiment may indude K arms extending radially and the double-stranded nucleic acid is molecule in each arm and can be prepared into cleaved products that exhibit silencing activity against as many as K genes (1 to K, both inclusive). The radial nucleic acid molecule includes a plurality of double-stranded nucleic adds having identical or different sequences within one molecule to selectively, concurrently, and/or efficiently modulate (e.g., downregulate) the expression of a plurality of genes.

The double-stranded nucleic acid molecule responsible for the arm includes a sense strand and an antisense strand bearing a chemically modified nucleotide at a specific position, so that the radial nucleic acid molecule may maintain, improve, or reduce at least one from the group consisting of the following features (1) to (8), compared to a double-stranded nucleic acid the nudeotides of which are all chemically unmodified or a double-stranded nucleic add chemically modified by an already known method (for example, alternating modification and/or C/U sequence-based modification):
(1) maintenance and/or increase of interaction between the nucleic acid induding K radially extending arms and Dicer in vivo and in vitro (for example, maintenance and/or increase of the rate of deavage of double-stranded nucleic acids by Dicer);
(2) maintenance and/or increase of target gene silencing activity in vitro;
(3) increase in target gene silencing activity in vivo;
(4) decrease in off-target effect;
(5) decrease in degradation by nuclease (e.g., RNase) in vivo;
(6) increase in intracellular uptake;
(7) increase in stability in vitro and/or in vivo (e.g., increase in serum stability); and/or
(8) decrease in immune response (e.g., induction of TLR-mediated immune response within endosomes by the double-stranded nudeic add molecule or induction of PKR-mediated immune response by the double-stranded nucleic acid molecule released into the cytosol).

The radial nucleic acid molecule including K arms (e.g., an integer of 2≤K≤3) extending radially includes a plurality of different double-stranded nucleic acids in one molecule, thereby increasing in binding affinity for Dicer.

The nucleotide sequences of the double-stranded nucleic acid molecules induded in individual arms of the radial nucleic acid molecule may be identical (having gene silendng activity for the same target gene) or different from each other (having gene silendng activity for different target genes). In an embodiment, when the nucleotide sequences of double-stranded nucleic acid molecules included in the arms of a Y-shaped radial nucleic acid molecule are all different from one another, three different cleaved products (by Dicer deavage) can be prepared ata ratio (e.g., molar ratio) of 1:1:1 from the nucleic acid molecule. In one embodiment, the double-stranded nudeic acid molecules included in the three stems of a Y-shaped radial nucleic acid molecule have identical nucleotide sequences between two stems and a different nucleotide sequence for the other stem, the deaved products that can be prepared from the nucleic add molecules may be two different products present at a ratio (e.g., molar ratio) of 2:1. According to an embodiment, as described above, ratios of possible deaved products (capable of exhibiting gene silencing activity) that are prepared from the nucleic add molecule can be arbitrarily controlled by adjusting the types (sequences) of the double-stranded nudeic acid molecule included in the radial nucleic acid molecule.

The radial nucleic acid molecule may be endogenously cleaved by Dicer, and according to an embodiment, the double-stranded nucleic acid molecule included in the arm of the radial nucleic acid molecule may be endogenously cleaved by Dicer. In an embodiment, the efficiency of the double-stranded nucleic acid molecule having gene silendng activity can be increased by controlling the thermodynamic stability of the sequence of the double-stranded nucleic acid molecule included in the stem of each arm.

In one embodiment, when the radial nucleic acid molecule is administered to a cell, tissue, and/or subject, the double-stranded nucleic acid molecule contained in each arm is cut to an appropriate length by Dicer and then loaded into the RISC, and Ago2 (Argonaute 2) responsible for a catalytic domain in the RISC degrades the thermodynamically strong strand from the sequence of the loaded double-stranded nucleic acid molecule and leaves a thermodynamically weak strand. Therefore, the sequence of the antisense strand to the target gene may be designed to be a thermodynamically weak strand in the sequence of the is double-stranded nudeic acid molecule included in the arm, whereby the gene silendng effect (efficiency) of the nucleic add molecule can be increased.

The double-stranded nucleic acid molecule induded in the radial nucleic acid molecule is as described in the foregoing.

A radial nucleic acid molecule according to an embodiment may include two double-stranded nudeic acid molecules (e.g., a first double-stranded nudeic acid molecule and a second double-stranded nucleic acid molecule)
wherein,
the 3′ end of the sense strand in the first double-stranded nucleic add molecule is linked to the 5′ end of the antisense strand in the second double-stranded nudeic acid molecule;
the 5′ end of the antisense strand in the first double-stranded nucleic acid molecule is linked to the 3′ end of the sense strand in the second double-stranded nudeic acid molecule; or
the 3′ end of the sense strand in the first double-stranded nucleic add molecule is linked to the 5′ end of the antisense strand in the second double-stranded nudeic acid molecule and the 5′ end of the antisense strand in the first double-stranded nucleic acid molecule is linked to the 3′ end of the sense strand in the second double-stranded nucleic acid molecule.

A radial nucleic acid molecule according to an embodiment may include three double-stranded nucleic add molecules (e.g., a first double-stranded nucleic acid molecule, a second double-stranded nucleic acid molecule, and a third double-stranded nucleic acid molecule) and exhibit at least two of the following features (i) to (iii):
(i) linkage of the 3′ end of the sense strand in the first double-stranded nucleic acid molecule to the 5′ end of the antisense strand in the second double-stranded nucleic acid molecule;
(ii) linkage of the 5′ end of the antisense strand in the first double-stranded nucleic acid molecule to the 3′ end of the sense strand in the third double-stranded nucleic acid molecule; is and
(iii) linkage of the 3′ end of the sense strand in the second double-stranded nucleic acid molecule to the 5′ end of the antisense strand in the third double-stranded nucleic acid molecule.

According to an embodiment, the radial nudeic acid molecule including three double-stranded nucleic add molecules and exhibiting two selected from the group consisting of the features (i) to (iii) may have a Y-shaped structure with one nick in the center. According to an embodiment, the radial nucleic acid molecule including three double-stranded nucleic acid molecules and exhibiting all of the features (i) to (iii) may have a Y-shaped structure with no nicks therein. FIG. 9 depicts a Y-shaped radial nudeic add molecule with a nick and a Y-shaped radial nucleic add molecule with no nicks (free of nicks) according to an embodiment.

As used herein, the linkage of the 3′ end of the sense strand in an n-th double-stranded nucleic acid molecule to the 5′ end of the antisense strand in an m-th double-stranded nucleic acid molecule may mean the connection between the 3′ end of the sense strand and the 5′ end of the antisense strand vie a phosphodiester bond.

In an embodiment, the first nucleotide from the 5′ end of the sense strand in a double-stranded nudeic acid molecule (or a double-stranded nudeic acid molecule included in a radial nucleic acid molecule according to an embodiment) may be G or C.

In an embodiment, the 20^{th} nucleotide from the 5′ end of the sense strand in a double-stranded nudeic acid molecule (or a double-stranded nudeic acid molecule included in a radial nucleic acid molecule according to an embodiment) may be A or U(T).

In an embodiment, 4 or more of the 14^{th} to 21^{st} nucleotides from the 5′ end of the sense strand in a double-stranded nucleic acid molecule (or a double-stranded nucleic acid molecule included in a radial nucleic acid molecule according to an embodiment) are U(T) or A.

Another aspect may provide a composition including the double-stranded nudeic acid molecule and/or the radial nucleic acid molecule for inhibiting gene expression.

The gene may be selected from protein-encoding genes, proto-oncogenes, oncogenes, tumor suppressor genes, and cell signaling genes.

In an embodiment, the composition for inhibiting gene expression may further include a carrier. The carrier may be at least one selected from the group consisting of lipid molecules, liposomes, micelles, cationic lipids, cationic polymers, ligand conjugates, and cationic metals.

In an embodiment, the double-stranded nucleic acid molecule and/or the radial nucleic acid molecule can be delivered be being added directly or complexed with cationic lipids or packaged within liposomes. For example, the nucleic acid molecule can be administered to cells and/or subjects by a variety of methods known to those of skill in the art, including encapsulation in liposomes, by ionophoresis, or by incorporation into other vehides, such as biodegradable polymers, hydrogels, cyclodextrins, poly(lactic-co-glycolic)acid (PLGA) and PLCA microspheres, biodegradable nanocapsules, and bioadhesive microspheres.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains a nucleic acid molecule. The lipophilic material isolates the aqueous interior from an aqueous exterior, which typically does not indude the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the nucleic acid molecule are delivered into the cell where the nucleic acid molecule can specifically bind to a target gene and can mediate RNAi. In some cases, the liposomes are also specifically targeted to direct the siRNA to particular cell types.

“Micelles” are defined herein as a particular type of molecular assembly in which amphipathic molecules are arranged in a spherical structure such that all the hydrophobic is portions of the molecules are directed inward, leaving the hydrophilic portions in contact with the surrounding aqueous phase. The converse arrangement exists if the environment is hydrophobic.

Another aspect provides a method for inhibiting expression of a target gene, the method including a step of administering to a subject an effective amount of the double-stranded nucleic acid molecule, the radial nucleic acid molecule, and/or the composition for inhibiting gene expression. The method for inhibiting expression of a target gene may further include the step of identifying a subject in need of inhibiting expression of the gene before the administering step.

In an embodiment, the method for inhibiting expression of a target gene may be adapted to downregulate the expression of the target gene in a target cell in vitro.

Another aspect is to provide a use of the double-stranded nucleic acid molecule and/or the radial nucleic acid molecule for prevention or treatment of a disease.

Another aspect is to provide a pharmaceutical composition including the double-stranded nucleic acid molecule and/or the radial nudeic acid molecule for prevention or treatment of a disease. The double-stranded nucleic acid molecule may further include a pharmaceutically acceptable carrier in addition to the nucleic acid molecule induding K arms extended in a radial direction.

Another aspect provides a method for preventing or treating a disease, the method including a step of administering a pharmaceutically effective amount of the pharmaceutical composition to a subject. The method may further include a step of identifying an individual in need of preventing or treating a disease before the administering step.

The disease may include a genetic disease and/or a non-genetic disease.

The disease may be at least one selected from the group consisting of cancer, proliferative disease, digestive disease, kidney disease, neurological disease, mental disease, blood and tumor disease, cardiovascular disease, respiratory disease, endocrine disease, infectious disease, musculoskeletal disease, gynecological disease, genitourinary disease, skin disease, and ophthalmic disease, but is not limited thereto.

The cancer may be a solid cancer or a blood cancer and for example, may be at least one selected from the group consisting of squamous cell carcinoma, small cell lung cancer, non-small cell lung cancer, adenocarcinoma of the lung, squamous cell carcinoma of the lung, peritoneal cancer, skin cancer, skin or intraocular melanoma; rectal cancer, perianal cancer, esophageal cancer, small intestine cancer, endocrine adenocarcinoma, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, cervical cancer, ovarian cancer, liver cancer, bladder cancer, breast cancer, colon cancer, colorectal cancer, endometrial or uterine cancer, salivary gland cancer, kidney cancer, prostate cancer, vulvar cancer, thyroid cancer, head and neck cancer, brain cancer, and osteosarcoma, but with no limitations thereto.

The proliferative disease may be at least one selected from the group consisting of myeloproliferative disease (MPD), primary myelofibrosis, chronic myeloproliferative disease (e.g., polycythemia vera), thrombocythemia vera, idiopathic thrombocythemia (essential thrombocythemia), chronic myeloid leukemia, and/or idiopathic myelofibrosis, and aplastic anemia (e.g., severe aplastic anemia), but with no limitations thereto.

The digestive diseases include peptic ulcer disease (PUD), gastroesophageal reflux disease, constipation, diarrhea, irritable bowel syndrome, nausea and vomiting, inflammatory bowel disease, pancreatitis, liver cirrhosis and complications, viral hepatitis, and drug-induced liver injury, but with no limitations thereto.

The kidney disease may be at least one selected from the group consisting of fluid and electrolyte imbalance (fluid and electrolyte disorders), add-base disorders, drug-induced kidney disease, renal impairment, acute kidney injury, and chronic kidney disease, but with no limitations thereto.

The neurological disease may be one or more selected from the group consisting of headache, epilepsy, Alzheimer's disease, Parkinson's disease, and the like, but is not limited thereto.

The mental disorder may be one or more selected from the group consisting of major is depressive disorder, schizophrenia, generalized anxiety disorder, panic disorder, bipolar disorder, attention deficit hyperactivity disorder, alcohol, nicotine and caffeine addiction, sleep disorder, and eating disorders, but with no limitations thereto.

The blood and tumor disease may be one or more selected from the group consisting of anemias, lung cancer, gastric cancer, colorectal cancer, breast cancer, gynecologic cancer, prostate cancer, leukemias, and lymphomas, but is not limited thereto.

The cardiovascular disease may be at least one selected from the group consisting of hypertension, heart failure, ischemic heart diseases, acute coronary syndrome (ACS), arrhythmias, dyslipidemia, stroke, venous thromboembolism, peripheral arterial disease, and hypovolemic shock, but with no limitations thereto.

The respiratory disease may be at least one selected from the group consisting of asthma, chronic obstructive pulmonary disease, and allergic rhinitis, but with no limitations thereto.

The endocrine disease may be at least one selected from the group consisting of diabetes mellitus, thyroid disorder), and pituitary & adrenal gland disorders, but with no limitations thereto.

The infectious disease may be at least one selected from the group consisting of upper respiratory tract infection, pneumonia, urinary tract infection, tuberculosis, meningitis, gastrointestinal/intraabdominal infections, skin and soft tissue infection, superficial fungal

Infections/deep mycoses, sepsis, sexually transmitted infection (STI), but with no limitations thereto.

The musculoskeletal disease may be at least one selected from the group consisting of osteoarthritis, rheumatoid arthritis, osteoporosis, gout, and hyperuricemia, but with no limitations thereto.

The gynecological disease may be at least one selected from the group consisting of drug use in pregnancy and lactation, menopause, and urinary incontinence, but with no limitations thereto.

The genitourinary disease may be at least one selected from the group consisting of benign prostatic hyperplasia and prostatitis, but with no limitations thereto.

The skin disease may be at least one selected from the group consisting of atopic dermatitis and psoriasis, but with no limitations thereto.

The ophthalmic disease may be glaucoma, but is not limited thereto.

As used herein, the term “administration” refers to introducing a prescribed substance into a patient via any appropriate method, and a route of administration of the conjugate may be any common methods as long as a drug reaches a target tissue. Particularly, the pharmaceutical composition may be administered via intraperitoneal administration, intravenous administration, intramuscular administration, subcutaneous administration, intradermal administration, oral administration, topical administration, intranasal administration, intrapulmonary administration, and intrarectal administration, but is not limited thereto. However, because a peptide is digested when administered orally, it is preferred that a composition for oral administration is formulated so that the active substance is coated or protected against degradation in stomach. Preferably, it may be administered in the form of injections. Additionally, a pharmaceutical composition may be administered by any device which can transport active substances to target cells.

In the pharmaceutical composition or method according to an embodiment, the pharmaceutical composition or the composition including the double-stranded nucleic acid molecule, and/or the radial nudeic acid molecule may be provided together with at least one additive selected from the group consisting of a carrier, a diluent, and an excipient.

The pharmaceutically acceptable carrier can be typically used for formulating the pharmaceutical composition into preparations and may be at least one selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil, but with no limitations thereto. In addition to the above components, the pharmaceutical composition may include at least one selected from the group consisting of diluents, excipients, lubricants, humectants, sweeteners, flavoring agents, emulsifiers, suspending agents, and preservatives, which are commonly used for preparing pharmaceutical compositions.

The composition (pharmaceutical composition or composition for inhibiting gene expression) may be administered orally or parenterally. Parenteral administration may be achieved by intravenous injection, subcutaneous injection, intramuscular injection, intraperitoneal injection, endothelial administration, topical administration, intranasal administration, intrapulmonary administration, rectal administration, etc. Because a protein or peptide is digested when administered orally, a composition for oral administration is formulated so that the active substance is coated or protected against degradation in stomach. Additionally, a pharmaceutical composition may be administered by any device which can transport active substances to target cells.

A suitable dose of the pharmaceutical composition may vary depending on pharmaceutical formulation methods, administration modes, the patient's age, body weight, sex, severity of diseases, diet, administration time, administration route, an excretion rate, and sensitivity.

The pharmaceutical composition may be administered at a daily dose of 0.1 to 1000 mg/kg or 0.1 to 200 mg/kg for adults. For instance, a composition containing 0.1 to 100 nmoles of the double-stranded molecule may be administered at a dose of 0.1 to 1000 mg/kg, 1 to 500 mg/kg, or 1 to 100 mg/kg. Administration may be performed once daily or in several divided doses a day. The daily dose may be formulated into a unit dosage form or distributed into separate dosage forms, or may be included within a multiple-dose container.

As used herein, “pharmaceutically effective amount” or “effective amount” means a dose at which the active ingredient can exert a desired effect (i.e., effect of inhibiting the expression of a target gene or preventing and/or treating cancer or proliferative disease) and may vary depending on various factors including formulation methods, administration modes, the patient's age, body weight, sex, severity of diseases, diet, administration time, administration route, an excretion rate, and sensitivity.

According to the conventional techniques known to those skilled in the art, the pharmaceutical composition may be formulated with pharmaceutically acceptable carrier and/or vehicle, finally providing several forms including a unit dose form and a multi-dose form. In this regard, the formulation may be in the form of a solution in an oil or aqueous medium, a suspension, a syrup, an emulsion, an extract, a powder, a granule, a tablet, or a capsule, and may further include a dispersant or a stabilizer.

In addition, the pharmaceutical composition may be administered as an independent drug or can be co-administered with other drugs. In the case of co-treatment, it can be administered sequentially or simultaneously with conventional therapeutic agents.

The patient (subject) to which the gene expression-inhibiting composition or pharmaceutical composition is administered or to which the gene expression-inhibiting method or the preventing or treating method is applied may be mammals including primates such as humans, monkeys, etc. and rodents such as rats, mice, etc., or cells or tissues isolated from mammals, or a culture thereof, but is not limited thereto.

Another aspect provides a method for producing a nudeic acid molecule (e.g., a nucleic acid molecule with increased in vivo stability and/or increased gene silencing activity), the method including the steps of: synthesizing x polynucleotide strands (i.e., x is an integer of 1 to 5), each bearing a chemically modified nucleotide at a specific position; and hybridizing the x strands. The nucleic acid molecule produced by the method may retain the aforementioned characteristics of the double-stranded nucleic acid molecule or the radial nucleic acid molecule.

The polynucleotide strand prepared in the synthesizing step indudes a sense region ((n-S (sense) region) including a sequence identical to all or part of the sequence of the n-th target gene and an antisense region (m-AS (antisense) region) including a sequence to complementary to all or part of the m-th target gene, or a combination thereof (the n-S region and the m-AS region). In this regard, n, x, and m may be positive integers, for example, each be an integer of 1 to 5.

The “sense region” is a polynucleotide region induding the same nucleic acid sequence as all or part of a target gene, and may have the aforementioned characteristics of the “sense strand”. The sense region may indude or consist essentially of a nucleic acid sequence having a homology (or identity) of 70% or more, 80% or more, 85% or more, 90% or more, 91% or more, 92% or more, 93% or more, 94% or more, 95% or more, 96% or more, 97% or more, 98% or more, 98% or more, 99.5% or more, 99.9% or more, or 100% with the nucleic add sequence (base sequence) of a target gene (e.g., mRNA of a target gene).

The “antisense region” is a polynucleotide region including a nudeic acid sequence that is substantially or 100% complementary to all or part of a target gene, and may have the aforementioned characteristics of the “antisense strand”. The antisense region may indude or consist essentially of a nucleic acid sequence 60% or more, 65% or more, 70% or more, 75% or more, 80% or more, 85% or more, 90% or more, 92% or more, 94% or more, 95% or more, 96% or more, 97% or more, 98% or more, 98.5% or more, 99% or more, 99.5% or more, 99.8% or more, 99.9% or more, or 100% complementary to all or part of a target gene to be bound (e.g., mRNA of a target gene) so that the antisense region can bind to (hybridize with) the target gene.

In an embodiment, the antisense region may complementarily bind to a sense region induded in another (separate) polynucleotide strand and, specifically, may include or consist essentially of a nucleic acid sequence 60% or more, 65 or more, 70% or more, 75% or more, 80% or more, 85% or more, 90% or more, 92% or more, 94% or more, 95% or more, 96% or more, 97% or more, 98% or more, 98.5% or more , 99% or more, 99.5% or more, 99.8% or more, 99.9% or more, or 100% complementary to a nudeic acid sequence of a sense region included in the separate polynudeotide strand.

In an embodiment, the sense region may be a single-stranded polynucleotide 19 to 70 nt (nucleotide), 20 to 70 nt, 21 to 70 nt, 22 to 70 nt, 23 to 70 nt, 25 to 70 nt, 19 to 66 nt, 20 to 66 nt, 21 to 66 nt, 22 to 66 nt, 23 to 66 nt, 25 to 66 nt, 19 to 60 nt, 20 to 60 nt, 21 to 60 nt, 22 to 60 nt, 23 to 60 nt, 25 to 60 nt, 19 to 55 nt, 20 to 55 nt, 21 to 55 nt, 22 to 55 nt, 23 to 55 nt, 25 to 55 nt, 19 to 52 nt, 20 to 52 nt, 21 to 52 nt, 22 to 52 nt, 23 to 52 nt, 25 to 52 nt, 19 to nt, 20 to 50 nt, 21 to 50 nt, 22 to 50 nt, 23 to 50 nt, 25 to 50 nt, 19 to 45 nt, 20 to 45 nt, 21 to 45 nt, 22 to 45 nt, 23 to 45 nt, 25 to 45 nt, 19 to 40 nt, 20 to 40 nt, 21 to 40 nt, 22 to 40 nt, 23 to 40 nt, 25 to 40 nt, 19 to 38 nt, 20 to 38 nt, 21 to 38 nt, 22 to 38 nt, 23 to 38 nt, 25 to 38 nt, 19 to 36 nt, 20 to 36 nt, 21 to 36 nt, 22 to 36 nt, 23 to 36 nt, 25 to 36 nt, 19 to 35 nt, 20 to nt, 21 to 35 nt, 22 to 35 nt, 23 to 35 nt, 25 to 35 nt, 19 to 30 nt, 20 to 30 nt, 21 to 30 nt, 22 to 30 nt, 23 to 30 nt, 25 to 30 nt, 19 to 28 nt, 20 to 28 nt, 21 to 28 nt, 22 to 28 nt, 23 to 28 nt, 25 to 28 nt, 19 to 25 nt, 20 to 25 nt, 21 to 25 nt, 22 to 25 nt, 23 to 25 nt, or 25 nt in length.

In an embodiment, the antisense region may be a single-stranded polynudeotide 20 to 70 nt, 21 to 70 nt, 22 to 70 nt, 23 to 70 nt, 25 to 70 nt, 27 to 70 nt, 20 to 66 nt, 21 to 66 nt, 22 to 66 nt, 23 to 66 nt, 25 to 66 nt, 27 to 66 nt, 20 to 60 nt, 21 to 60 nt, 22 to 60 nt, 23 to 60 nt, 25 to 60 nt, 27 to 60 nt, 20 to 55 nt, 21 to 55 nt, 22 to 55 nt, 23 to 55 nt, 25 to 55 nt, 27 to 55 nt, 20 to 52 nt, 21 to 52 nt, 22 to 52 nt, 23 to 52 nt, 25 to 52 nt, 27 to 52 nt, 20 to 50 nt, 21 to 50 nt, 22 to 50 nt, 23 to 50 nt, 25 to 50 nt, 27 to 50 nt, 20 to 45 nt, 21 to 45 nt, 22 to 45 nt, 23 to 45 nt, 25 to 45 nt, 27 to 45 nt, 20 to 40 nt, 21 to 40 nt, 22 to 40 nt, 23 to 40 nt, 25 to 40 nt, 27 to 40 nt, 20 to 38 nt, 21 to 38 nt, 22 to 38 nt, 23 to 38 nt, 25 to 38 nt, 27 to 38 nt, 20 to 36 nt, 21 to 36 nt, 22 to 36 nt, 23 to 36 nt, 25 to 36 nt, 27 to 36 nt, 20 to 35 nt, 21 to 35 nt, 22 to 35 nt, 23 to 35 nt, 25 to 35 nt, 27 to 35 nt, 20 to 30 nt, 21 to 30 nt, 22 to 30 nt, 23 to 30 nt, to 30 nt, 27 to 30 nt, 20 to 27 nt, 21 to 27 nt, 22 to 27 nt, 23 to 27 nt, 25 to 27 nt, or 27 nt in length.

The x polynucleotide strands synthesized in the synthesizing step (the 1^{st} strand, the 2^{nd} strand . . . the x-th strand, for example, x is an integer of 1)(5) may be designed to include a chemically modified nucleotide at a specific position. The sense region may indude a chemically modified nucleotide at one or more (e.g., one or more, two or more, three or more, all four) selected from the group consisting of 4^{th}, 5^{th}, 7^{th} and 14^{th} positions from the 5′ end.

The antisense region may include a chemically modified nucleotide at a position complementary to the nucleotide present at at least one (e.g., one or more, two or more, three or more, four or more, five or more, six or more, seven or more, or all of eight) selected from the group consisting of 2^{nd}, 3^{rd}, 6^{th}, 8^{th} and 10^{th} to 13^{th} positions from the 5′ end of the sense region of another (separate) polynucleotide strand (or at 8^{th} to 11^{th}, 13^{th}, 15^{th}, 18^{th}, and 19^{th} positions from the 5′ end of the antisense strand in the cleaved product). In an embodiment, the (n-S) region may include a chemically modified or unmodified nucleotide at the 1^{st} position from the 5′ end. The chemical modification is the same as described above, and specifically, the chemically modified nucleotide may include a sugar moiety modified with be at least one selected from the group consisting of 2′-O-methyl, 2′-methoxyethoxy, 2′-fluoro, 2′-allyl, 2′-O-[2-(methylamino)-2-oxoethyl], 4′-thio, 4′-CH2-O-2′-bridge, 4′-(CH2)2-O-2′-bridge, 2′-LNA, 2′-amino, and 2′-O-(N-methylcarbamate).

In an embodiment, the sense region may include a chemically unmodified nucleotide at one or more selected from the group consisting of 2^{nd}, 3^{rd}, 6^{th}, 8^{th} to 13^{th}, and 15^{th} or higher (e.g., 15^{th} to 35^{th} or 15^{th} to 25^{th}) from the 5′ end.

In an embodiment, the antisense region may include a chemically unmodified nucleotide at one or more positions selected from the group consisting of 1^{st}, 4^{th}, 5^{th}, 7^{th}, 9^{th}, and 14^{th} or higher positions (e.g., 14^{th} to 35^{th}) from the 5′ end of the sense region complementary thereto in another (separate) polynucleotide strand or at one or more positions selected from the group consisting of 7^{th} or lower (e.g., 1^{st} to 7^{th}) 12^{th}, 14^{th}, 16^{th}, 17^{th}, and 20^{th} or higher positions (e.g., 20^{th} to 22^{nd}) from the 5′ end of the antisense strand in the product generated through Dicer cleavage.

The step of synthesizing a polynucleotide strand may be performed by a method known in the art, for example, a method using nucleoside phosphoramidites or a ‘phosphite triester’ method, developed by Koster, using b-cyanoethyl phosphoramidite to form phosphodiester bonds accounting for the backbone of a DNA structure.

Among the x polynucleotide strands prepared in the synthesizing step, the sense region included in one strand may complementarily bind to the antisense region of another strand, and the x polynucleotide strands may be designed such that at least one pair of polynucleotide strands may complementarily bind to each other.

In the producing method according to an embodiment, when x is 2,
the synthesizing step may be designed to synthesize:
a first polynucleotide strand including a sense region (1-S region) that has the same sequence as all or part of a first target gene and/or an antisense region (2-AS region) that has a sequence complementary to all or part of a second target gene; and
a second polynucleotide strand including a sense region (2-S region) that has the same sequence as all or part of the second target gene and/or an antisense region (1-AS region) that has a sequence complementary to all or part of the first target gene. In an embodiment, a nucleic acid molecule prepared by synthesizing a first polynucleotide strand including the (1-S) region and a second polynucleotide strand including the (1-AS) region and hybridizing the strands with each other may have a flanking end. The flanking-end nucleic add molecule is exemplified as depicted in FIG. 9.

In the producing method according to an embodiment, when x is 3,
the synthesizing step may be designed to synthesize:
a first polynucleotide strand including a sense region (1-S region) that has the same sequence as all or part of a first target gene and/or an antisense region (2-AS region) that has a sequence complementary to all or part of a second target gene;
a second polynucleotide strand including a sense region (2-S region) that has the same sequence as all or part of the second target gene and/or an antisense region (3-AS region) that has a sequence complementary to all or part of a third target gene; and
a third polynudeotide strand including a sense region (3-S region) that has the same sequence as all or part of a third target gene and/or an antisense region (1-AS region) that has a sequence complementary to all or part of the first target gene.

In an embodiment, a nucleic acid molecule prepared by synthesizing a first polynucleotide strand including the (1-S) region and a second polynucleotide strand including the (2-AS) region, a second polynucleotide strand induding the (2-S) region and the (3-AS) region, and a third polynucleotide sequence including the (3-S) region and the (1-AS) region and hybridizing the strands may be a Y-shaped radial nucleic acid molecule. The Y-shaped radial nucleic acid molecule is exemplified as shown in FIG. 9.

In the producing method according to an embodiment, when x is 4,
the synthesizing step may be designed to synthesize:
is a first polynucleotide strand including a sense region (1-S region) that has the same sequence as all or part of a first target gene and/or an antisense region (2-AS region) that has a sequence complementary to all or part of a second target gene;
a second polynucleotide strand including a sense region (2-S region) that has the same sequence as all or part of the second target gene and/or an antisense region (3-AS region) that has a sequence complementary to all or part of a third target gene;
a third polynudeotide strand including a sense region (3-S region) that has the same sequence as all or part of the third target gene and/or an antisense region (4-AS region) that has a sequence complementary to all or part of a fourth target gene; and
a fourth polynucleotide strand including a sense region (4-S region) that has the same sequence as all or part of the fourth target gene and/or an antisense region (1-AS region) that has a sequence complementary to all or part of the first target gene.

In an embodiment, the first target gene and the fourth target gene may be identical, and a nucleic acid molecule prepared by synthesizing a first polynucleotide strand induding the (1-S) region and the (2-AS) region, a second polynucleotide strand including the (2-S) region and the (3-AS) region, a third polynucleotide strand induding the (3-S) region, and a fourth polynucleotide sequence including the (4-AS) capable of complementarily binding to the (1-S) region and hybridizing the first to the fourth polynudeotide strands may be a Y-shaped radial nucleic acid molecule with a nick at the center thereof. The Y-shaped radial nucleic acid molecule with a nick is exemplified as depicted in FIG. 9.

The producing method according to an embodiment may include a step of hybridizing x polynucleotide strands prepared in the synthesizing step.

The hybridizing step may be performed by a conventional method, and according to an embodiment, the hybridization may be achieved through a reaction in a condition where the temperature is reduced from 70 to 120° C., 80 to 110° C., 90 to 110° C., or 90 to 100° C. to 0 to 30° C., 0 to 25° C., 0 to 20° C., 1 to 15° C., or 1 to 10° C. The temperature reduction in the hybridizing step may be conducted at a rate of −0.01 to 30° C./s, −0.05 to 20° C./s, —0.1 to 15° C./s, or −0.1 to 10° C./s.

The hybridization step may be performed under conditions known to those skilled in the art (see, for example, Sambrook et al., Molecular Cloning: A Laboratory Manual, 2^{nd} Ed., Cold Spring Harbor Laboratory Press, Cold Spring Harbor, N.Y. (1989)). In an embodiment, a stringent hybridization condition includes incubating a solution containing 50% formamide, (150 mM NaCl, 15 mM trisodium citrate), 50 mM sodium phosphate (pH 7.6), 5× Denhardt's solution, 10% dextran sulfate, and 20 μg/mL polynucleotide strands overnight at 42° C. and then washing the filter in 0.1×SSC at about 65° C.

Another aspect may provide a method for producing a double-stranded nucleic acid,
the method induding a step of preparing a double-stranded nucleic acid molecule including:
a sense strand 19 to 36 nt in length and an antisense strand 21 to 38 nt in length and having a sequence complementary to the sense strand,
the sense strand and the antisense strand each bearing a chemically modified nucleotide at one or more of the following positions:
(1) one or more positions on the sense strand, selected from 1^{st}, 4^{th}, 5^{th}, 7^{th} and 14^{th} positions from the 5′ end of the sense strand; and
(2) one or more positions on the antisense strand, complementary to nucleotides at one or more selected from 2^{nd}, 3^{rd}, 6^{th}, 8^{th} and 10^{th} to 13^{th} positions from the 5′ end of the sense strand,
whereby the double-stranded nucleic acid has increased in vivo stability.

Another aspect may provide a method for producing a double-stranded nucleic acid molecule or a radial nucleic add molecule (e.g., a nucleic acid molecule with increased in vivo stability and/or increased gene silencing activity), the method including:
(1) a step of synthesizing a polynucleotide strand including a region (region n-S) having a sequence identical to that of an n-th target gene and a region (region m-AS) having a sequence complementary to that of an m-th target gene, wherein the region n-S and the region m-AS each bear a chemically modified nucleotide at one or more of the following positions:
a) one or more positions in the region n-S, selected from the group consisting of 1^{st}, 4^{th}, 5^{th}, 7^{th} and 14^{th} positions from the 5′ end of the region n-S; and
b) one or more positions in the region m-AS, corresponding to one or more selected from the group consisting of 2^{nd}, 3^{rd}, 6^{th}, 8^{th} and 10^{th} to 13^{th} positions from the 5′ end of another polynucleotide strand complementary to the region m-AS (a separate polynucleotide strand including a region having the same sequence as the m-th target gene), and the n-th target gene and the m-th target gene are identical or are not identical to each other;
(2) a step of repeating the step of synthesizing the polynudeotide (step (1)) K times to synthesize K polynucleotide strands;
(3) a step of hybridizing the K polynucleotide strands synthesized in step (2);
(3) wherein the nucleic acid molecule indudes K radially extending arms,
K being an integer of 2≤K≤4,
each arm indudes double-stranded nucleic acid molecule,
the double-stranded nucleic acid molecules included in the individual arms are identical to or different from each other in terms of nucleotide sequence,
the region n-S may play the same role as the sense strand included in the double-stranded nudeic acid according to an embodiment and the region m-AS may play a role identical or similar to that of the antisense strand included in the double-stranded nucleic acid according to an embodiment.

In step (3), a region (region 1-1) containing the same sequence as the first target gene and a region (region 1-2) containing a complementary sequence to the first target gene are complementarily coupled with each other,
wherein region 1-1 and region 1-2 are included in respective separate polynucleotide strands.

The hybridization in step (3) is as described in the foregoing.

### Advantageous Effects

A double-stranded nucleic acid molecule or radial nudeic acid molecule containing a chemically modified nucleotide at a specific position may exhibit increased gene silencing activity against a target gene and increased in vivo stability.

### DESCRIPTION OF DRAWINGS

FIG. 1 is a schematic diagram of a double-stranded nucleic acid molecule bearing nucleotides chemically modified in a P(+1)P site-specific manner according to an embodiment.

FIG. 2 is a schematic diagram of a double-stranded nucleic acid molecule bearing nucleotides chemically modified in a P(−1)P site-specific manner according to an embodiment.

In FIGS. 1 and 2, SS stands for a sense strand, AS for an antisense strand, portions in green indicate chemically modified nucleotides, and portions in gray indicate chemically unmodified nudeotides.

FIG. 3 shows graphs of gene silencing activity of double-stranded nucleic acid molecules according to positions of chemically modified nucleotides therein.

FIG. 4 is a schematic diagram of a chemically modified nucleotide-bearing, double-stranded nucleic add molecule constructed in Example 2.

FIGS. 5A and 5B show Dicer reaction rates (left plots) and gene silencing activity (right graphs) of double-stranded nucleic acid molecules bearing chemically modified nudeotides at the positions indicated in FIG. 3.

FIG. 6 shows schematic diagrams of double-stranded nucleic acid molecules bearing nucleotides chemically modified according to conventional methods.

FIG. 7 shows Dicer reaction rates (left plot) and gene silencing activity (right graph) of double-stranded nucleic acid molecules bearing chemically modified nucleotides at the positions indicated in FIG. 6.

FIG. 8 is a graph of gene silencing activity of a double-stranded nudeic acid in which a chemical modification is made to the nudeotide at the 9^{th} position from the 5′ end of the is sense strand.

FIG. 9 is a schematic view of various Dicer substrate RNA constructs to which chemical modifications can be made in a site-specific manner according to an embodiment.

In FIG. 9, portions marked in red boxes mean sites into which chemical modifications can be introduced at specific positions according to an embodiment.

FIG. 10 shows Dicer reaction rates (left plot) and gene silencing activity (right graph) of linear dsRNA to which a chemical modification is made in a P(+1)P site-specific manner.

FIG. 11 is a plot of serum stability of various Dicer substrate RNA constructs to which a chemical modification is made in a P(+1)P site-specific manner according to an embodiment.

FIG. 12 is a plot elucidating immune induction ability of various Dicer substrate RNA constructs to which a chemical modification is made in a P(+1)P site-specific manner according to an embodiment.

FIG. 13 is a graph of in vitro and in vivo gene silencing activity of linear dsRNA to which a chemical modification is made in a P(+1)P site-specific manner according to an embodiment.

FIG. 14 is a graph of in vitro gene silencing activity against the target (HRRT) of a flanking-end dsRNA to which a chemical modification is made in a P(+1)P site-specific manner according to an embodiment.

FIG. 15 is a graph of in vivo gene silencing activity against the target (FVII) of a Y-RNA construct to which a chemical modification is made in a P(+1)P site-specific manner according to an embodiment.

FIG. 16 is a graph of in vivo gene silencing activity against various targets (FVII and TTR) of a Y-RNA construct to which a chemical modification is made in a P(+1)P site-specific manner according to an embodiment.

FIGS. 17 and 18 show measurements of in vivo gene silencing effects on FVII and TTR, respectively, according to concentrations of Dicer substrate RNAs to which a chemical modification is made in a P(+1)P site-specific manner according to an embodiment.

FIG. 19 is a graph of comparison of gene silencing effects among positions of P(+1)P site-specific chemical modifications according to an embodiment.

FIGS. 20 to 24 show results according to P(−1)P site-specific modifications according to an embodiments.

FIG. 20 is a graph of in vitro gene silencing activity of linear dsRNA to which a chemical modification is made in a P(−1)P site-specific manner according to an embodiment.

FIG. 21 is a graph of in vitro and in vivo gene silencing activity against various targets of linear dsRNA to which a chemical modification is made in a P(−1)P site-specific manner according to an embodiment.

FIG. 22 is a graph of gene silencing activity against various targets (FVII [symbol] TTR) of Y-RNA (nick) constructs to which a chemical modification is made in a P(−1)P site-specific manner according to an embodiment.

FIGS. 23 and 24 show measurements of in vivo gene silencing effects on FVII and TTR, respectively, according to concentrations of Dicer substrate RNAs to which a chemical modification is made in a P(−1)P site-specific manner according to an embodiment.

FIG. 25 is graph of gene silencing effects on GFP of Dicer substrate RNA constructs 23 nt and 30 nt long according to an embodiment.

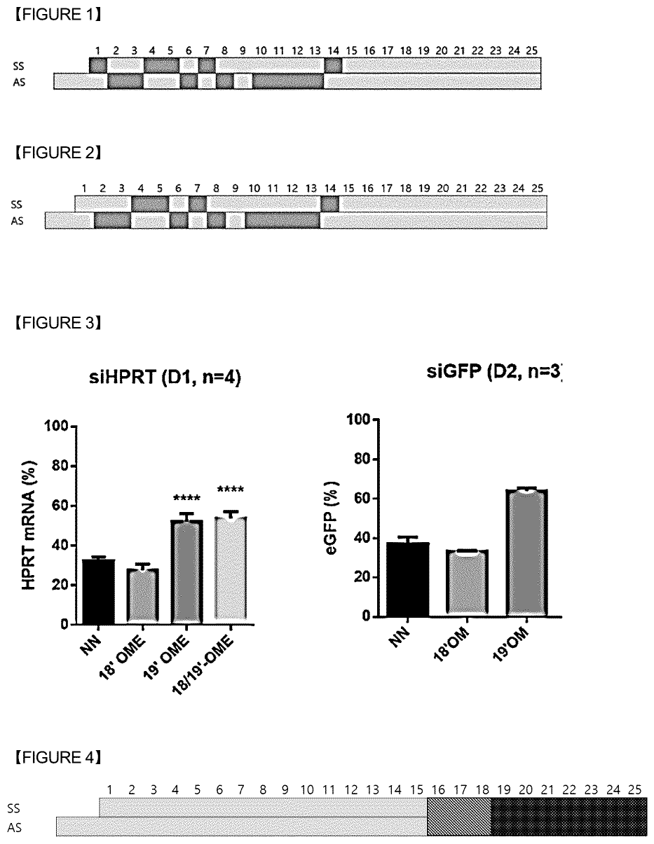

### MODE FOR INVENTION

A better understanding of the present disdosure may be obtained via the following examples which are set forth to illustrate, but are not to be construed as limiting the present disclosure.

### Example 1

### Gene Silencing Effect According to Chemical Modification Position in Antisense Strand

### Example 1-1

### Preparation of Dicer substrate RNA Bearing Chemically Modified Nucleotide in Antisense Strand

In the case of a double-stranded RNA (hereinafter referred to as dsRNA) having a length of as long as 25 to 35 bp, it is recognized by Dicer, an enzyme protein in the cytoplasm, and cut by the enzyme to a fragment about 20 to 25 bp in length, which shows a silencing effect on a target gene. Dicer is known to cut dsRNAs to a length of 20+2 or 21+2.

Among Dicer substrate RNAs capable of being prepared into dsRNA targeting HPRT and GFP, an antisense strand bearing a modified nucleotide (the 2′—OH group of the sugar ring moiety in the nucleotide was modified into a 2′-O-methyl group) capable of complementarily binding to the nucleotide located at the 7^{th} or 8^{th} position from the 5′ end of the sense strand was purchased from BIONEER. The nudeotides in the antisense strand, capable of complementarily binding to the nucleotides located at the 7^{th} and 8^{th} positions from the 5′ end of the sense strand, were located at the 19^{th} and 20^{th} positions from the 5′ ends of the antisense strand in the Dicer substrate RNA, respectively. Thereafter, the antisense strand in which the nucleotide at the position complementary to the nudeotide present at the 7^{th} position from the 5′ end of the sense strand was modified with a 2′-O-methyl group was named “19′ OME antisense strand”, and the antisense strand in which the nudeotide at the position complementary to the nucleotide at the 8^{th} position from the 5′ end of the sense strand is modified with a 2′-O-methyl group was named “18′ OME antisense strand”.

Among Dicer substrate RNAs capable of being prepared into dsRNA targeting HPRT and GFP, sense and antisense strands consisting of chemically unmodified nudeotides were also purchased from BIONEER. The nucleotide sequences of sense and antisense strands consisting of chemically modified and chemically unmodified nucleotides are listed in Table 2 (HPRT targeting sequence) and Table 3 (GFP targeting sequence) where the chemically modified nucleotides are indicated. In Tables 2 and 3, underlined nucleotides in bold had the -sugar ring moiety in which the 2′-OH group was modified into a 2′-O-methyl group.

TABLE 2

| | | SEQ ID |
|---|---|---|
| Strand | Sequence (5′→3′) | NO: |
| Sense strand | CCAGACUUUGUUGGAUUUGAAGUGC | 1 |
| (unmodified) | | |
| Unmodified | GCACUUCAAAUCCAACAAAGUCUGGCA | 2 |
| antisense | | |
| strand | | |
| 18′ OME | GCACUUCAAAUCCAACAAAGUCUGGCA | 3 |
| antisense | | |
| strand | | |
| 19′ OME | GCACUUCAAAUCCAACAAAGUCUGGCA | 4 |
| antisense | | |
| strand | | |
| 18/19′ | GCACUUCAAAUCCAACAAAGUCUGGCA | 5 |
| strand OME | | |
| antisense | | |

TABLE 3

| | | SEQ ID |
|---|---|---|
| Strand | Sequence (5′→3′) | NO: |
| Sense strand | GCAAGCUGACCCUGAAGUUAUCACC | 6 |
| (unmodified) | | |
| Unmodified | GGUGAUAACUUCAGGGUCAGCUUGCCA | 7 |
| antisense | | |
| strand | | |
| 18′ OME | GGUGAUAACUUCAGGGUCAGCUUGCCA | 8 |
| antisense | | |
| strand | | |
| 19′ OME | GGUGAUAACUUCAGGGUCAGCUUGCCA | 9 |
| antisense | | |
| strand | | |

To prepare Dicer substrate RNA, a sense strand and an antisense strand were mixed at an amount of 10 pmoles each in the following combination, and hybridized into a double-stranded strand by incubation starting at 95° C. for 3 minutes, with a subsequent temperature decrease from 95° C. to 4° C. at a rate of −1.0° C./s in a thermal cycler (Bio-Rad T100™):
(1) Dicer substrate dsRNA targeting HPRT: (a) sense strand/unmodified antisense strand; (b) sense strand/antisense strand having a modified nudeotide at the 18^{th} position from the 5′ end; (c) sense strand/antisense strand having a modified nudeotide at the 19^{th} position from the 5′ end; and (d) sense strand/antisense strand having modified nudeotides at the 18^{th} and 19^{th} positions from the 5′ end.
(2) Dicer substrate dsRNA targeting GFP: (a) sense strand/antisense strand; (b) sense strand/antisense strand having a modified nudeotide at the 18^{th} position from the 5′ end; and (c) sense strand/antisense strand having a modified nucleotide at the 19^{th} position from the 5′ end.

### Example 1-2

### RT-PCR Assay for Gene Silencing Effect In Vitro

In this Example, the HPRT gene silencing effect of the Dicer substrate RNA prepared in Example 1-1 was assayed in vitro through real-time polymerase chain reaction (real-time (RT)-PCR).

GFP-KB cells (KB cells designed to express GFP (green fluorescent protein)) were seeded at a density of 0.1×10⁶ cells/well into 96-well transparent culture plates containing RPMI medium (10% fetal bovine serum, 1% penicillin/streptomycin). After incubation at 37° C. for 14 hours in a 5% CO₂ condition, four Dicer substrate dsRNA samples targeting HPRT, prepared in Example 1-1, were each mixed with Lipofectamine® RNAiMAX (Invitrogen) so that the RNA amount in the final composition (500 μl) was 0.1 pmole, and then left at room temperature for 5 min. Subsequently, 450 μl of RPMI medium was added to form a total of 500 μl in each well to which the samples were each applied in an amount of 100 μl (N=4) to transfect the Dicer substrate RNA sample into GFP-KB cells.

Thereafter, the cells were incubated for 24 hours and RNA was extracted from dsRNA sample-transfected GFP-KB cells, using CellAmp™ Direct RNA Prep Kit for RT-PCR (TAKARA). The extracted RNA was mixed with One Step TB Green® PrimeScript™ RT-PCR Kit II (Takara) including forward and reverse primers to prepare a final volume of 20 μl. The primer sequences used are listed in Table 4, below. In this regard, the samples for RT-PCR induded a sample for the HPRT gene to be evaluated for the silencing effect by the Dicer substrate RNA prepared in Example 1-1 and a sample for the GAPDH gene to be used as an internal control for result correction. PCR amplification was performed using the RT- CFX96 Touch Real-Time PCR Detection System (BioRad). Amplification was initiated at 42° C. for 5 minutes and then at 95° C. for 10 seconds and proceeded with 40 cycles of 95° C. for 5 seconds at 60° C. for 30 seconds, and 72° C. for 30 seconds. Ct stands for a threshold cycle, ΔCt for a value obtained by subtracting the average Ct value of the internal control GAPDH mRNA from that of HPRT mRNA, and ΔACt for a difference in ΔCt between an experimental group treated with sample dsRNA and a negative control treated separately with RNA. A change in HPRT expression level was calculated using the delta-delta Ct calculation method, and the results are depicted in the left graph of FIG. 3. The significance of the analysis was verified through correlation between the modified Dicer substrate RNA-treated group and the unmodified Dicer substrate RNA-treated group by Ordinary one-way ANOVA analysis (Graphpad Prism 6).

TABLE 4

| Name | Sequence (5′→3′) | SEQ ID NO: |
|---|---|---|
| HRRT_Forward | GACTTTGCTTTCCTTGGTCAG | 10 |
| HRRT_Reverse | GGCTTATATCCAACACTTCGTGGG | 11 |
| GAPDH_Foward | GGATTTGGTCGTATTGGG | 12 |
| GAPDH_Reverse | GGAAGATGGTGATGGGATT | 13 |

As shown in the left graph of FIG. 3, the Dicer substrate dsRNA-treated group with a modified nucleotide at the 18^{th} position from the 5′ end of the antisense (18′OME) was not 20 lower in gene silencing effect than the unmodified Dicer substrate dsRNA sample-treated group (NN). However, the Dicer substrate dsRNA-treated group with a modified nucleotide at the 19^{th} position from the 5′ end of the antisense (19′OME) significantly increased HPRT mRNA expression, compared to NN. This was true of the Dicer substrate dsRNA-treated group with modified nucleotides at the 18^{th} and 19^{th} positions from the 5′ end of the antisense (18/19′OME). From the data, it was understood that the modification of the 19^{th} nucleotide from the 5′ end of the antisense significantly reduced the gene silencing effect of Dicer substrate RNA.

### Examples 1-3

### FACS Assay for Gene Silencing Effect In Vitro

In this Example, the GFP gene silencing effect of the Dicer substrate dsRNA prepared in Example 1-1 was assayed in vitro through fluorescence activated cell sorting (FACS).

GFP-KB cells were seeded at a density of 1.0×10⁶ cells/well into 12-well culture plates containing RPMI medium. After incubation at 37° C. for 24 hours in a 5% CO₂ condition, the three Dicer substrate dsRNA samples targeting GFP, prepared in Example 1-1, were each mixed at an amount of 0.2 pmoles with 3 μl of Lipofectamine® RNAiMAX (Invitrogen) and then, left at room temperature for 5 minutes. The samples were each applied to at a final concentration of 0.2 nM to the GFP-KB cells seeded into the wells, followed by incubation for 48 hours. Thereafter, the GFP expression level of GFP-KB cells was measured using a Novocyte 2060R Flow cytometer (ACEA Biosciences), and the GFP expression level was analyzed with ACEA NovoExpress software. The results are depicted in the right graph of FIG. 3.

As shown in the right graph of FIG. 3, the Dicer substrate dsRNA-treated group with a modified nucleotide at the 18^{th} position from the 5′ end of the antisense (18′OME) was not lower in gene silencing effect than the unmodified Dicer substrate dsRNA sample-treated group (NN). However, the Dicer substrate dsRNA-treated group with a modified nucleotide at the 19^{th} position from the 5′ end of the antisense (19′OME) significantly increased HPRT mRNA expression, compared to NN. From the data, it was understood that the modification of the 19^{th} nucleotide from the 5′ end of the antisense significantly reduced the gene silencing effect of Dicer substrate RNA. Consistent with that of Example 1-2, the data is interpreted to have no influences from gene sequences.

For use in subsequent experiments, the Dicer substrate RNAs were thus modified, based on the results of Examples 1-2 and 1-3, as follows: the nucleotide at the 19^{th} position from the 5′ end of the antisense in Dicer substrate RNA (=the nucleotide complementary to the nudeotide at the 7^{th} position from the 5′ end of the sense) was not chemically modified while the nucleotide at the 18^{th} position from the 5′ end of the antisense in Dicer substrate RNA (=the nucleotide complementary to the nudeotide at the 8^{th} position from the 5′ end of the sense) was chemically modified.

### Example 2

### Effect According to Position of Chemical Modification

In this example, samples in which modification was made to the nucleotides present within the 15^{th} position, at 16^{th} to 18^{th} positions, and at 19^{th} to 21^{st} positions from the 5′ end of to the sense strand in the Dicer substrate RNA were measured for Dicer reaction rate and in vitro gene silencing effect.

### Example 2-1

### Preparation of Chemically Modified Dicer Substrate RNA

Dicer substrate RNAs capable of being prepared into cleaved dsRNA targeting HPRT is as divided into three groups: (1) unmodified Dicer substrate RNA (hereinafter, control); (2) Dicer substrate RNA with a modification made to the nucleotides present at 21^{st} to 23^{rd} positions from the 5′ end of the sense strand and to the nucleotides on the antisense strand, present at positions corresponding to 19^{th} to 21^{st} positions from the 5′ end of the sense strand and complementarily binding to the nucleotides the sense strand (hereinafter, 19-21 bp modification group); (3) Dicer substrate RNA with a modification made to the nudeotides present within the 15^{th} position from the sense strand and to the nucleotides present at corresponding positions in the antisense strand and complementarily binding to the nucleotides of the sense strand (hereinafter, ˜15 bp modification group), and (4) Dicer substrate RNA with a modification made to the nudeotides present 16^{th} to 18^{th} positions from the 5′ end of the sense strand and the nucleotides present at corresponding positions in the antisense strand and complementarily binding to the nucleotides of the sense strand plus the modification of (3) (hereinafter, ˜15bp +16-19 bp modification group). The specific modification positions in each group are given in Table 5, below. Nucleotides indicated in bold and underlined in Table 5 mean that the 2′-OH group of the sugar was modified into a 2′-O-methyl group.

TABLE 5

| | | | SEQ |
|---|---|---|---|
| Strand | | Sequence (5′→3′) | ID NO: |
| Unmodified | sense strand | CCAGACUUUGUUGGAUUUGAAGUGC | 1 |
| | antisense strand | GCACUUCAAAUCCAACAAAGUCUGGCA | 2 |
| 19-21 bp | sense strand | CCAGACUUUGUUGGAUUUGAAGUGC | 14 |
| Modified | antisense strand | GCACUUCAAAUCCAACAAAGUCUGGCA | 15 |
| ~15 bp | sense strand | CCAGACUUUGUUGGAUUUGAAGUGC | 16 |
| Modified | antisense strand | GCACUUCAAAUCCAACAAAGUCUGGCA | 17 |
| ~15 bp + | sense strand | CCAGACUUUGUUGGAUUUGAAGUGC | 18 |
| 16-19 bp | antisense strand | GCACUUCAAAUCCAACAAAGUCUGGCA | 19 |
| Modified | | | |

The sense strands and antisense strands composed of the nucleotide sequences and having modified nucleotides (2-O-methyl group) as listed in Table 5 were purchased from BIONEER. The sense and antisense strands were each mixed at an amount of 10 pmoles and hybridized into double strands by incubation starting at 95° C. for 3 minutes, with a subsequent temperature decrease from 95° C. to 4° C. at a rate of −1.0° C./s in a thermal cycler (Bio-Rad T100™).

### Example 2-2

### Dicer Kinetic Analysis

The four Dicer substrate RNA samples, prepared in Example 2-1, were each mixed is in an amount of 10 pmoles with 5 pmoles of recombinant human Dicer, 1 μl of a Dicer reaction buffer (300mM Tris-HCl (pH 6.8), 500 mM NaCl), 2 μl of DEPC-treated deionized water (DVV), and 2 μl of 25 mM MgCl₂. The resulting mixture was incubated at 37° C. for 6 hours in a thermal cycler to allow the Dicer to cleave the Dicer substrate RNA.

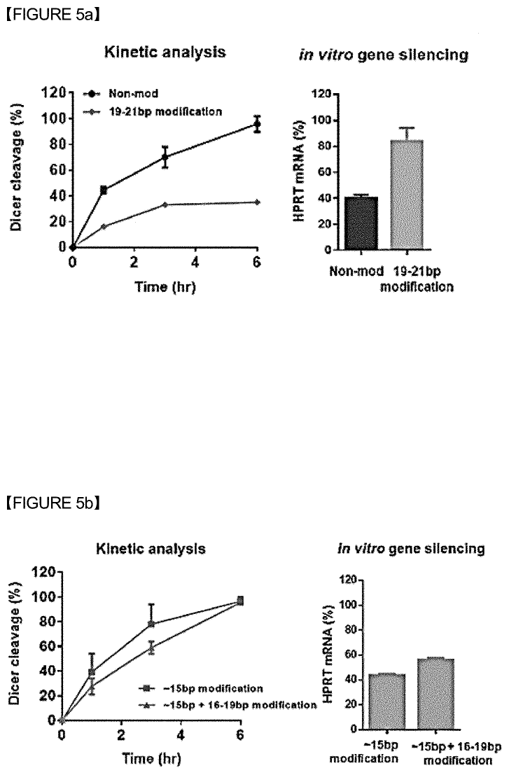

After 0, 1, 3, and 6 hours of the incubation, 1 μl of each sample was mixed with 8 μl of 1× Tris-borate-EDTA(TBE) buffer and 2 μl of 6× gel loading dye (Biolabs) to prepare an electrophoresis sample. The electrophoresis samples were loaded in an amount of 1 1 μl to each well in 15-well 10% PAGE (polyacrylamide gel electrophoresis) gel. After gel running for 40 minutes at 200 V, the gel was stained with Gel red (Biotium) and a gel image was obtained using Gel DocTM EZ (Bio-Rad). The Dicer cleavage (%) was calculated from the change in the thickness of the Dicer substrate band with time, based on 0 hours of the reaction and the results are depicted in the left plots of FIGS. 5A and 5B.

As shown in the left plots of FIGS. 5A and 5B, the 19-21 bp modification group had a reduced Dicer processing speed for the Dicer substrate RNA, compared to the chemically unmodified control, and the ˜15 bp+16-19 bp modification group had a reduced Dicer processing speed for the Dicer substrate RNA, compared to the ˜15 bp modification group.

### Example 2-3

### RT-PCR Assay for Gene Silencing Effect In Vitro

In this Example, the four Dicer substrate RNAs prepared in Example 2-1 were applied is to GFP-KB cells in the same manner as in Example 1-2, and the silencing effect on HRRT, a target gene, in vitro was measured through RT-PCR. The results are shown in the right graphs of FIGS. 5A and 5B. The significance was verified through correlation among the modified Dicer substrate RNA-treated groups and the unmodified Dicer substrate RNA-treated group by Ordinary one-way ANOVA analysis (Graphpad Prism 6).

As shown in the right graphs of FIGS. 5A and 5B, a significant decrease of gene silencing effect was detected in the 19-21 bp modification group, compared to the chemically unmodified control and in the ˜15 bp+16-19 bp modification group, compared to the ˜15 bp modification group.

Therefore, in the subsequent Examples, experiments were conducted with the Dicer substrate RNA in which the nucleotides within the 15^{th} position from the 5′ end of the sense strand and the nucleotides complementary thereto on the antisense strand were chemically modified.

EXAMPLE 3

### Effect of Chemical Modification Made to Nucleotides Within 15^{th} Position from 5′ End of Sense Strand

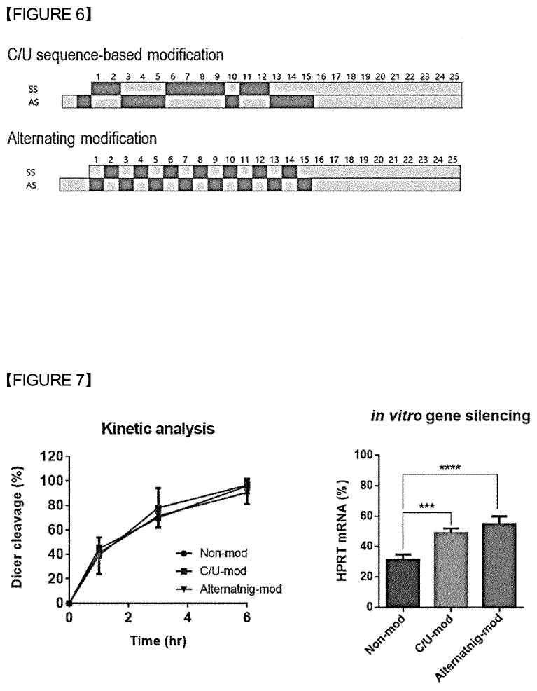

In order to examine the effect of chemical modification made to the nucleotides present within the 15^{th} position from the 5′ end of the sense strand and the nucleotides at the corresponding positions on the antisense strand complementary thereto, the well-known conventional chemical modification methods (1) C/U sequence-based modification to chemically modify C and U nucleotides, and (2) alternating modification to chemically modify nucleotides alternately in the sense and antisense strands were used to prepare HPRT-targeting Dicer substrate dsRNAs, and their Dicer cleavage rates and gene silencing effects were measured in vitro. The chemical modification positions of the Dicer substrate dsRNAs according to the C/U sequence-based modification method and the alternating modification method are shown in FIG. 6. Nucleotides at positions marked in red in FIG. 6 represent chemically modified nudeotides.

### Example 3-1

### Preparation of Chemically Modified Dicer Substrate RNA

Based on the results of Example 2 above, experiments were conducted with Dicer substrate dsRNA in which nudeotides within the 15^{th} position from the 5′ end of the sense strand and nucleotides at the corresponding positions on the antisense strand complementary thereto were chemically modified.

HPRT-targeting Dicer substrate dsRNAs in which nucleotides of the sense strand and antisense strand were modified at the positions indicated in Table 6 by (1) C/U sequence-based modification and (2) alternating modification were synthesized by and purchased from BIONEER. In Table 6, underlined nudeotides in bold had the sugar ring moiety in which the 2′-OH group was modified into a 2′-O-methyl group.

TABLE 6

| | | | SEQ |
|---|---|---|---|
| | | | ID |
| Strand | | Sequence (5′→3′) | NO: |
| Unmodified | sense strand | CCAGACUUUGUUGGAUUUGAAGUGC | 1 |
| (Non-mod) | antisense strand | GCACUUCAAAUCCAACAAAGUCUGGCA | 2 |
| C/U sequence- | sense strand | CCAGACUUUGUUGGAUUUGAAGUGC | 20 |
| based | antisense strand | GCACUUCAAAUCCAACAAAGUCUGGCA | 21 |
| modification | | | |
| (C/U-mod) | | | |
| Altemating | sense strand | CCAGACUUUGUUGGAUUUGAAGUGC | 22 |
| modification | antisense strand | GCACUUCAAAUCCAACAAAGUCUGGCA | 23 |
| (Alternating-mod) | | | |

The sense and antisense strands were each mixed at an amount of 100 pmoles and hybridized into double strands by incubation starting at 95° C. for 3 minutes, with a subsequent temperature decrease from 95° C. to 4° C. at a rate of −1.0° C./s in a thermal cycler (Bio-Rad T100™).

### Example 3-2

### Dicer Kinetic Analysis

The Dicer cleavage (%) of the three Dicer substrate RNA samples prepared in Example 3-1 was calculated in the same manner as in Example 2-2, and the results are depicted in the left plot of FIG. 7.

As shown in FIG. 7, the Dicer processing rate was not decreased for the Dicer substrate RNA in which the nucleotides within the 15^{th} position from the 5′ end of the sense strand and the nucleotides at the corresponding positions on the antisense strand complementary thereto were chemically modified, compared to unmodified Dicer substrate RNA, irrespective of the modification method (C/U sequence-based modification method or alternating modification method).

### Example 3-3

### RT-PCR Assay for Gene Silencing Effect In Vitro

In this Example, the three Dicer substrate RNAs prepared in Example 3-1 were applied to GFP-KB cells in the same manner as in Example 1-2, and the silencing effect on HRRT, a target gene, in vitro was measured through RT-PCR. The results are shown in the right plot of FIG. 7. The significance was verified through correlation among the modified Dicer substrate RNA-treated groups and the unmodified Dicer substrate RNA-treated group by Ordinary one-way ANOVA analysis (Graphpad Prism 6).

As shown in FIG. 7, a significant decrease of gene silencing effect was detected in vitro in all the Dicer substrate RNAs in which the nudeotides with the 15^{th} positions from the end of the sense strand and the nucleotides at corresponding positions on the antisense strand complementary thereto were chemically modified by C/U sequence-based modification or alternating modification methods.

### Example 4

### Effect of Chemical Modification Made to Nucleotide at 9^{th} Position from 5′ End of Sense Strand

A chemical modification method was designed to increase a gene silencing effect in vivo without reducing the Dicer processing rate and the in vitro gene silencing effect, although the nucleotides within the 15^{th} from the 5′ end of the sense strand and the nudeotides at corresponding positions on the antisense strand complementary thereto were chemically modified.

### Example 4-1

### Preparation of Dicer Substrate RNA with Site-Specific Modification

The sense and antisense strands consisting of the sequences with chemically modified nucleotides at the positions shown in Table 7, below were synthesized in and purchased from BIONEER. Dicer substrate dsRNAs were prepared such that the nucleotides at 1^{st}, 4^{th}, 5^{th}, 7^{th} and 14^{th} positions from the 5′ end of the sense strand and the nucleotides on the antisense strand at positions corresponding to 2^{nd}, 3^{rd}, 6^{th}, 8^{th}, and 10^{th}-13^{th} positions from the 5′ end of the sense strand complementary thereto were chemically modified in a site-specific manner. This modification method was named P(+1)P site-specific chemical modification method in subsequent Examples. Modification of the nucleotide at the 9^{th} position from the 5′ end of the sense strand in Dicer substrate RNAs by the site-specific chemical modification method was examined for influence on gene silencing and Dicer deavage processing. In Table 7, below, underlined nucleotides in bold had the sugar ring moiety in which the 2′-OH group was modified into a 2′-O-methyl group.

TABLE 7

| Strand | | Sequence (5′→3′) | SEQ ID NO: |
|---|---|---|---|
| Unmodified | Sense strand | GCAAGCUGACCCUGAAGUUAUC | 6 |
| | | ACC | |
| | Antisense strand | GGUGAUAACUUCAGGGUCAGCU | 7 |
| | | UGCCA | |
| Site-specific | Sense strand | GCAAGCUGACCCUGAAGUUAUC | 24 |
| modification (9′- | | ACC | |
| OMe inclusive) | Antisense strand | GGUGAUAACUUCAGGGUCAGCU | 25 |
| | | UGCCA | |
| Site-specific | Sense strand | GCAAGCUGACCCUGAAGUUAUC | 26 |
| modification (9′- | | ACC | |
| OMe exclusive) | Antisense strand | GGUGAUAACUUCAGGGUCAGCU | 25 |
| | | UGCCA | |

The sense strands and antisense strands purchased from BIONEER were each mixed at an amount of 100 pmoles and hybridized into double strands by incubation starting at 95° C. for 3 minutes, with a subsequent temperature decrease from 95° C. to 4° C. at a rate to of −1.0° C./s in a thermal cycler (Bio-Rad T100™), to prepare (1) a chemically unmodified control (non-mod), (2) a Dicer substrate dsRNA with a P(+1)P site-specific modification and an additional 9′-OMe chemical modification (PP wt 9 nt mod), and (3) Dicer substrate dsRNA with a P(+1)P site-specific modification and no 9′-OMe chemical modification (PP wo 9 nt mod).

### Example 4-2

### FACS Assay for Gene Silencing Effect

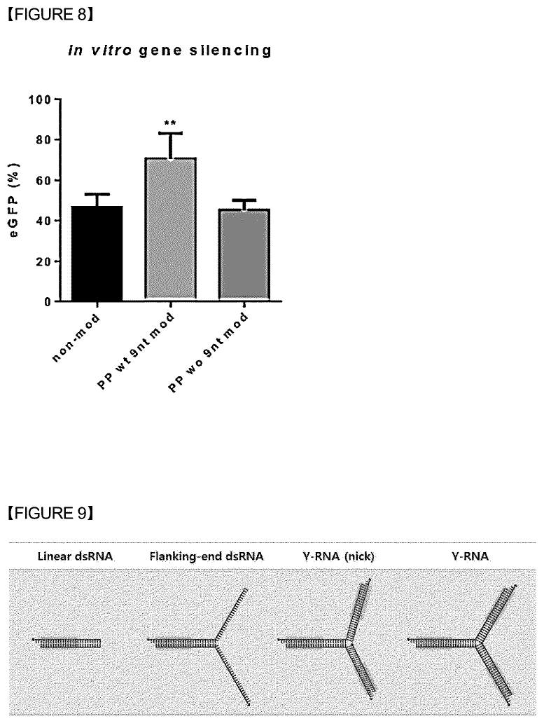

The three Dicer substrate dsRNA samples prepared in Example 4-1 were applied to GFP-KB cells, and the expression level of the target gene GFP was measured in vitro in the same manner as in Example 1-3 through FACS. Thus, the results are depicted in FIG. 8.

As shown in FIG. 8, the Dicer substrate dsRNA with a P(+1)P site-specific modification and an additional 9′-OMe chemical modification exhibited a lower gene silencing effect for GFP than the Dicer substrate RNA with no chemical modifications. On the other hand, the Dicer substrate dsRNA with a P(+1)P site-specific modification and no 9′-OMe chemical modifications did not decrease in gene silencing effect for GFP, compared to the chemically unmodified Dicer substrate RNA.

### Example 5

### Dicer Substrate RNA Chemically Modified in P(+1)P Site-Specific Manner

Based on the results of Examples 3 and 4, Dicer substrate dsRNA composed of a sense strand and an antisense strand was prepared in a site-specific manner such that the nucleotides at 1^{st}, 4^{th}, 5^{th}, 7^{th} and 14^{th} positions from the 5′ end of the sense strand and the nucleotides on the antisense strand at positions corresponding to the 2^{nd} 3^{nd} 6^{th}, 8^{th}, and 10^{th} to 13^{th} positions from 5′ end of the sense strand were chemically modified in a site-specific manner. This chemical modification was named P(+1)P site-specific chemical modification method.

Dicer substrate dsRNAs were prepared by a P(+1)P site-specific chemical modification method and measured for Dicer reaction rate and gene silencing effect in vitro. The structure of the Dicer substrate dsRNA chemically modified by a site-specific method according to an embodiment is exemplified as shown in FIG. 9.

### Example 5-1

### Preparation of Dicer Substrate RNA Chemically Modified In Site-Specific Manner

In order to prepare linear Dicer substrate RNA capable of being produced into cleaved dsRNA (dsRNA product by Dicer cleavage, cleaved product), sense and antisense strands having the sequences shown in Table 8, below, with chemically modified nudeotides at the indicated positions, were synthesized in and purchased from BIONEER. In Table 8, below, underlined nucleotides in bold had the sugar ring moiety in which the 2′-OH group was modified into a 2′-O-methyl group.

TABLE 8

| | | | SEQ |
|---|---|---|---|
| | | | ID |
| Strand | | Sequence (5′→3′) | NO: |
| Chemically | Sense strand | CCAGACUUUGUUGGAUUUGAAGUGC | 1 |
| unmodified (NN) | Antisense strand | GCACUUCAAAUCCAACAAAGUCUGGCA | 2 |
| P(+1)P site-specific | Sense strand | CCAGACUUUGUUGGAUUUGAAGUGC | 27 |
| modification | Antisense strand | GCACUUCAAAUCCAACAAAGUCUGGCA | 28 |
| C/U sequence- | Sense strand | CCAGACUUUGUUGGAUUUGAAGUGC | 20 |
| based modification | Antisense strand | GCACUUCAAAUCCAACAAAGUCUGGCA | 21 |
| Alternating | Sense strand | CCAGACUUUGUUGGAUUUGAAGUGC | 22 |
| modification | Antisense strand | GCACUUCAAAUCCAACAAAGUCUGGCA | 23 |

The sense strands and antisense strands purchased from BIONEER were each mixed in an amount of 100 pmoles and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare the following Dicer substrate dsRNAs capable of being prepared into cleaved dsRNA targeting HRRT: (1) a chemically unmodified control (NN), (2) Dicer substrate linear dsRNA with a P(+1)P site-specific modification (P(+1)P), (3) Dicer substrate linear dsRNA with a C/U sequence-based chemical modification (CU-mod), and (4) Dicer substrate linear dsRNA with an alternating chemical modification (Alt-mod).

### Example 5-2

### In Vitro Dicer Kinetic Analysis and Gene Silencing Effect According to P(+1)P Site-Specific Chemical Modification

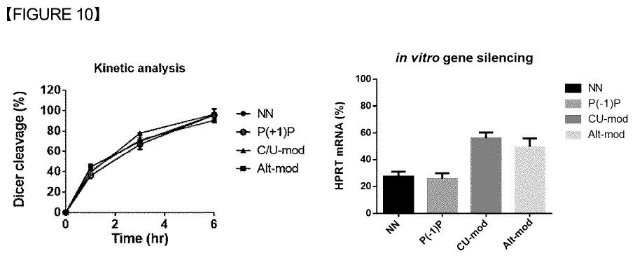

Dicer cleavage rates (%) of the four Dicer substrate RNA samples prepared in Example 5-1 were calculated in the same manner as in Example 2-2, and the results are depicted in the left plot of FIG. 10. The silencing effect on the target gene HRRT was measured in vitro through RT-PCR, and the results are depicted in the right graph of FIG. 10.

As shown in the left plot of FIG. 10, none of the chemically unmodified Dicer substrate RNA, the Dicer substrate RNA according to the alternating modification method, and the Dicer substrate modified in the site-specific manner of Example 5-1 showed a decrease in Dicer processing rate. As shown in the right graph of FIG. 10, the Dicer substrate RNA chemically modified in a site-specific manner in vitro did decrease in gene silencing effect, compared to unmodified Dicer substrate RNA. In contrast, the Dicer substrate RNAs chemically modified by the alternating modification method and the C/U sequence-based modification method exhibited lower gene silencing effects, compared to the unmodified Dicer substrate RNA.

### Example 6

### Assay for Serum Stability of Dicer Substrate RNA Chemically Modified in P(+1)P Site-Specific Manner

Various Dicer substrate RNA structures (linear dsRNA, Y-RNA structure, and Y-RNA structure with a nick therein) chemically modified in a P(+1)P site-specific manner were assayed for serum stability.

### Example 6-1

### Preparation of Chemically Modified Dicer Substrate RNA

In order to prepare various Dicer substrate RNAs capable of being produced into deaved dsRNA, sense and antisense strands having the sequences shown in Table 9, below, with chemically modified nudeotides at the indicated positions, were synthesized in and purchased from BIONEER. In Table 9, below, underlined nucleotides in bold had the sugar ring moiety in which the 2′-OH group was modified into a 2′-O-methyl group.

TABLE 9

| | | | SEQ |
|---|---|---|---|
| | | | ID |
| Strand | | Sequence (5′→3′) | NO: |
| SiRNA | sense strand | CCAGACUUUGUUGGAUUUGTT | 29 |
| (19 + 2, | antisense | CAAAUCCAACAAAGUCUGGTT | 30 |
| chemically | strand | | |
| unmodified) | | | |
| Dicer | sense strand | CCAGACUUUGUUGGAUUUGAAGUGC | 1 |
| substrate | antisense | GCACUUCAAAUCCAACAAAGUCUGGCA | 2 |
| linear RNA | strand | | |
| chemically | | | |
| unmodified | | | |
| Linear | sense strand | CCAGACUUUGUUGGAUUUGAAGUGC | 27 |
| RNA in | antisense | GCACUUCAAAUCCAACAAAGUCUGGCA | 28 |
| P(+1)P | strand | | |
| site- | | | |
| specific | | | |
| modification | | | |
| Y-RNA | Strand 1 | CCAGACUUUGUUGGAUUUGAAGUGCGGUGAUGUA | 31 |
| (nick) in | (HPRT/FVII) | AG | |
| P(+1)P | | ACUUGAGAUGAUCCCA | |
| site- | Strand 2 | GGAUCAUCUCAAGUCUUACAUCACCCGUCUAUUAU | 32 |
| specific | (FVII/mTTR) | A | |
| modification | | GAGCAAGAACACUGCA | |
| | Strand 3 | CAGUGUUCUUGCUCUAUAAUAGACG | 34 |
| | (mTTR sense) | | |
| | Strand 4 | GCACUUCAAAUCCAACAAAGUCUGGCA | 28 |
| | (HPRT, | | |
| | antisense) | | |
| Y-RNA in | Strand 1 | CCAGACUUUGUUGGAUUUGAAGUGCGGUGAUGUA | 31 |
| P(+1)P | (HPRT/FVII) | AG | |
| site- | | ACUUGAGAUGAUCCCA | |
| specific | Strand 2 | GGAUCAUCUCAAGUCUUACAUCACCCGUCUAUUAU | 32 |
| modification | (FVII/mTTR) | A | |
| | | GAGCAAGAACACUGCA | |
| | Strand | CAGUGUUCUUGCUCUAUAAUAGACGGCACUUCAAA | 33 |
| | 3(mTTR/HPRT) | U | |
| | | CCAACAAAGUCUGGCA | |

The sense strands and antisense strands were each mixed at an amount of 100 pmoles and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare siRNA and linear dsRNA.

The Y-RNA construct or the Y-RNA construct with a nick therein were prepared by mixing 100 pmoles of each of three or four strands and hybridizing the strands into double strands through incubation starting at 95° C. for 3 minutes, with a subsequent temperature decrease from 95° C. to 4° C. at a rate of −1.0° C./s in a thermal cycler (Bio-Rad T100™). The Y-RNA structure is a radial nucleic acid structure having linear dsRNAs as arms that can be prepared into cleaved dsRNAs targeting HRRT, FVII, and mTTR, respectively. The Y-RNA structure with a nick is a radial nucleic acid structure having a nick at the center thereof and linear dsRNAs as arms that can be prepared into cleaved dsRNAs targeting HRRT, FVII, and mTTR, respectively. Structures of linear dsRNA, Y-RNA, and Y-RNA with a nick are is depicted in FIG. 9.

### Example 6-2

### Serum Stability Test

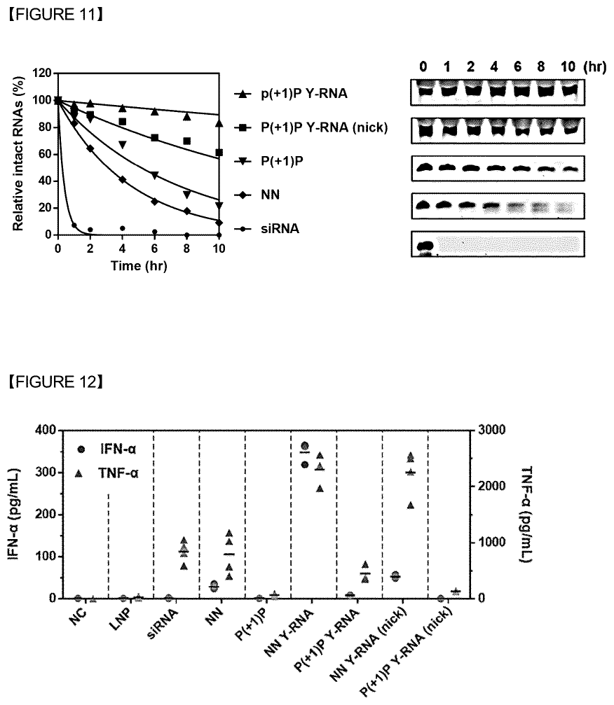

Blood was collected from untreated C57bl/6NCrSlc mice (female, 18-22g) and then centrifuged at 4° C. and 2000g for 15 minutes to separate serum. Eleven pmoles of each of the RNA samples prepared in Example 6-1 was added to MgCl₂ and the mouse serum to form a final volume of 44 μl. In this regard, the RNA sample and MgCl₂ had final concentrations of 0.25 μM and 5 mM, respectively. The stability of each RNA sample in the serum was compared during incubation at 37° C. for 10 hours. At 0, 2, 4, 6, 8, and 10 hours of the reaction, 4 μl of each sample was collected and mixed with 1 μl of 0.5 M EDTA, 6 μl of 1× TBE buffer, and 2.2 μl of 6× gel loading dye (Biolabs) to stop the reaction. The reaction mixture was loaded in an amount of 13.2 μl per well into 10% or 15% PAGE gel and run at 150V for 30 minutes or at 200V for 40 minutes. The gel was stained with Gel red (Biotium). A gel image was obtained using Gel DocTM EZ (Bio-Rad) and is depicted in FIG. 11 (right).

The serum stability was calculated from the change in the oligo band thickness with time, based on 0 hours of the reaction and the results are depicted in the left plot of FIG. 11.

As shown in FIG. 11, the Dicer substrate linear dsRNA (NN) was superior in serum stability to siRNA, and when the Dicer substrate dsRNA was chemically modified in a site-specific manner according to an embodiment, the linear construct (P(+1) P), Y-RNA construct (P(+1)P Y-RNA), and Y-RNA construct with nick (P(+1)P Y-RNA (nick)) were all observed to exhibit higher serum stability than chemically unmodified Dicer substrate (NN).

### Example 7

### Assay for Immune Induction of Dicer Substrate RNA Chemically Modified in P(+1)P Site-Specific Manner

### Example 7-1

### Preparation of Chemically Modified Dicer Substrate RNA

In order to prepare various Dicer substrate RNAs capable of being produced into deaved dsRNA, sense and antisense strands having the sequences shown in Table 10, below, with chemically modified nudeotides at the indicated positions, were synthesized in and purchased from BIONEER. In Table 10, below, underlined nucleotides in bold had the sugar ring moiety in which the 2′-OH group was modified into a 2′-O-methyl group.

TABLE 10

| | | | | SEQ ID |
|---|---|---|---|---|
| Strand | | | Sequence (5′→3′) | NO: |
| SiRNA (19+2, | HPRT | Sense strand | CCAGACUUUGUUGGAUUUGTT | 29 |
| chemically | | Antisense strand | CAAAUCCAACAAAGUCUGGTT | 30 |
| unmodified) | mTTR | Sense strand | CAGUGUUCUUGCUCUAUAATT | 35 |
| | | Antisense strand | UUAUAGAGCAAGAACACUGTT | 36 |
| | FVII | Sense strand | GGAUCAUCUCAAGUCUUACTT | 37 |
| | | Antisense strand | GUAAGACUUGAGAUGAUCCTT | 38 |
| Dicer | HPRT | Sense strand | CCAGACUUUGUUGGAUUUGAAGU | 1 |
| substrate | | | GC | |
| linear RNA | | Antisense strand | GCACUUCAAAUCCAACAAAGUCUG | 2 |
| chemically | | | GCA | |
| unmodified | mTTR | Sense strand | CAGUGUUCUUGCUCUAUAAUAGA | 39 |
| (NN) | | | CG | |
| | | Antisense strand | CGUCUAUUAUAGAGCAAGAACACU | 40 |
| | | | GCA | |
| | FVII | Sense strand | GGAUCAUCUCAAGUCUUACAUCAC | 41 |
| | | | C | |
| | | Antisense strand | GGUGAUGUAAGACUUGAGAUGAU | 42 |
| | | | CCCA | |
| Dicer | HPRT | Sense strand | CCAGACUUUGUUGGAUUUGAAGU | 27 |
| substrate | | | GC | |
| linear RNA in | | Antisense strand | GCACUUCAAAUCCAACAAAGUCU | 28 |
| P(+1)P site- | | | GGCA | |
| specific | mTTR | Sense strand | CAGUGUUCUUGCUCUAUAAUAGA | 34 |
| modification | | | CG | |
| | | Antisense strand | CGUCUAUUAUAGAGCAAGAACAC | 43 |
| | | | UGCA | |
| | FVII | Sense strand | GGAUCAUCUCAAGUCUUACAUCAC | 44 |
| | | | C | |
| | | Antisense strand | GGUGAUGUAAGACUUGAGAUGAU | 45 |
| | | | CCCA | |

| | | | |
|---|---|---|---|
| Y-RNA | Strand 1 | CCAGACUUUGUUGGAUUUGAAGU | 46 |
| chemically | (HPRT/FVII) | GCGGUGAUGUAAGACUUGAGAUG | |
| unmodified | | AUCCCA | |
| | Strand 2 | GGAUCAUCUCAAGUCUUACAUCAC | 47 |
| | (FVII/mTTR) | CCGUCUAUUAUAGAGCAAGAACAC | |
| | | UGCA | |
| | Strand 3 | CAGUGUUCUUGCUCUAUAAUAGA | 48 |
| | (mTTR/HPRT) | CGGCACUUCAAAUCCAACAAAGUC | |
| | | UGGCA | |
| Y-RNA in | Strand 1 | CCAGACUUUGUUGGAUUUGAAGU | 31 |
| P(+1)P site- | (HPRT/FVII) | GCGGUGAUGUAAGACUUGAGAUG | |
| specific | | AUCCCA | |
| modification | Strand 2 | GGAUCAUCUCAAGUCUUACAUCAC | 32 |
| | (FVII/mTTR) | CCGUCUAUUAUAGAGCAAGAACA | |
| | | CUGCA | |
| | Strand 3 | CAGUGUUCUUGCUCUAUAAUAGA | 33 |
| | (mTTR/HPRT) | CGGCACUUCAAAUCCAACAAAGU | |
| | | CUGGCA | |
| Y-RNA (nick) | Strand 1 | CCAGACUUUGUUGGAUUUGAAGU | 46 |
| chemically | (HPRT/FVII) | GCGGUGAUGUAAGACUUGAGAUG | |
| unmodified | | AUCCCA | |
| | Strand 2 | GGAUCAUCUCAAGUCUUACAUCAC | 47 |
| | (FVII/mTTR) | CCGUCUAUUAUAGAGCAAGAACAC | |
| | | UGCA | |
| | Strand 3 | CAGUGUUCUUGCUCUAUAAUAGA | 39 |
| | (mTTR sense) | CG | |
| | Strand 4 | GCACUUCAAAUCCAACAAAGUCUG | 2 |
| | (HPRT,antisense) | GCA | |
| Y-RNA (nick) | Strand 1 | CCAGACUUUGUUGGAUUUGAAGU | 31 |
| in P(+1)P | (HPRT/FVII) | GCGGUGAUGUAAGACUUGAGAUG | |
| site-specific | | AUCCCA | |
| modification | Strand 2 | GGAUCAUCUCAAGUCUUACAUCAC | 32 |
| | (FVII/mTTR) | CCGUCUAUUAUAGAGCAAGAACA | |
| | | CUGCA | |
| | Strand 3 | CAGUGUUCUUGCUCUAUAAUAGA | 34 |
| | (mTTR sense) | CG | |
| | Strand 4 | GCACUUCAAAUCCAACAAAGUCU | 28 |
| | (HPRT,antisense) | GGCA | |

The sense strands and antisense strands were each mixed in an amount of 500 pmoles and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare siRNA and linear dsRNA.

The Y-RNA construct or the Y-RNA construct with a nick therein were prepared by mixing 100 pmoles of each of three or four strands and hybridizing the strands into double strands through incubation starting at 95° C. for 3 minutes, with a subsequent temperature decrease from 95° C. to 4° C. at a rate of −1.0° C./s in a thermal cycler (Bio-Rad T100™).

### Example 7-2

### Immune Assay

In order to examine the effect of reducing immune induction of the Dicer substrate RNAs modified in the P(+1)P site-specific manner, lipid nanopartides containing the Dicer substrate RNAs prepared in Example 7-1 were prepared. In the case of the linear Dicer substrate RNA, the oligos in the three arms of Y-RNA for the respective three targets HPRT, mTTR, and FVII were mixed at a ratio of 1:1:1 so as to reduce the difference in the immune induction effect by sequence.

Lipid nanoparticles were synthesized by mixing an ethanol phase containing lipids with an aqueous phase containing the nucleic acid sample prepared in Example 7-1 at a volume ratio of 1: 3 (ethanol phase: aqueous phase) in 50 mM sodium acetate buffer by pipetting. The ethanol phase contained the ionizable lipids C12-200 (Wuxi AppTec (Shanghai, China)), 1,2-distearoyl-sn-glycero-3-phosphochloine (Avanti Polar Lipids, Alabaster, AL), cholesterol (Sigma), and 1,2-dimyristoyl-sn-glycero-3-phosphoethanolamine-N-[methoxy(polyethylene glycol)-2000] (ammonium salt) (Avanti) at a molar ratio of 50:10:38.5:1.5, with the weight ratio of C12-200 and the nucleic acid amounting to 5:1.

The concentration of the prepared lipid nanoparticles was measured through Ribogreen assay. RiboGreen analysis was performed using Quant-iT™ RiboGreen® RNA Reagent and Kit (Invitrogen) according to the manufacturer's manual, and the LNP encapsulation efficiency was calculated to determine the final amount of LNP to be applied to the cells.

Human Peripheral blood mononuclear cells (PBMCs) purchased from ATCC were seeded in an amount of 450 μl at a density of 5×10⁵ cells/well into 24-well plates containing an RPMI medium (10% fetal bovine serum, 1% penicillin/streptomycin). After the cells were stabilized by incubation at 37° C. for 24 hours in a 5% CO2 condition, each well was treated with 50 μl of the lipid nanoparticles prepared above to form a final concentration of 100 nM, so that the PBMCs were transfected therewith. Cells from wells treated with only 50 μl of medium and wells treated with only lipid components lacking the nucleic acids were used as controls. In this regard, the number of wells treated per each Dicer substrate RNA sample was n=3, for statistical significance in cytokine quantitative analysis.

After an additional 24 hours of incubation at 37° C. in a 5% CO₂ condition, the cell culture was harvested and centrifuged at 400 g and 4° C. for 5 min. The supernatant thus obtained was quantitatively analyzed for human TNF-α and IFN-α, using an enzyme immunoassay. For cytokine analysis of the supernatant, Human TNF-alpha Quantikine ELISA Kit (R&D system) and Human IFN Alpha ELISA Kit (PBL Assay Science) were used according to the manufacturer's manual, followed by reading absorbance at 450 nm on Infinite M200 Pro (Tecan). One-way ANOVA analysis (Graphpad Prism 6) was used for the comparison of immune induction between non-modification conventional siRNA (19+2) and the other Dicer substrate RNAs and position-specific modification Y-RNAs. The results are depicted in FIG. 12.

As shown in FIG. 12, the linear construct (P(+1)P), Y-RNA construct (P(+1)P Y-RNA), and Y-RNA construct with a nick (P(+1)P Y-RNA(nick)), each having a site-specific chemical modification introduced into the dsRNA substrate according to an embodiment, all decreased is in immune induction, compared to the chemically unmodified constructs (NN, NN Y-RNA, and NN Y-RNA(nick)). From the data, it was understood that the Dicer substrate RNAs with P(+1)P site-specific chemical modification increased in in-vivo stability and decreased in immune induction.

### Example 8

### Dicer Substrate Linear RNA with P(+1)P Site-Specific Chemical Modification

### Example 8-1

### Construction of Chemically Modified Dicer substrate RNA

In order to construct Dicer substrate RNAs capable of being produced into cleaved dsRNAs, sense and antisense strands having the sequences shown in Tables 11 and 10, below, with chemically modified nucleotides at the indicated positions, and siRNAs were synthesized in and purchased from BIONEER. In Tables 11 and 12, below, underlined nucleotides in bold had the sugar ring moiety in which the 2′-OH group was modified into a 2′-O-methyl group. Table 11 lists the sequences forthe linear Dicer substrate RNA constructs to be examined for in vitro gene silencing activity, and Table 12 shows the sequences for the linear Dicer substrate RNA constructs to be examined for in vivo gene silencing activity.

TABLE 11

| Strand | | | Sequence (5′→3′) | SEQ ID NO: |
|---|---|---|---|---|
| HPRT | RNA without | Sense | CCAGACUUUGUUGGAUUUGAAG | 1 |
| target | chemical | | UGC | |
| | modification | Antisense | GCACUUCAAAUCCAACAAAGUC | 2 |
| | | | UGGCA | |
| | RNA with P(+1)P | Sense | CCAGACUUUGUUGGAUUUGAAG | 27 |
| | modification | | UGC | |
| | | Antisense | GCACUUCAAAUCCAACAAAGUC | 28 |
| | | | UGGCA | |
| eGFP | Linear RNA | Sense | GCAAGCUGACCCUGAAGUUAUC | 6 |
| target | without chemical | | ACC | |
| | modification | Antisense | GGUGAUAACUUCAGGGUCAGCU | 7 |
| | | | UGCCA | |
| | Linear RNA with | Sense | GCAAGCUGACCCUGAAGUUAUC | 26 |
| | P(+1)P | | ACC | |
| | modification | Antisense | GGUGAUAACUUCAGGGUCAGCU | 25 |
| | | | UGCCA | |
| TP53 | Linear RNA | Sense | ACCAGGGCAGCUACGGUUUAAG | 49 |
| target | without chemical | | UGC | |
| | modification | Antisense | GCACUUAAACCGUAGCUGCCCU | 50 |
| | | | GGUCA | |
| | Linear RNA with | Sense | ACCAGGGCAGCUACGGUUUAAG | 51 |
| | P(+1)P | | UGC | |
| | modification | Antisense | GCACUUAAACCGUAGCUGCCCU | 52 |
| | | | GGUCA | |

TABLE 12

| Strand | | | Sequence (5′→3′) | SEQ ID NO: |
|---|---|---|---|---|
| FVII target | RNA without | Sense | GGAUCAUCUCAAGUCUUACAUCACC | 41 |
| | chemical | | | |
| | modification | Antisense | GGUGAUGUAAGACUUGAGAUGAUCCCA | 42 |
| | Linear RNA | Sense | GGAUCAUCUCAAGUCUUACAUCACC | 44 |
| | with P(+1)P | | | |
| | modification | Antisense | GGUGAUGUAAGACUUGAGAUGAUCCCA | 45 |
| Mouse TTR | RNA without | Sense | CAGUGUUCUUGCUCUAUAAUAGACG | 39 |
| target | chemical | | | |
| | modification | Antisense | CGUCUAUUAUAGAGCAAGAACACUGCA | 40 |
| | Linear RNA | Sense | CAGUGUUCUUGCUCUAUAAUAGACG | 34 |
| | with P(+1)P | | | |
| | modification | Antisense | CGUCUAUUAUAGAGCAAGAACACUGCA | 43 |

The sense strands and antisense strands were each mixed at an equimolar ratio and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare Dicer substrate RNAs.

### Example 8-2

### Assay for Gene Silencing Effect

(In-vitro RT-PCR Assay for Gene Silencing Effect)—HRRT and TP53 Targets

The linear Dicer substrate RNAs (Table 11) prepared in Example 8-1 were examined to for gene silencing effect on the target genes HRRT and TP53 by RT-PCR in a similar manner to that in Example 1-2. The results are depicted in Table 13. Primer sequences for TP53 are listed in Table 13, below.

TABLE 13

| Name | Sequence (5′→3′) | SEQ ID NO: |
|---|---|---|
| TP53_Foward | TTCCGAGAGCTGAATGAGGC | SEQ ID NO: 53 |
| TP53_Reverse | GAAGTGGAGAATGTCAGTCTGAGTC | SEQ ID NO: 54 |

(In-Vitro FACS Assay for Gene Silencing Effect)—GFP Target

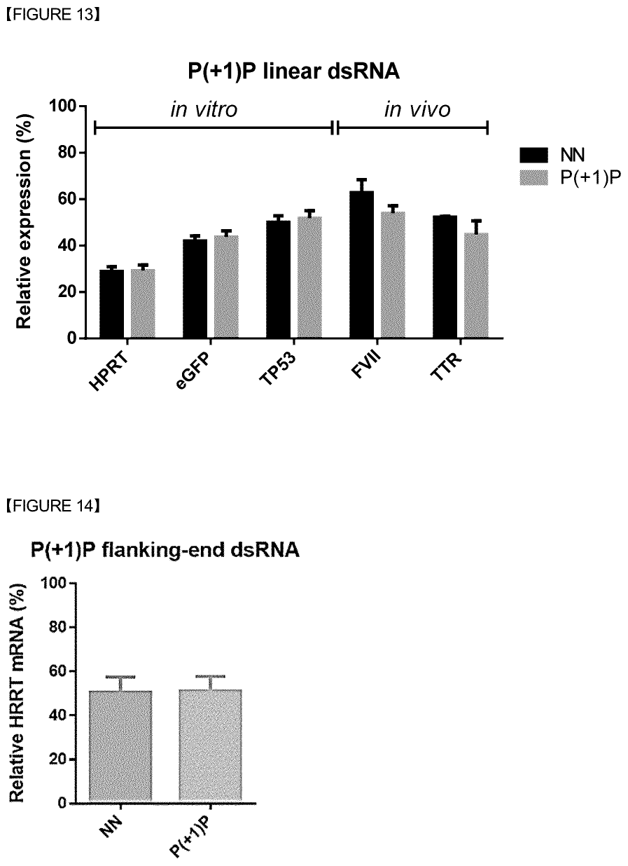

The linear Dicer substrate RNAs (Table 11) prepared in Example 8-1 were applied to GFP-KB cells, and the expression level of the target gene GFP was measured using FACS in a similar manner to that in Example 1-3, and the results are depicted in FIG. 13.

(In-Vivo ELISA Assay for Gene Silencing Effect)—TTR Target

Lipid nanopartides containing the Dicer substrate RNAs prepared in Example 8-1 were prepared. Lipid nanoparticles were synthesized by mixing an ethanol phase containing lipids with an aqueous phase containing the nudeic acid sample prepared in Example 7-1 at a volume ratio of 1: 3 (ethanol phase: aqueous phase) in 50 mM sodium acetate buffer. The ethanol phase contained the ionizable lipids 012-200 (Wuxi AppTec (Shanghai, China)), 1,2-distearoyl-sn-glycero-3-phosphochloine (Avanti Polar Lipids, Alabaster, AL), cholesterol (Sigma), and 1,2-dimyristoyl-sn-glycero-3-phosphoethanolamine-N-[methoxy(polyethylene glycol)-2000] (ammonium salt) (Avanti) at a molar ratio of 50:10:38.5:1.5, with the weight ratio of 012-200 and the nucleic acid amounting to 5:1. C57bl/6NCrSlc mice (Charles River Labs, female, 18-22 g) 6 weeks old were given a tail IV injection of the lipid nanoparticles prepared above at a dose of 0.01 mg/kg. Each sample was injected into three mice. A control for mTTR analysis was composed of three mice. For use in normalization when calculating TTR expression rate, three mice were injected with PBS. Seventy-two hours after injection, mouse plasma was obtained by collecting blood from the cheek of the mice. Mouse blood TTR concentrations were quantitated using a Mouse Prealbumin ELISA kit (ALPCO). The plasma samples were diluted and reacted according to the manufacturer's manual, followed by reading absorbance at 450 nm on Infinite M200 Pro (Tecan) to quantify TTR concentrations, and the results are depicted in FIG. 13. One-way ANOVA analysis (Graphpad Prism 6) was performed to examine the influence of site-specific modification on gene silencing.

(In-Vivo Chromogenic Assay for Gene Silencing Effect)—FVII Target

C57bl/6NCrSlc (Charles River Labs, female, 18-22g) with 6 weeks of age were regarded to weigh 20 g. To the animals, the RNA samples (FVII target) formulated with lipid nanoparticles were administered at a dose of 100 μl containing RNA 0.03 mg/kg by tail IV injection. Each sample was injected into three mice. A control for FVII analysis to obtain a standard curve was composed of three mice. As a positive control, three mice were injected with PBS. Seventy-two hours after injection, mouse plasma was obtained by collecting blood from the cheek of the mice. Mouse blood FVII expression levels were analyzed using a COASET Factor VII kit (Chromogenix). Absorbance was read at 450 nm on Infinite M200 Pro (Tecan) to analyze FVII expression levels, and the results are depicted in FIG. 13. The significance of the difference in in-vivo gene silencing effect between the control (chemically unmodified Dicer substrate RNAs) and Dicer substrate RNAs with (P+)P site-specific chemical modification was verified by one-way ANOVA analysis (Graphpad Prism 6).

As shown in FIG. 13, the gene-silencing activity of the dsRNA with site-specific chemical modification according to an embodiment was not decreased in vitro, but increased in vivo, compared to the control. In addition, similar results were obtained even when sequences targeting various genes were included, it is interpreted that there is no effect depending on the gene sequence in the site-specific chemical method according to an embodiment.

### Example 9

### Gene Silencing Effect of Flanking End dsRNA with P(+1)P Site-Specific Chemical Modification

In this Example, flanking-end Dicer substrate RNAs with P(+1)P site-specific chemical modification were examined for gene silencing effect. Flanking-end Dicer substrate RNA 25 constructs are illustratively depicted in FIG. 9.

### Example 9-1

### Construction of dsRNA with Chemical Modification

To construct flanking-end Dicer substrate RNAs capable of being prepared into deaved dsRNAs, sense and antisense strands having the sequences shown in Table 14, below, with chemically modified nudeotides at the indicated positions, were synthesized in and purchased from BIONEER. In Table 14, below, underlined nucleotides in bold had the sugar ring moiety in which the 2′-OH group was modified into a 2′-O-methyl group.

TABLE 14

| Strand | | Sequence (5′→3′) | SEQ ID NO: |
|---|---|---|---|
| Flanking end | Strand 1 | CCAGACUUUGUUGGAUUUGAAGUGCGGUGAUG | 46 |
| dsRNA without | (HPRT/FVII) | UAAGACUUGAGAUGAUCCCA | |
| chemical | Strand 2 | CAGUGUUCUUGCUCUAUAAUAGACGGCACUUC | 48 |
| modification | (mTTR/HPRT) | AAAUCCAACAAAGUCUGGCA | |
| Flanking end | Strand 1 | CCAGACUUUGUUGGAUUUGAAGUGCGGUGAUG | 31 |
| dsRNA with | (HPRT/FVII) | UAAGACUUGAGAUGAUCCCA | |
| P(+1)P site- | | | |
| specific | Strand 2 | CAGUGUUCUUGCUCUAUAAUAGACGGCACUUC | 33 |
| modification | (mTTR/HPRT) | AAAUCCAACAAAGUCUGGCA | |

Strand 1 and strand 2 were each mixed in an amount of 100 pmoles and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare flanking-end dsRNA.

### Example 9-2

### In-Vitro RT-PCR Assay For Gene Silencing Effect

The flanking-end Dicer substrate RNAs prepared in Example 9-1 were applied to GFP-KB cells in the same manner as in Example 1-2, and the silencing effect on HRRT, a target gene, in vitro was measured through RT-PCR. The results are shown in the right plot of FIG. 14. The significance was verified through correlation among the chemically unmodified control and the flanking-end Dicer substrate RNA group with P(+1)P site-specific chemical modification by Ordinary one-way ANOVA analysis (Graphpad Prism 6).

As shown in FIG. 14, similar gene silencing effects were detected in vitro between the control and the flanking-end Dicer substrate dsRNA with P(+1)P site-specific chemical modification.

### Example 10

### In-vivo Gene Silencing Effect of Y-RNA Construct with P(+1)P Site-Specific Chemical Modification

In this Example, examination was made of the in-vivo silencing effect of a Dicer substrate Y-RNA construct with P(+1)P site-specific chemical modification. The Y-RNA structure is exemplified as shown in FIG. 9.

### Example 10-1

### Construction of Chemically Modified Dicer Substrate dsRNA

To construct flanking-end Dicer substrate RNAs capable of being prepared into deaved dsRNAs, strands having the sequences shown in Table 15, below, with chemically modified nucleotides at the indicated positions, were synthesized in and purchased from BIONEER. In Table 15, below, underlined nucleotides in bold had the sugar ring moiety in which the 2′-OH group was modified into a 2′-O-methyl group.

TABLE 15

| Strand | | Sequence (5′→3′) | SEQ ID NO: |
|---|---|---|---|
| Y-RNA without | Strand 1 | CCAGACUUUGUUGGAUUUGAAGUGCGGUGAUGUA | 46 |
| chemical | (HPRT/FVII) | AGACUUGAGAUGAUCCCA | |
| modification | Strand 2 | GGAUCAUCUCAAGUCUUACAUCACCCGUCUAUUA | 47 |
| | (FVII/mTTR) | UAGAGCAAGAACACUGCA | |
| | Strand 3 | CAGUGUUCUUGCUCUAUAAUAGACGGCACUUCAA | 48 |
| | (mTTR/HPRT) | AUCCAACAAAGUCUGGCA | |
| Y-RNA with | Strand 1 | CCAGACUUUGUUGGAUUUGAAGUGCGGUGAUGUA | 31 |
| P(+1)P site- | (HPRT/FVII) | AGACUUGAGAUGAUCCCA | |
| specific chemical | Strand 2 | GGAUCAUCUCAAGUCUUACAUCACCCGUCUAUUA | 32 |
| modification | (FVII/mTTR) | UAGAGCAAGAACACUGCA | |
| | Strand 3 | CAGUGUUCUUGCUCUAUAAUAGACGGCACUUCAA | 33 |
| | (mTTR/HPRT) | AUCCAACAAAGUCUGGCA | |

The three strands of Table 15 were each mixed in an amount of 1 nmole and hybridized into double strands by incubation starting at 95° C. for 3 minutes, with a subsequent temperature decrease from 95° C. to 4° C. at a rate of −1.0° C./s in a thermal cycler (Bio-Rad T100 T″'), to prepare Y-RNA constructs.

### Example 10-2

### In-Vivo Assay for Gene Silencing Effect

To C57bl/6NCrSlc mice (Charles River Labs, female, 18-22 g) with 6 weeks of age, the Dicer substrate Y-RNA constructs prepared in Example 10-1 were administered at a dose of 100 μl containing RNA 0.03 mg/kg by tail IV injection.

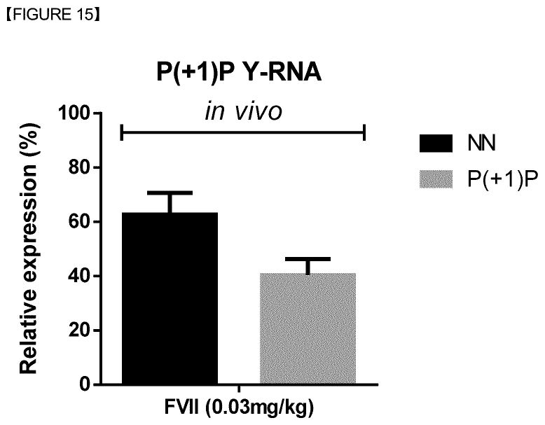

Each sample was injected into three mice. A standard curve for FVII analysis was plotted on the basis of data from three mice. As a positive control, three mice were injected with PBS. Seventy-two hours after injection, mouse plasma was obtained by collecting blood from the cheek of the mice. Mouse blood FVII expression levels were analyzed using a COASET Factor VII kit (Chromogenix). Absorbance was read at 450 nm on Infinite M200 Pro (Tecan) to analyze FVII expression levels, and the results are depicted in FIG. 15. The significance of the difference in in-vivo gene silencing effect between the control (Dicer substrate RNA without chemical modification) and Dicer substrate RNAs with (P+)P site-specific chemical modification was verified by one-way ANOVA analysis (Graphpad Prism 6).

As shown in FIG. 15, the Y-RNA construct with site-specific chemical modification according to an embodiment exhibited an increased gene silencing effect in vivo.

### Example 11

### In-Vivo Gene Silencing Effect of Y-RNA Construct with P(+1)P Site-Specific Chemical Modification and Nick

In this Example, examination was made of the in-vivo silencing effect of a Dicer substrate Y-RNA construct including a P(+1)P site-specific chemical modification and a nick. to The Dicer substrate Y-RNA structure with a nick is exemplified as shown in FIG. 9.

### Example 11-1

### Construction of Dicer Substrate dsRNA with Chemical Modification

To construct Dicer substrate Y-RNA with a nick therein, which could be prepared into deaved dsRNA, strands having the sequences shown in Table 16, below, with chemically modified nucleotides at the indicated positions, were synthesized in and purchased from BIONEER. In Table 16, below, underlined nucleotides in bold had the sugar ring moiety in which the 2′-OH group was modified into a 2′-O-methyl group.

TABLE 16

| Strand | | Sequence (5′→3′) | SEQ ID NO: |
|---|---|---|---|
| Y-RNA (nick) | Strand 1 | CCAGACUUUGUUGGAUUUGAAGUGCGGUGAUG | 46 |
| without chemical | (HPRT/FVII) | UAAGACUUGAGAUGAUCCCA | |
| modification | Strand 2 | GGAUCAUCUCAAGUCUUACAUCACCCGUCUAU | 47 |
| | (FVII/mTTR) | UAUAGAGCAAGAACACUGCA | |
| | Strand 3 | CAGUGUUCUUGCUCUAUAAUAGACG | 39 |
| | (mTTR sense) | | |
| | Strand 4 | GCACUUCAAAUCCAACAAAGUCUGGCA | 2 |
| | (HPRT antisense) | | |
| Y-RNA (nick) with | Strand 1 | CCAGACUUUGUUGGAUUUGAAGUGCGGUGAUG | 31 |
| P(+1)P site- | (HPRT/FVII) | UAAGACUUGAGAUGAUCCCA | |
| specific chemical | Strand 2 | GGAUCAUCUCAAGUCUUACAUCACCCGUCUAU | 32 |
| modification | (FVII/mTTR) | UAUAGAGCAAGAACACUGCA | |
| | Strand 3 | CAGUGUUCUUGCUCUAUAAUAGACG | 34 |
| | (mTTR sense) | | |
| | Strand 4 | GCACUUCAAAUCCAACAAAGUCUGGCA | 28 |
| | (HPRT antisense) | | |

The sense strands and antisense strands were each mixed in an amount of 1 nmole and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare Y-RNA construct with a nick.

### Example 11-2

### In-Vivo Assay for Gene Silencing Effect

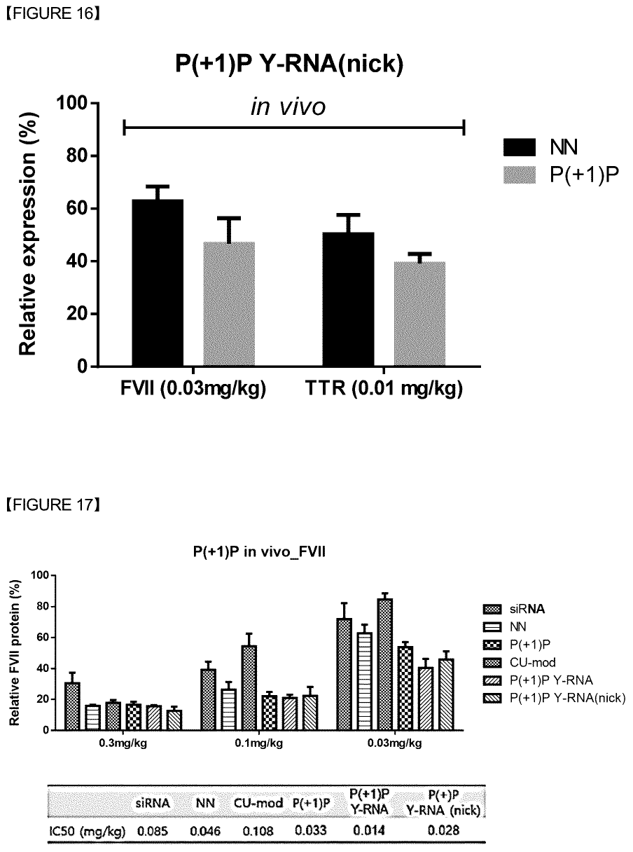

Lipid nanopartides containing the Dicer substrate Y-RNA construct prepared in Example 10-1 were synthesized in a similar manner to that of Example 8-2 and administered at a dose of 100 μl containing RNA 0.03 mg/kg by tail IV injection into C57bl/6NCrSlc mice (Charles River Labs, female, 18-22g) with 6 weeks of age. Expression levels of FVII and TTR were measured using the chromogenic assay and ELISA method as in Example 8-2, and the results are depicted in FIG. 16.

As shown in FIG. 16, both the Y-RNA constructs with a site-specific chemical modification and a nick according to an embodiment exhibited an increased gene silencing effect in vivo, irrespective of the sequences (targets).

### Example 12

### In-Vito Gene Silencing Effect According to Concentration of Dicer Substrate RNA with P(+1)P Site-Specific Chemical Modification

In this Example, a Dicer substrate Y-RNA construct and a Y-RNA construct with a nick therein, both having P(+1)P site-specific chemical modifications made thereto, were prepared and examined for gene silencing effects in vivo according to concentrations thereof. The Y-RNA constructs are exemplified as shown in FIG. 9.

### Example 12-1

### Construction of Chemically Modified Dicer Substrate dsRNA

To construct Dicer substrate RNAs in various structures (linear dsRNA, Y-RNA, and Y-RNA with a nick), which could be prepared into cleaved dsRNAs, strands having the sequences shown in Tables 17 and 18, below, with chemically modified nudeotides at the indicated positions, were synthesized in and purchased from BIONEER. In Tables 17 and 18, below, underlined nucleotides in bold had the sugar ring moiety in which the 2′-OH group was modified into a 2′-O-methyl group. The strands listed in Table 17 were designed to construct siRNAs and Dicer substrate RNAs, all targeting FVII while the strands listed in Table 18 were designed to construct siRNAs and Dicer substrate RNAs, all targeting TTR.

TABLE 17

| Strand | | Sequence (5′→3′) | SEQ ID NO: |
|---|---|---|---|
| siRNA (19 + 2) | Sense strand | GGAUCAUCUCAAGUCUUACTT | 37 |
| | Antisense | GUAAGACUUGAGAUGAUCCTT | 38 |
| | strand | | |
| Dicer substrate | Sense strand | GGAUCAUCUCAAGUCUUACAUCACC | 41 |
| linear RNA (NN) | Antisense | GGUGAUGUAAGACUUGAGAUGAUCCCA | 42 |
| without chemical | strand | | |
| modification | | | |
| Dicer substrate | Sense strand | GGAUCAUCUCAAGUCUUACAUCACC | 44 |
| linear RNA with | Antisense | GGUGAUGUAAGACUUGAGAUGAUCCCA | 45 |
| P(+1)P site-specific | strand | | |
| modification | | | |
| C/U sequence- | Sense strand | GGAUCAUCUCAAGUCUUACAUCACC | 55 |
| based modification | Antisense | GGUGAUGUAAGACUUGAGAUGAUCCCA | 56 |
| | strand | | |
| Y-RNA with P(+1)P | Strand 1 | CCAGACUUUGUUGGAUUUGAAGUGCGG | 31 |
| site-specific | (HPRT/FVII) | UGAUGUAAGACUUGAGAUGAUCCCA | |
| modification | Strand 2 | GGAUCAUCUCAAGUCUUACAUCACCCG | 32 |
| | (FVII/mTTR) | UCUAUUAUAGAGCAAGAACACUGCA | |
| | Strand 3 | CAGUGUUCUUGCUCUAUAAUAGACGGC | 33 |
| | (mTTR/HPRT) | ACUUCAAAUCCAACAAAGUCUGGCA | |
| Y-RNA (nick) with | Strand 1 | CCAGACUUUGUUGGAUUUGAAGUGCGG | 31 |
| P(+1)P site-specific | (HPRT/FVII) | UGAUGUAAGACUUGAGAUGAUCCCA | |
| modification | Strand 2 | GGAUCAUCUCAAGUCUUACAUCACCCG | 32 |
| | (FVII/mTTR) | UCUAUUAUAGAGCAAGAACACUGCA | |
| | Strand 3 | CAGUGUUCUUGCUCUAUAAUAGACG | 34 |
| | (mTTR sense) | | |
| | Strand 4 | GCACUUCAAAUCCAACAAAGUCUGGCA | 28 |
| | (HPRT antisense) | | |

TABLE 18

| Strand | | Sequence (5′→3′) | SEQ ID NO: |
|---|---|---|---|
| SIRNA (19 + 2) | Sense strand | CAGUGUUCUUGCUCUAUAATT | 35 |
| | Antisense strand | UUAUAGAGCAAGAACACUGTT | 36 |
| Dicer substrate | Sense strand | CAGUGUUCUUGCUCUAUAAUAGACG | 39 |
| linear RNA (NN) | Antisense strand | CGUCUAUUAUAGAGCAAGAACACUGCA | 40 |
| without | | | |
| chemical | | | |
| modification | | | |
| Y-RNA (nick) | Strand 1 | CCAGACUUUGUUGGAUUUGAAGUGCGG | 31 |
| with P(+1)P | (HPRT/FVII) | UGAUGUAAGACUUGAGAUGAUCCCA | |
| site-specific | Strand 2 | GGAUCAUCUCAAGUCUUACAUCACCCG | 32 |
| modification | (FVII/mTTR) | UCUAUUAUAGAGCAAGAACACUGCA | |
| | Strand 3(mTTR, | CAGUGUUCUUGCUCUAUAAUAGACG | 34 |
| | sense) | | |
| | Strand 4 | GCACUUCAAAUCCAACAAAGUCUGGCA | 28 |
| | (HPRT,antisense) | | |

The sense strands and antisense strands were each mixed in an amount of 10 nmoles and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare siRNA and linear dsRNA.

The Y-RNA construct or the Y-RNA construct with a nick therein were prepared by mixing 100 pmoles of each of three or four strands and hybridizing the strands into double strands through incubation starting at 95° C. for 3 minutes, with a subsequent temperature decrease from 95° C. to 4° C. at a rate of −1.0° C./s in a thermal cycler (Bio-Rad T100™).

### Example 12-2

### In-Vivo Gene Silencing Effect According to Concentration

Lipid nanoparticles containing the various Dicer substrate Y-RNA constructs prepared in Example 12-1 were synthesized in a similar manner to that of Example 8-2 and administered ata dose of 100 μl containing various amounts of the RNA samples (0.03 mg/kg is to 0.3 mg/kg) by tail IV injection into C57bl/6NCrSlc mice (Charles River Labs, female, 18-22 g) with 6 weeks of age. In-vivo gene silencing effects against the TTR target were examined in the siRNA as a control, the linear dsRNA without any chemical modification, the linear dsRNA with a P(+1)P site-specific chemical modification, and the Y-RNA with a P(+1)P site-specific chemical modification and a nick.

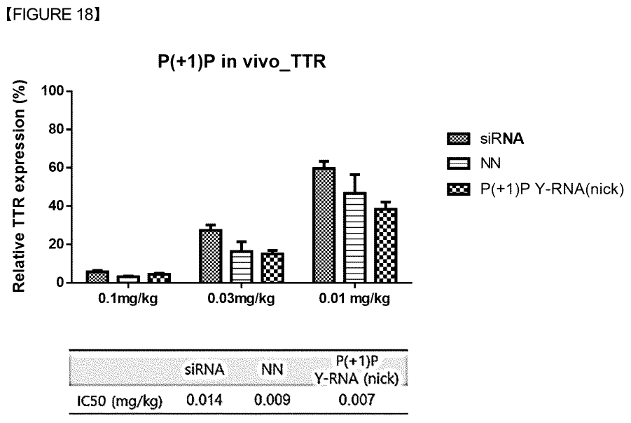

Expression levels of FVII and TTR were measured using the chromogenic assay and ELISA method as in Example 8-2, and the results are depicted in FIGS. 17 and 18, respectively. In addition, IC50 values accounting for inhibiting the expression of FVII and TTR in each control group and experimental group were calculated, and the results are tabulated in FIGS. 17 and 18, respectively.

As shown in FIGS. 17 and 18, the linear Dicer substrate dsRNA (P(+1)P), the Y-RNA construct (P(+1)P Y-RNA), and the Y-RNA construct with a nick (P(+1)P Y-RNA(nick)), each having a P(+1)P site-specific modification made thereto, were all observed to exhibit higher inhibitory effects against the expression of the target protein in vivo, with significantly lower IC50 values, than the control (NN), and these effects were consistent irrespective of the sequences (targets).

### Example 13

### Gene Silencing Effect According to P(+1)P Site-Specific Chemical Modification Position

In this Example, in the context of the P(+1)P site-specific chemical modification responsible for modifications made to the nucleotides on the sense strand at 1^{st}, 4^{th}, 5^{th}, 7^{th}, and 14^{th} positions from the 5′ end thereof, the nucleotides on the antisense strand at corresponding positions to 2^{nd}, 3^{rd}, 6^{th}, 8^{th}, and 10^{th} to 13^{th} positions from the 5′ end of the sense strand complementary thereto, the effect of chemical modification at each position of the sense strand on gene silencing activity was examined.

### Example 13-1

### Preparation of Y-RNA Structure with P(+1)P Site-Specific Chemical Modification And Nick

To construct Y-RNA with a nick, capable of being prepared into cleaved dsRNA, strands having the sequences shown in Table 19, below, with chemically modified nucleotides at the indicated positions, were synthesized in and purchased from BIONEER. In Table 19, below, underlined nucleotides in bold had the sugar ring moiety in which the 2′-OH group was modified into a 2′-O-methyl group.

TABLE 19

| Strand | | Sequence (5′→3′) | SEQ ID NO: |
|---|---|---|---|
| Position | Strand 1 | CCAGACUUUGUUGGAUUUGAAGU | 31 |
| modified (PP) | (HPRT/FVII) | GCGGUGAUGUAAGACUUGAGAUG | |
| Y-RNA (nick) | | AUCCCA | |
| | Strand 2 | GGAUCAUCUCAAGUCUUACAUCA | 32 |
| | (FVII/mTTR) | CCCGUCUAUUAUAGAGCAAGAAC | |
| | | ACUGCA | |
| | Strand 3 | CAGUGUUCUUGCUCUAUAAUAGA | 34 |
| | (mTTR sense) | CG | |
| | Strand 4 | GCACUUCAAAUCCAACAAAGUCU | 28 |
| | (HPRT, antisense) | GGCA | |
| Position | Strand 1 | CCAGACUUUGUUGGAUUUGAAGU | 31 |
| modified (-4) | (HPRT/FVII) | GCGGUGAUGUAAGACUUGAGAUG | |
| Y-RNA (nick) | | AUCCCA | |
| | Strand 2 | GGAUCAUCUCAAGUCUUACAUCA | 32 |
| | (FVII/mTTR) | CCCGUCUAUUAUAGAGCAAGAAC | |
| | | ACUGCA | |
| | Strand 3 | CAGUGUUCUUGCUCUAUAAUAGA | 57 |
| | (mTTR sense) | CG | |
| | Strand 4 | GCACUUCAAAUCCAACAAAGUCU | 28 |
| | (HPRT, antisense) | GGCA | |
| Position | Strand 1 | CCAGACUUUGUUGGAUUUGAAGU | 31 |
| modified (-5) | (HPRT/FVII) | GCGGUGAUGUAAGACUUGAGAUG | |
| Y-RNA (nick) | | AUCCCA | |
| | Strand 2 | GGAUCAUCUCAAGUCUUACAUCA | 32 |
| | (FVII/mTTR) | CCCGUCUAUUAUAGAGCAAGAAC | |
| | | ACUGCA | |
| | Strand3 | CAGUGUUCUUGCUCUAUAAUAGA | 58 |
| | (mTTR sense) | CG | |
| | Strand 4 | GCACUUCAAAUCCAACAAAGUCU | 28 |
| | (HPRT, antisense) | GGCA | |
| Position | Strand 1 | CCAGACUUUGUUGGAUUUGAAGU | 31 |
| modified (-7) | (HPRT/FVII) | GCGGUGAUGUAAGACUUGAGAUG | |
| Y-RNA (nick) | | AUCCCA | |
| | Strand 2 | GGAUCAUCUCAAGUCUUACAUCA | 32 |
| | (FVII/mTTR) | CCCGUCUAUUAUAGAGCAAGAAC | |
| | | ACUGCA | |
| | Strand 3 | CAGUGUUCUUGCUCUAUAAUAGA | 59 |
| | (mTTR sense) | CG | |
| | Strand 4 | GCACUUCAAAUCCAACAAAGUCU | 28 |
| | (HPRT, antisense) | GGCA | |
| Position | Strand 1 | CCAGACUUUGUUGGAUUUGAAGU | 31 |
| modified (-14) | (HPRT/FVII) | GCGGUGAUGUAAGACUUGAGAUG | |
| Y-RNA (nick) | | AUCCCA | |
| | Strand 2 | GGAUCAUCUCAAGUCUUACAUCA | 32 |
| | (FVII/mTTR) | CCCGUCUAUUAUAGAGCAAGAAC | |
| | | ACUGCA | |
| | Strand 3 | CAGUGUUCUUGCUCUAUAAUAGA | 60 |
| | (mTTR sense) | CG | |
| | Strand 4 | GCACUUCAAAUCCAACAAAGUCU | 28 |
| | (HPRT, antisense) | GGCA | |

The four strands of Table 19 were each mixed in an amount of 1 nmole and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare Y-RNA constructs with a nick. The prepared Y-RNA constructs with a nick had three arms that could be prepared into cleaved dsRNA by Dicer cleavage. The Y-RNA constructs with a nick therein bear P(+1)P site-specific modifications in each arm thereof. Among the Y-RNA constructs bearing (+1)P site-specific modifications were groups with no chemical modifications at 4^{th}, 5^{th}, 7^{th} or 14^{th} from the 5′ end on the sense strand (named P(−4)P, P(−5)P, P(−7)P, and P(−14)P, respectively).

### Example 13-2

### In-Vivo Gene Silencing Effect

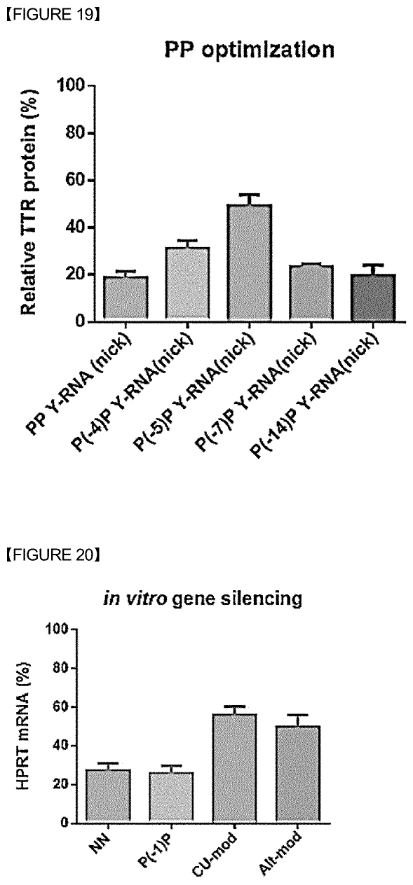

Lipid nanoparticles containing the Dicer substrate Y-RNA construct with a nick, prepared in Example 12-1, were synthesized in a similar manner to that of Example 8-2 and administered at a dose of 100 μl containing RNA 0.03 mg/kg by tail IV injection into C57bl/6NCrSlc mice (Charles River Labs, female, 18-22g) with 6 weeks of age. Expression levels of TTR were measured using ELISA, and the results are depicted in FIG. 19.

Among the Y-RNA constructs, as shown in FIG. 19, the P(+1)P group with a chemical modification to the nucleotides at 1^{st}, 4^{th}, 5^{th}, 7^{th} and 14^{th} positions from the 5′ end on the sense strand of the arm exhibited the largest in-vivo gene silencing effect while decreased in-vivo gene silencing effects were detected in P(−4)P, P(−5)P, P(−7)P, and P(−14)P groups with no chemical modifications at 4^{th}, 5^{th}, 7^{th} and 14^{th} positions from the 5′ end on the sense strand, respectively.

### Example 14

### Dicer Substrate dsRNA with P(−1)P Site-Specific Chemical Modification

In this Example, a Dicer substrate RNA was constructed by making a chemical modification to nucleotides at 4^{th}, 5^{th}, 7^{th} and 14^{th} positions from the 5′ end on the sense strand in Dicer substrate RNA and to nucleotides on the antisense strand at positions corresponding to 2^{nd}, 3^{rd}, 6^{th}, 8^{th}, and 10^{th} to 13^{th} positions from the 5′ end of the sense strand complementary thereto (hereinafter, referred to as P(−1)P site-specific chemical modification) and examined for gene silencing activity.

### Example 14-1

### Construction of Dicer Substrate dsRNA with Chemical Modification

To construct linear icer substrate RNAs capable of being prepared into cleaved dsRNAs, sense and antisense strands having the sequences shown in Table 20, below, with chemically modified nucleotides at the indicated positions, were synthesized in and purchased from BIONEER. In Table 20, below, underlined nucleotides in bold had the sugar ring moiety in which the 2′-OH group was modified into a 2′-O-methyl group.

TABLE 20

| Strand | | Sequence (5′→3′) | SEQ ID NO: |
|---|---|---|---|
| No chemical | Sense strand | CCAGACUUUGUUGGAUUUGAAGUGC | 1 |
| modification | Antisense | GCACUUCAAAUCCAACAAAGUCUGGCA | 2 |
| (NN) | strand | | |
| P(-1)P site- | Sense strand | CCAGACUUUGUUGGAUUUGAAGUGC | 61 |
| specific | Antisense | GCACUUCAAAUCCAACAAAGUCUGGCA | 62 |
| modification | strand | | |
| C/U sequence- | Sense strand | CCAGACUUUGUUGGAUUUGAAGUGC | 20 |
| based | Antisense | GCACUUCAAAUCCAACAAAGUCUGGCA | 21 |
| modification | strand | | |
| Alternating | Sense strand | CCAGACUUUGUUGGAUUUGAAGUGC | 22 |
| modification | Antisense | GCACUUCAAAUCCAACAAAGUCUGGCA | 23 |
| | strand | | |

The sense strands and antisense strands of Table 20 were each mixed in an amount of 100 pmoles and hybridized into double strands by incubation starting at 95° C. for 3 minutes, with a subsequent temperature decrease from 95° C. to 4° C. at a rate of −1.0° C./s in a thermal to cycler (Bio-Rad T100™), to prepare the following Dicer substrate dsRNAs capable of being prepared into deaved dsRNA targeting HRRT: (1) a chemically unmodified control (NN), (2) Dicer substrate linear dsRNA with a P(−1)P site-spedfic modification (P(−1)P), (3) Dicer substrate linear dsRNA with a C/U sequence-based chemical modification (CU-mod), and (4) Dicer substrate linear dsRNA with an alternating chemical modification (Alt-mod).

### Example 14-2

### RT-PCR Assay for Gene Silencing Effect

The three Dicer substrate RNAs constructed in Example 14-1 were applied to GFP-KB in a similar manner as in Example 1-2 and examined for silencing effects on the target gene HRRT in vitro. The results are depicted in FIG. 20.

Compared to the control, as shown in FIG. 20, the in vitro gene silencing effect was not decreased in the Dicer substrate dsRNA with a P(−1)P site-specific chemical modification, but increased in the Dicer substrate dsRNA with other chemical modifications according to an embodiment.

### Example 15

### Linear dsRNA with P(−1)P Site-Specific Chemical Modification Introduced Thereto

In this Example, linear Dicer substrate RNAs with P(−1)P site-specific chemical modification were constructed, and examined for examined in vitro or in vivo gene silencing is effects

### Example 15-1

### Construction of dsRNA with Site-Specific Chemical Modification

To construct linear Dicer substrate RNAs capable of being prepared into cleaved dsRNAs, strands having the sequences shown in Tables 21 and 22, below, with chemically modified nucleotides at the indicated positions, were synthesized in and purchased from BIONEER. In Tables 21 and 22, below, underlined nucleotides in bold had the sugar ring moiety in which the 2′-OH group was modified into a 2′-O-methyl group. The sequences listed in Tables 21 and 22 were designed to construct linear Dicer substrate RNAs to be examined for in vitro and in vivo gene silencing activity, respectively.

TABLE 21

| Strand | | | Sequence (5′→3′) | SEQ ID NO: |
|---|---|---|---|---|
| HPRT | Linear RNA | Sense | CCAGACUUUGUUGGAUUUGAAGUGC | 1 |
| target | without | Antisense | GCACUUCAAAUCCAACAAAGUCUGGCA | 2 |
| | chemical | | | |
| | modification | | | |
| | Linear RNA with | Sense | CCAGACUUUGUUGGAUUUGAAGUGC | 61 |
| | P(-1)P | Antisense | GCACUUCAAAUCCAACAAAGUCUGGCA | 62 |
| | modification | | | |
| eGFP | Linear RNA | Sense | GCAAGCUGACCCUGAAGUUAUCACC | 6 |
| target | without | Antisense | GGUGAUAACUUCAGGGUCAGCUUGCCA | 7 |
| | chemical | | | |
| | modification | | | |
| | Linear RNA with | Sense | GCAAGCUGACCCUGAAGUUAUCACC | 63 |
| | P(-1)P | Antisense | GGUGAUAACUUCAGGGUCAGCUUGCCA | 64 |
| | modification | | | |

TABLE 22

| Strand | | | Sequence (5′→3′) | SEQ ID NO: |
|---|---|---|---|---|
| FVII target | Linear RNA | Sense | GGAUCAUCUCAAGUCUUACAUCACC | 41 |
| | without | Antisense | GGUGAUGUAAGACUUGAGAUGAUCCCA | 42 |
| | chemical | | | |
| | modification | | | |
| | Linear RNA | Sense | GGAUCAUCUCAAGUCUUACAUCACC | 65 |
| | with P(-1)P | Antisense | GGUGAUGUAAGACUUGAGAUGAUCCCA | 66 |
| | modification | | | |
| Mouse | Linear RNA | Sense | CAGUGUUCUUGCUCUAUAAUAGACG | 39 |
| TTR target | without | Antisense | CGUCUAUUAUAGAGCAAGAACACUGCA | 40 |
| | chemical | | | |
| | modification | | | |
| | Linear RNA | Sense | CAGUGUUCUUGCUCUAUAAUAGACG | 67 |
| | with P(-1)P | Antisense | CGUCUAUUAUAGAGCAAGAACACUGCA | 68 |
| | modification | | | |

The sense strands and antisense strands were each mixed at an equimolar ratio and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare linear Dicer substrate RNAs.

### Example 15-2

### Assay for Gene Silencing Effect

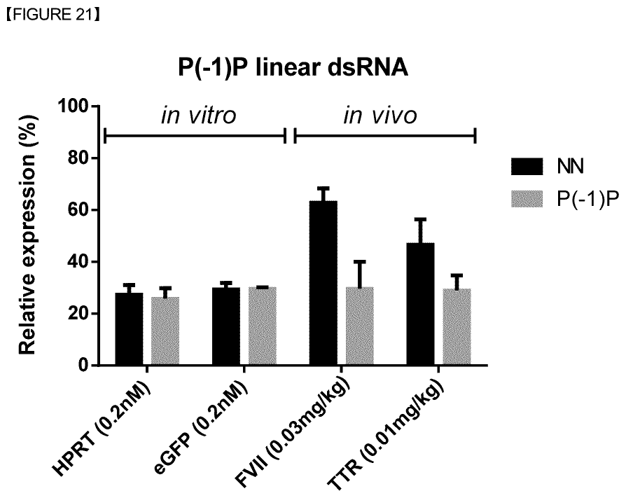

Linear Dicer substrate RNAs with a P(−1)P chemical modification introduced thereinto, constructed in Example 15-1, were measured for in vitro gene silencing activity against HRRT and GFP targets and in vivo gene silencing activity against FVI I and TTR targets in a similar manner to that of Example 8-2, and the results are depicted in FIG. 21. The Dicer substrate RNAs targeting FVII and TTR were injected at doses of 0.03 mg/kg and 0.01 mg/kg to mice, respectively.

Compared to the control, as shown in FIG. 21, linear Dicer substrate RNAs having P(−1)P chemical modifications introduced thereinto did not decrease in gene silencing activity in vitro, but exhibited significantly high gene silencing activity in vivo.

### Example 16

### Y-RNA (Nick) with P(−1)P Site-Specific Chemical Modification Introduced Into

In this Example, Dicer substrate Y-RNAs with a nick therein to which a P(−1)P site-specific chemical modification had been made were examined for gene silencing activity. Dicer substrate Y-RNA constructs with a nick is exemplified as shown in FIG. 9.

### Example 16-1

### Construction of Chemically Modified Dicer Substrate RNA

To construct Dicer substrate Y-RNA with a nick therein which could be prepared into deaved dsRNAs, strands having the sequences shown in Table 23, below, with chemically modified nucleotides at the indicated positions, were synthesized in and purchased from BIONEER. In Table 23, below, underlined nucleotides in bold had the sugar ring moiety in which the 2′-OH group was modified into a 2′-O-methyl group.

TABLE 23

| Strand | | Sequence (5′→3′) | SEQ ID NO: |
|---|---|---|---|
| Y-RNA (nick) | Strand1 | CCAGACUUUGUUGGAUUUGAAGUGCGGUGA | 46 |
| without chemical | (HPRT/FVII) | UGUAAGACUUGAGAUGAUCCCA | |
| modification | Strand2 | GGAUCAUCUCAAGUCUUACAUCACCCGUCU | 47 |
| | (FVII/mTTR) | AUUAUAGAGCAAGAACACUGCA | |
| | Strand3 (mTTR, | CAGUGUUCUUGCUCUAUAAUAGACG | 39 |
| | sense) | | |
| | Strand4 (HPRT, | GCACUUCAAAUCCAACAAAGUCUGGCA | 2 |
| | antisense) | | |
| Y-RNA (nick) | Strand1 | CCAGACUUUGUUGGAUUUGAAGUGCGGUGA | 69 |
| with P(-1)P | (HPRT/FVII) | UGUAAGACUUGAGAUGAUCCCA | |
| chemical | Strand2 | GGAUCAUCUCAAGUCUUACAUCACCCGUCU | 70 |
| modification | (FVII/mTTR) | AUUAUAGAGCAAGAACACUGCA | |
| | Strand3 | CAGUGUUCUUGCUCUAUAAUAGACG | 67 |
| | (mTTR, sense) | | |
| | Strand4 | GCACUUCAAAUCCAACAAAGUCUGGCA | 62 |
| | (HPRT, antisense) | | |

The four strands of Table 23 were each mixed in an amount of 2 nmoles and hybridized into double strands by incubation starting at 95° C. for 3 minutes, with a subsequent temperature decrease from 95° C. to 4° C. at a rate of −1.0° C./s in a thermal cycler (Bio-Rad T100™), to construct Y-RNA constructs with a nick. The Y-RNA constructs with a nick to includes arms that could be prepared into cleaved dsRNA targeting HRRT, FVII, and mouse TTR.

### Example 16-2

### Assay for In Vivo Gene Silencing Activity

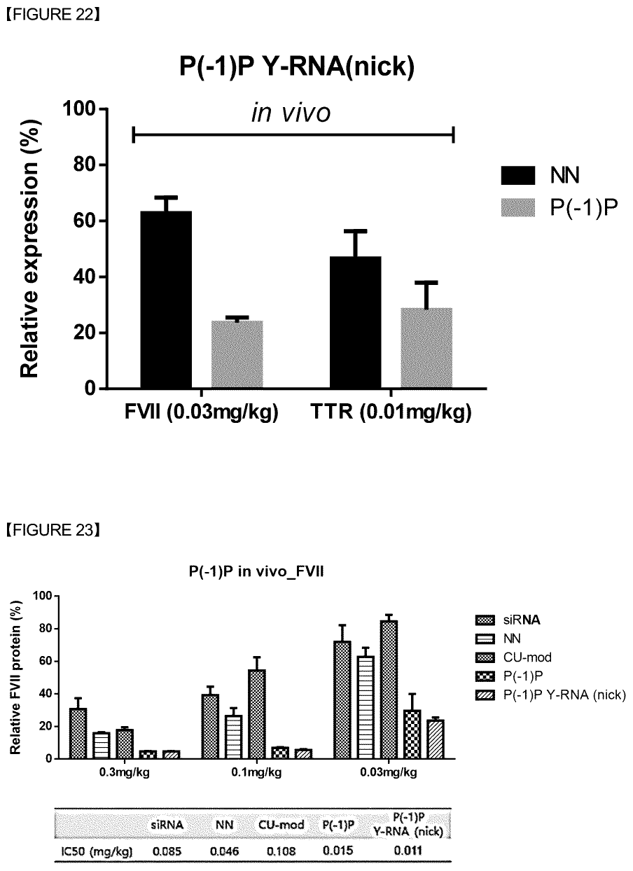

Dicer substrate Y-RNAs with a nick therein, which had a P(−1)P chemical modification introduced thereinto, were measured for in vivo gene silencing activity, and the results are depicted in FIG. 22.

Lipid nanopartides containing the Dicer substrate Y-RNA construct prepared in Example 16-1 were synthesized in a similar manner to that of Example 8-2 and administered by tail IV injection into C57bl/6NCrSlc mice (Charles River Labs, female, 18-22 g) with 6 io weeks of age. Expression levels of FVII and TTR were measured using the chromogenic assay and ELISA method as in Example 8-2, and the results are depicted in FIG. 22. The Dicer substrate RNAs targeting FVII and TTR were injected at doses of 0.03 mg/kg and 0.01 mg/kg to mice, respectively.

As shown in FIG. 22, both the Y-RNA constructs with a nick therein, which had a site-specific chemical modification made thereto, according to an embodiment exhibited an increased gene silencing effect in vivo, irrespective of the sequences (targets).

### Example 17

### In-Vito Gene Silencing Effect According to Concentration of Dicer Substrate RNA with P(−1)P Site-Specific Chemical Modification

In this Example, a Dicer substrate Y-RNA construct and a Y-RNA construct with a nick therein, both having P(−1)P site-specific chemical modifications made thereto, were prepared and examined for gene silencing effects in vivo according to concentrations thereof. The Y-RNA constructs are exemplified as shown in FIG. 9.

### Example 17-1

### Construction of Chemically Modified Dicer Substrate dsRNA

To construct Dicer substrate RNAs in various structures (linear dsRNA and Y-RNA with a nick), which could be prepared into deaved dsRNAs, strands having the sequences shown in Tables 24 and 25, below, with chemically modified nucleotides at the indicated positions, were synthesized in and purchased from BIONEER. In Tables 24 and 25, below, underlined nucleotides in bold had the sugar ring moiety in which the 2′-OH group was modified into a 2′-O-methyl group. The strands listed in Table 24 were designed to construct siRNAs and Dicer substrate RNAs, all targeting FVII while the strands listed in Table 25 were designed to construct siRNAs and Dicer substrate RNAs, all targeting TTR.

TABLE 24

| Strand | | Sequence (5′→3′) | SEQ ID NO: |
|---|---|---|---|
| Conventional | Sense strand | GGAUCAUCUCAAGUCUUACTT | 37 |
| siRNA (19 + 2) | Antisense strand | GUAAGACUUGAGAUGAUCCTT | 38 |
| Linear RNA | Sense strand | GGAUCAUCUCAAGUCUUACAUCACC | 41 |
| without chemical | Antisense strand | GGUGAUGUAAGACUUGAGAUGAUCCCA | 42 |
| modification | | | |
| Linear RNA with | Sense strand | GGAUCAUCUCAAGUCUUACAUCACC | 65 |
| with P(-1)P | Antisense strand | GGUGAUGUAAGACUUGAGAUGAUCCCA | 66 |
| chemical | | | |
| modification | | | |
| Linear RNA with | Sense strand | CACC | 55 |
| C/U sequence- | Antisense strand | GGUGAUGUAAGACUUGAGAUGAUCCCA | 56 |
| based | | | |
| modification | | | |
| Y-RNA (nick) with | Strand 1 | CCAGACUUUGUUGGAUUUGAAGUGCGG | 69 |
| P(-1)P chemical | (HPRT/FVII) | UGAUGUAAGACUUGAGAUGAUCCCA | |
| modification | Strand2 | GGAUCAUCUCAAGUCUUACAUCACCCG | 70 |
| | (FVII/mTTR) | UCUAUUAUAGAGCAAGAACACUGCA | |
| | Strand3 (mTTR, | CAGUGUUCUUGCUCUAUAAUAGACG | 67 |
| | sense) | | |
| | Strand4 | GCACUUCAAAUCCAACAAAGUCUGGCA | 62 |
| | (HPRT, antisense) | | |

TABLE 25

| Strand | | Sequence (5′→3′) | SEQ ID NO: |
|---|---|---|---|
| Conventional | Sense strand | CAGUGUUCUUGCUCUAUAATT | 35 |
| siRNA (19 + 2) | Antisense strand | UUAUAGAGCAAGAACACUGTT | 36 |
| Linear RNA | Sense strand | CAGUGUUCUUGCUCUAUAAUAGACG | 39 |
| without chemical | Antisense strand | CGUCUAUUAUAGAGCAAGAACACUGCA | 40 |
| modification | | | |
| Linear RNA with | Sense strand | CAGUGUUCUUGCUCUAUAAUAGACG | 67 |
| P(-1)P chemical | Antisense strand | CGUCUAUUAUAGAGCAAGAACACUGCA | 68 |
| modification | | | |
| Y-RNA (nick) with | Strand 1 | CCAGACUUUGUUGGAUUUGAAGUGCGG | 69 |
| P(-1)P chemical | (HPRT/FVII) | UGAUGUAAGACUUGAGAUGAUCCCA | |
| modification | Strand2 | GGAUCAUCUCAAGUCUUACAUCACCCG | 70 |
| | (FVII/mTTR) | UCUAUUAUAGAGCAAGAACACUGCA | |
| | Strand3 (mTTR, | CAGUGUUCUUGCUCUAUAAUAGACG | 67 |
| | sense) | | |
| | Strand4 | GCACUUCAAAUCCAACAAAGUCUGGCA | 62 |
| | (HPRT, antisense) | | |

The sense strands and antisense strands were each mixed in an amount of 10 nmoles and hybridized into double strands by incubation starting at 95° C. for 3 minutes, with a subsequent temperature decrease from 95° C. to 4° C. at a rate of −1.0° C./s in a thermal cycler (Bio-Rad T100™), to prepare siRNA and linear dsRNA.

The Y-RNA construct with a nick therein were prepared by mixing 5 nmoles of each of three or four strands and hybridizing the strands into double strands through incubation starting at 95° C. for 3 minutes, with a subsequent temperature decrease from 95° C. to 4° C. at a rate of −1.0° C./s in a thermal cycler (Bio-Rad T100™).

### Example 17-2

### Gene Silencing Effect According to Concentration

Lipid nanoparticles containing the various Dicer substrate Y-RNA constructs prepared in Example 17-1 were synthesized in a similar manner to that of Example 8-2 and administered ata dose of 100 μl containing various amounts of the RNA samples (0.03 mg/kg to 0.3 mg/kg) by tail IV injection into C57bl/6NCrSlc mice (Charles River Labs, female, 18-22 g) with 6 weeks of age. In-vivo gene silencing effects against the TTR target were examined in the siRNA as a control, the linear dsRNA without chemical modification, the linear dsRNA with a P(−1)P site-specific chemical modification, and the Y-RNA with a P(−1)P site-specific chemical modification and a nick.

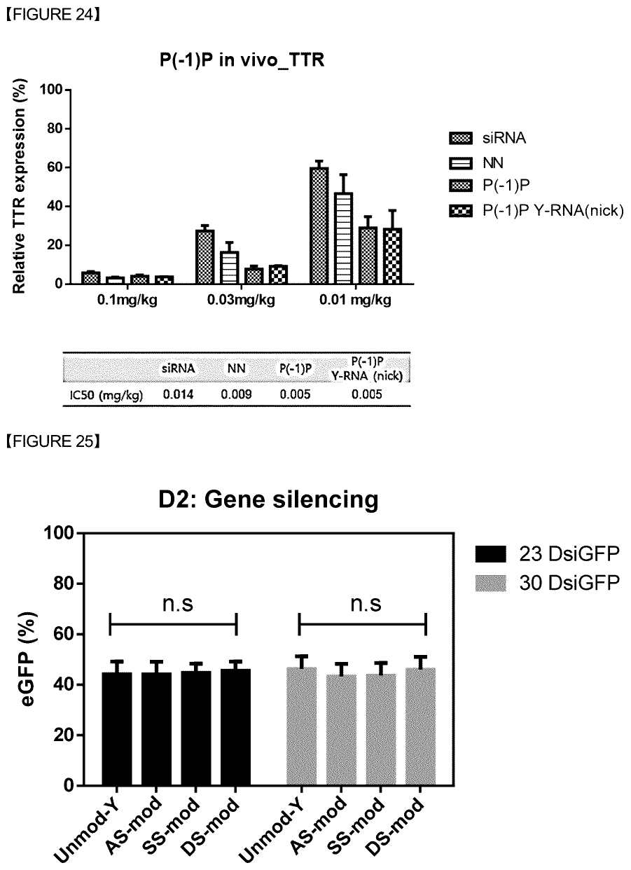

Expression levels of FVII and TTR were measured using the chromogenic assay and ELISA method as in Example 8-2, and the results are depicted in FIGS. 23 and 24, respectively. In addition, IC50 values accounting for inhibiting the expression of FVII and TTR in each control group and experimental group were calculated, and the results are tabulated in FIGS. 23 and 24, respectively.

As shown in FIGS. 23 and 24, the linear Dicer substrate dsRNA (P(−1)P), the Y-RNA construct (P(−1)P Y-RNA), and the Y-RNA construct with a nick (P(−1)P Y-RNA(nick)), each having a P(−1)P site-specific modification made thereto, were all observed to exhibit higher inhibitory effects against the expression of the target protein in vivo, with significantly lower IC50 values, than the control (NN), and these effects were consistent irrespective of the sequences (targets). Moreover, as shown in FIG. 23, the linear dsRNA and the Y-RNA construct with a nick, both having a P(−1)P site-specific chemical modification made thereto, exhibited remarkably higher inhibitory activity against the expression of FVII, with a significantly lower IC50 value, compared to those with C/U sequence-based chemical modifications.

### Example 18

### Effect According to Oength of Nucleic Acid Strand (In Vitro)

Dicer substrates to be prepared into dsRNAs targeting GFP were prepared. In this regard, the Dicer substrate dsRNAs were each designed in a site-specific modification manner (P(+)P site-specific chemical modification manner) to consist of a sense strand including chemically modified nudeotides at 1^{st}, 4^{th}, 5^{th}, 7^{th} and 14^{th} positions from the 5′ ends thereof and an antisense strand including chemically modified nucleotides at corresponding to positions to 2^{nd}, 3^{rd}, 6^{th}, 8^{th}, and 10^{th} to 13^{th} positions from the 5′ end of the sense strand complementary thereto. The Dicer substrate dsRNAs were as follows: (i) Dicer substrate dsRNA consisting of a 23 nt-long sense strand and a 25 nt-long antisense strand; and (ii) Dicer substrate dsRNA consisting of a 30 nt-long sense strand and a 32 nt-long antisense strand. Concrete modification positions in each group are given in Table 26, below. In Table 26, underlined nucleotides in bold had the sugar ring moiety in which the 2′-OH group was modified into a 2′-O-methyl group.

TABLE 26

| Strand | | Sequence (5′→3′) | SEQ ID NO: |
|---|---|---|---|
| Unmod-Y | sense strand | GCAAGCUGACCCUGAAGUUAUCA | 71 |
| (unmodified) | (23nt) | | |
| | antisense strand | UGAUAACUUCAGGGUCAGCUUGCCA | 72 |
| | (25nt) | | |
| AS-mod | sense strand | GCAAGCUGACCCUGAAGUUAUCA | 71 |
| (only antisense | (23nt) | | |
| strand modified) | antisense strand | UGAUAACUUCAGGGUCAGCUUGCCA | 74 |
| | (25nt) | | |
| SS-mod | sense strand | GCAAGCUGACCCUGAAGUUAUCA | 73 |
| (only sense | (23nt) | | |
| strand modified) | antisense strand | UGAUAACUUCAGGGUCAGCUUGCCA | 72 |
| | (25nt) | | |
| DS-mod | sense strand | GCAAGCUGACCCUGAAGUUAUCA | 73 |
| (both antisense | (23nt) | | |
| strand and | antisense strand | UGAUAACUUCAGGGUCAGCUUGCCA | 74 |
| sense strand | (25nt) | | |
| modified) | | | |
| Unmod-Y | sense strand | GCAAGCUGACCCUGAAGUUAUCACCUCACC | 75 |
| (unmodified) | (30nt) | | |
| | antisense strand | GGUGAGGUGAUAACUUCAGGGUCAGCUUGCCA | 76 |
| | (32nt) | | |
| AS-mod | sense strand | GCAAGCUGACCCUGAAGUUAUCACCUCACC | 75 |
| (only antisense | (30nt) | | |
| strand modified | antisense strand | GGUGAGGUGAUAACUUCAGGGUCAGCUUGCCA | 78 |
| | (32nt) | | |
| SS-mod | sense strand | GCAAGCUGACCCUGAAGUUAUCACCUCACC | 77 |
| (only sense | (30nt) | | |
| strand modified) | antisense strand | GGUGAGGUGAUAACUUCAGGGUCAGCUUGCCA | 76 |
| | (32nt) | | |
| DS-mod | sense strand | GCAAGCUGACCCUGAAGUUAUCACCUCACC | 77 |
| (both antisense | (30nt) | | |
| strand and | | | |
| sense strand | antisense strand | GGUGAGGUGAUAACUUCAGGGUCAGCUUGCCA | 78 |
| modified) | (32nt) | | |

The sense strands and antisense strands composed of the nucleotide sequences and having modified nucleotides (2-O-methyl group) as listed in Table 5 were synthesized in and purchased from BIONEER. The sense and antisense strands were each mixed at an amount of 100 pmoles and hybridized into double strands by incubation starting at 95° C. for 3 minutes, with a subsequent temperature decrease from 95° C. to 4° C. at a rate of −1.0° C./s in a thermal cycler (Bio-Rad T100™).

The eight Dicer substrate dsRNA samples thus prepared were applied to GFP-KB cells, and the expression level of the target gene GFP (eGFP) was measured in vitro in the same manner as in Example 1-3 through FACS. Thus, the results are depicted in FIG. 25.

As shown in FIG. 25, Dicer substrate dsRNAs with a P(+)P site-specific modification made to either or both of the antisense strand and sense strand (AS-mod, SS-mod, and DS-mod, respectively) did not decrease in gene silencing activity against GFP, compared to the chemically unmodified Dicer substrate RNA (Unmod-Y). In particular, this trend was observed consistently over the Dicer substrate dsRNAs including sense strands having a length of 25 nt (see FIG. 13), 23 nt (left panel of FIGS. 25), and 30 nt (right panel of FIG. 25). Therefore, even when the sense strand was 23 nt, 25 nt, or 30 nt long, the Dicer substrate dsRNAs with a P(+1)P site-specific modification are predicted to be superior in terms of in vivo serum stability and gene silencing activity to the Dicer substrate dsRNA with no modifications.

That is, the data imply that the position of the nucleotide modification plays an critical role in the increase in serum stability and/or gene silencing effect of Dicer substrate dsRNA is and the length of the gene sequence has no influences.

From the above description, those skilled in the art to which the present disclosure pertains will understand that the present disclosure may be embodied in other specific forms without changing the technical spirit or essential characteristics thereof. In this regard, it should be understood that the embodiments described above are illustrative in all respects and not restrictive. The scope of the present invention should be construed as being included in the scope of the present invention, rather than the above detailed description, all changes or modifications derived from the meaning and scope of the following claims and their equivalents.

1. A double-stranded nucleic acid molecule, comprising:
a sense strand 19 to 36 nt long; and
an antisense strand 21 to 38 nt long, including a sequence complementary to the sense strand, wherein the sense strand includes a chemically modified nucleotide at least one position selected from the 4^{th}, 5^{th}, 7^{th}, and 14^{th} from a 5′ end thereof, and wherein the antisense strand includes a chemically modified nucleotide at a position complementary to a nucleotide at least one position selected from the 2^{nd}, 3^{rd}, 6^{th}, 8^{th}, and 10^{th} to 13^{th} from the 5′ end of the sense strand.

2. The double-stranded nucleic acid molecule of claim 1, comprising:
a sense strand 19 to 36 nt long; and
an antisense strand 21 to 38 nt long, including a sequence complementary to the sense strand,
wherein the sense strand includes a chemically modified nucleotide at least one position selected from the 4^{th}, 5^{th}, 7^{th}, and 14^{th} from a 5′ end thereof,
wherein the antisense strand includes a chemically unmodified nucleotide at a position complementary to a nucleotide at the 7^{th} position from a 5′ end of the sense strand,
wherein the antisense strand includes,
(1) a chemically modified nucleotide at a position complementary to a nucleotide at the 8^{th} position from a 5′ end of the sense strand, or
(2) a chemically modified nucleotide at a position complementary to a nucleotide at the 8^{th} position from a 5′ end of the sense strand; and
a chemically modified nucleotide at a position complementary to a nucleotide present at least one position selected from the 2^{nd}, 3^{rd}, 6^{th}, 8^{th} and 10^{th} to 13^{th} from the 5′ end of a sense strand.

3. The double-stranded nucleic acid molecule of claim 1,
wherein the sense strand includes a chemically modified nucleotide at the 1^{st} position from a end thereof.

4. The double-stranded nucleic acid molecule of claim 1,
wherein the sense strand includes a chemically unmodified nucleotide at the 1^{st} position from a 5′ end thereof.

5. The double-stranded nucleic acid molecule of claim 1,
wherein the chemically modified nucleotide includes a sugar moiety modified with at least one selected from the group consisting of 2′-O-methyl, 2′-methoxyethoxy, 2′-fluoro, 2′-allyl, 2′-O-[2-(methylamino)-2-oxoethyl], 4′-thio, 4′-CH₂-O-2′-bridge, 4′-(CH₂)₂—O-2′-bridge, Z-LNA, 2′-amino, and 2′-O-(N-methykarbamate).

6. The double-stranded nucleic acid molecule of claim 1,
wherein the sense strand includes a chemically unmodified nucleotide at least one position selected from the group consisting of the 2^{nd}, 3^{rd}, 6^{th}, 8^{th} to 13^{th}, and 15^{th} to 36^{th} from a 5′ end thereof.

7. The double-stranded nucleic acid molecule of claim 1,
wherein the antisense strand includes a chemically unmodified at a position complementary to a nucleotide at least one position selected from the 1^{st}, 4^{th}, 5^{th}, 7^{th}, 9^{th}, and 14^{th} to 36^{th} from a 5′ end of the sense strand.

8. The double-stranded nucleic acid molecule of claim 2,
wherein the antisense strand includes a chemically unmodified nucleotide at a position complementary to a nucleotide at least one position selected from the 1^{st}, 4^{th}, 5^{th}, 9^{th}, and 14^{th} to 36^{th} from a 5′ end of the sense strand.

9. The double-stranded nucleic acid molecule of claim 1, capable of being endogenously cleaved by Dicer.

10. The double-stranded nucleic acid molecule of claim 1,
wherein the 3′ end of the sense strand and the 5′ end of the antisense strand are each a blunt end.

11. The double-stranded nucleic acid molecule of claim 1,
wherein the sense strand or the antisense strand has an overhang 1 to 5 nt long, on the 3′ end, the 5′ end or both of the 3′ and 5′ ends thereof.

12. The double-stranded nucleic acid molecule of claim 11,
wherein the overhang comprises a chemically unmodified nucleotide.

13. The double-stranded nucleic acid molecule of claim 1,
wherein the single-stranded nucleic acid molecule is in a hairpin structure.

14. The double-stranded nucleic acid molecule of claim 1,
wherein the antisense strand comprises a sequence complementary to an overall or partial sequence of a target gene.

15. The double-stranded nucleic acid molecule of claim 14, regulating an expression of the target gene.

16. The double-stranded nucleic acid molecule of claim 14,
wherein the target gene is selected from a protein-encoding gene, a proto-oncogene, an oncogene, a tumor suppressor gene, and a cell signaling gene.

17. The double-stranded nucleic acid molecule of claim 1, further comprising:
a polynucleotide 1 to 30 nt long at the 3′ end of the sense strand, and a polynucleotide 1 to 30 nt long at the 5′ end of the antisense strand.

18. The double-stranded nucleic acid molecule of claim 17,
wherein the polynucleotide extending from the 3′ end of the sense strand and the polynucleotide extending from the 5′ end of the antisense strand are not complementary to each other.

19. A radial nucleic acid molecule, comprising two to four entities of the double-stranded nucleic acid molecule of claim 1.

20. The radial nucleic acid molecule of claim 19, comprising two entities of the double-stranded nucleic acid molecule,
wherein the 3′ end of a sense strand in the first double-stranded nucleic acid molecule is linked to the 5′ end of an antisense strand in the second double-stranded nucleic acid molecule;
the 5′ end of an antisense strand in the first double-stranded nucleic acid molecule is linked to the 3′ end of a sense strand in the second double-stranded nucleic acid molecule; or
the combination thereof.

21. The radial nucleic acid molecule of claim 19, comprising three entities of the double-stranded nucleic acid molecule and exhibiting at least two of the following features (i) to
(i) linkage of the 3′ end of a sense strand in the first double-stranded nucleic acid molecule to the 5′ end of an antisense strand in the second double-stranded nucleic acid molecule;
(ii) linkage of the 5′ end of an antisense strand in the first double-stranded nucleic acid molecule to the 3′ end of a sense strand in the third double-stranded nucleic acid molecule; and
(iii) linkage of the 3′ end of a sense strand in the second double-stranded nucleic acid molecule to the 5′ end of an antisense strand in the third double-stranded nucleic acid molecule.

22. A method for inhibiting expression of a gene, comprising a step of administering a double-stranded nucleic acid molecule according to claim 1; a radial nucleic acid molecule comprising two to four entities of the double-stranded nucleic acid molecule; or a combination thereof,
to a subject in need of inhibiting expression of a gene.

23. The method of claim 22, wherein the gene is selected from a protein-encoding gene, a proto-oncogene, an oncogene, a tumor suppressor gene, and a cell signaling gene.

24. A method for preventing or treating a disease, comprising a step of administering a double-stranded nucleic acid molecule according to claim 1; a radial nucleic acid molecule including two to four entities of the double-stranded nucleic acid molecule; or a combination thereof,
to a subject in need of preventing or treating a disease,
wherein the disease is at least one selected from the group consisting of cancer, proliferative disease, digestive disease, kidney disease, neurological disease, mental disease, blood and tumor disease, cardiovascular disease, respiratory disease, endocrine disease, infectious disease, musculoskeletal disease, gynecological disease, genitourinary disease, skin disease, and ophthalmic disease.

25-29. (canceled)
